(19) United States
(12) Patent Application Publication
(10) Pub. No.: US 2024/0368209 A1
(43) Pub. Date: Nov. 7, 2024

(54) GEMCITABINE ANTICANCER DERIVATIVE AND ANTICANCER PHARMACEUTICAL USE

(71) Applicant: ASCENTAWITS PHARMACEUTICALS, LTD., Pingshan District, Shenzhen, Guangdong (CN)

(72) Inventors: Anrong LI, Pingshan District, Shenzhen, Guangdong (CN); Tianyang QI, Pingshan District, Shenzhen, Guangdong (CN); Jianxin DUAN, Pingshan District, Shenzhen, Guangdong (CN); Fanying MENG, San Francisco, CA (US); Teng MENG, Pingshan District, Shenzhen, Guangdong (CN); Mengyun ZHANG, Pingshan District, Shenzhen, Guangdong (CN)

(21) Appl. No.: 18/568,641

(22) PCT Filed: Jun. 10, 2022
(86) PCT No.: PCT/CN2022/098082

§ 371 Date: Dec. 8, 2023

(30) Foreign Application Priority Data
Jun. 11, 2021 (CN) 202110653130.5

(51) Int. Cl. C07H 19/073; A61K 31/7068; A61P 35/00; C07H 19/24

(52) U.S. Cl. CPC C07H 19/073; A61K 31/7068; A61P 35/00; C07H 19/24

(57) ABSTRACT

The present invention provides gemcitabine anticancer derivatives of formula (III), which have the potential to be developed into AKR1C3-activated anticancer drugs, and the anticancer pharmaceutical use thereof,.

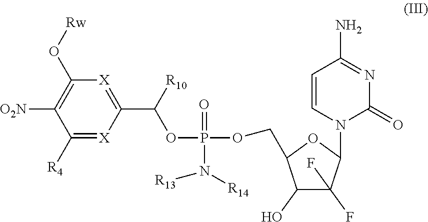

### CROSS-REFERENCE TO RELATED APPLICATIONS

This application claims priority to co-pending PCT International Application Serial No. PCT/CN2022/098082 filed Jun. 10, 2022, which claims priority to CN Patent Application No. 202110653130.5 filed on Jun. 11, 2021, the entire content of both of which is incorporated herein by reference.

### TECHNICAL FIELD

The present invention, belonging to the field of research and development of anticancer drugs, relates to a series of AKR1C3 enzyme-activated gemcitabine anticancer derivatives obtained by further research and development of the compound TH3057 disclosed in Patent Application No. PCT/US2016/025665 entitled NITROBENZYL DERIVATIVES OF ANTI-CANCER AGENTS (Publication No. WO2016/161342A1), which corresponds to Chinese Patent Application No. 201680020013.2 (Publication No. CN108136214A).

### BACKGROUND ART

The invention by Jian-xin Duan et al., entitled NITROBENZYL DERIVATIVES OF ANTI-CANCER AGENTS with Patent Application No. PCT/US2016/025665 and Publication No. WO2016/161342A1, corresponding to Chinese Patent Application No. 201680020013.2 with Publication No. CN108136214A, discloses a series of nitrobenzyl derivatives:

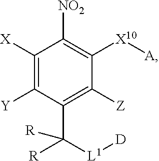

which actually is an AKR1C3 activated anticancer drug obtained by bonding a nitrobenzyl structure to an anticancer drug residue by L¹, wherein the linker L¹ is defined to have different structures for different anticancer drug residues D.

In particular, in this application, the inventors designed a derivative of gemcitabine combined with a nitrobenzyl structure, which has the following structure:

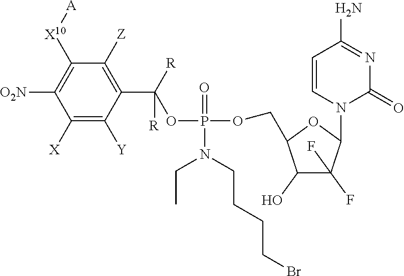

and the three specific compounds:

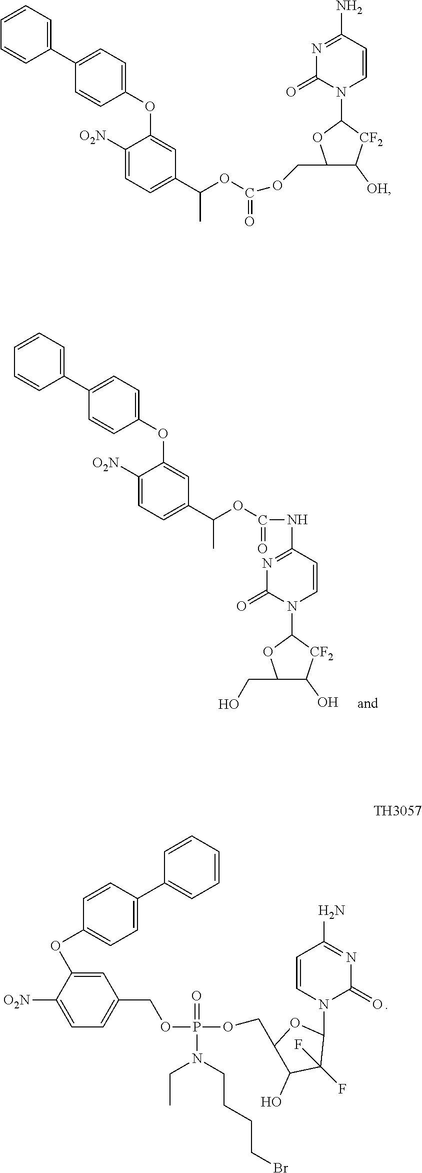

The compound TH3057 was further synthesized, and tested for its cell proliferation inhibition activity (IC₅₀ values) against the H460 cell line with high expression of AKR1C3 enzyme, without and with the addition of AKR1C3 enzyme inhibitor, with the IC₅₀ values being 0.2 μmol/L and 5 μmol/L respectively.

### SUMMARY OF THE INVENTION

The inventors continued further research and found that not all the derivatives designed in the above invention that combine gemcitabine with a nitrobenzyl structure were AKR1C3 activated anticancer drugs, or compounds similar to TH3057. Not all the compounds obtained from the gemcitabine residue bonded to a nitrobenzyl structure by the linker L¹ were AKR1C3 activated anticancer drugs. They were tested to demonstrate that not all of them could be activated by AKR1C3 enzyme or to have extremely low activation ratios. Therefore, based on the compounds designed in the aforementioned invention, the inventors designed and synthesized a series of gemcitabine anticancer derivatives, which were confirmed to have the ability to be activated by AKR1C3 enzyme or have a higher activation ratio. And these compounds may have better potential to be developed as AKR1C3 activated anticancer drugs than TH3057. Therefore, the present invention provides a new possibility or idea for designing and developing AKR1C3 activated anticancer drugs with higher specific activation ratios.

The present invention provides gemcitabine anticancer derivatives with new structures, which are AKR1C3 activated anticancer drugs.

A gemcitabine derivative of formula (III), or a pharmaceutically acceptable salt, a solvate, an isotopic variant, or an isomer thereof:

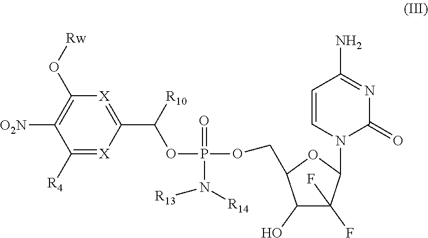

wherein,
two X's are each independently CR¹⁵ or N;
R₁₃ and R₁₄ are each independently hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl, and R₁₃ and R₁₄ are not hydrogen at the same time;
R₁₀ is hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
or R₁₀ and R₁₃ or R₁₄ may be connected to form a 5-9-membered ring under the condition that they meet the above definitions for R₁₀, R₁₃ and R₁₄;
R₄ and R¹⁵ are each independently hydrogen; halogen; C₁-C₆ alkyl; cycloalkyl; alkenyl; alkynyl; alkoxy; cyano; 5-20-membered heterocyclyl; C₆-C₂₀ aryl; or halogen-substituted C₁-C₆ alkyl, cycloalkyl, alkenyl, alkynyl, alkoxyl, 5-20-membered heterocyclyl or C₆-C₂₀ aryl;
or R₁₀ and R¹⁵ may form a 4-12-membered cyclic hydrocarbon or heterocycle under the condition that they meet the above definitions for R₁₀ and R¹⁵;
R_{W} is

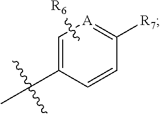

A is CR¹⁶ or N, and the position of A may be changed on the ring;
R¹⁶ is hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
R₆ and R₇ meet the following conditions:
R₆ and R₇ are each independently hydrogen; halogen; cyano; hydroxyl; C₁-C₆ alkyl, cycloalkyl, alkenyl, alkynyl, alkoxyl, 5-20-membered heterocyclyl or C₆-C₂₀ aryl; or halogen-substituted C₁-C₆ alkyl, cycloalkyl, alkenyl, alkynyl, alkoxyl, 5-20-membered heterocyclyl or C₆-C₂₀ aryl; or cyano-substituted C₁-C₆ alkyl, cycloalkyl, alkenyl, alkynyl, alkoxyl, 5-20-membered heterocyclyl or C₆-C₂₀ aryl; or hydroxyl-substituted C₁-C₆ alkyl, cycloalkyl, alkoxyl, 5-20-membered heterocyclyl or C₆-C₂₀ aryl; or —CONR¹¹R¹²; or —CH₂NR¹¹R¹²;
or R₆ and R₇ are connected to form
a 5-8-membered single or fused heterocycle containing at least one N or S or O, or two or three of N, S and O at the same time;
or a 5-8-membered single or fused heterocycle containing at least one N or S or O, or two or three of N, S and O at the same time, which is C₁-C₆ alkyl-substituted;
R₆ and CR¹⁶ may be connected to form a 5-9-membered ring, heterocycle, or aromatic heterocycle;
R¹¹ and R¹² meet the following conditions:
R¹¹ and R¹² are each independently C₁-C₆ alkyl, or halogen-substituted C₁-C₆ alkyl, or R¹¹ and R¹² form a 5-7-membered ring with N from —CONR¹¹R¹², or form a 5-7-membered ring with N from —CH₂NR¹¹R¹² under the condition that they meet the above definitions for R¹¹ and R¹².

Preferably, the compound with the structural formula of formula (III) includes compounds of formula (I) and formula (II) below:

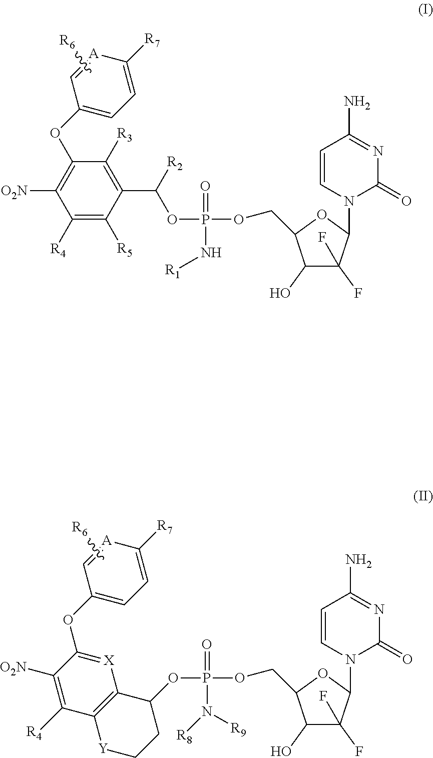

wherein,
Y is CR^{a}R^{b}, NR^{a}, or O, and the position of Y may be changed between the 4 positions excluding 2 carbon atoms shared with the benzene ring;
R^{a} and R^{b} meet the following conditions:
R^{a} and R^{b} are each independently hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
R₁, R₂, R₈ and R₉ meet the following conditions:
R₁ is C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
R₂ is hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
or R₁ and R₂ may be connected to form a 5-9-membered ring under the condition that they meet the above definitions for R₁ and R₂;
R₈ and R₉ are each independently hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl, and R₈ and R₉ are not hydrogen at the same time;
or R₈ and R₉ may be connected to form a 5-9-membered ring under the condition that they meet the above definitions for R₈ and R₉;
R₃, R₄ and R₅ meet the following conditions:
R₃, R₄ and R₅ are each independently hydrogen; halogen; C₁-C₆ alkyl; cycloalkyl; alkenyl; alkynyl; alkoxy; cyano; 5-20-membered heterocyclyl; C₆-C₂₀ aryl; or halogen-substituted C₁-C₆ alkyl, cycloalkyl, alkenyl, alkynyl, alkoxyl, 5-20-membered heterocyclyl or C₆-C₂₀ aryl.

R₆ above is connected to the benzene or pyridine ring via a wavy line, and the position of connection is variable and may be one of positions 2/3/5/6 for the specific compound of this structure.

The substitution has a broad meaning. It can be mono-substitution (only one H on a C atom in a benzene ring and the like can be substituted), or multi-substitution, which include multiple substitutions on a certain C atom, i.e., di-substitution and tri-substitution (such as gem-difluoromethyl and gem-trifluoromethyl) or separate substitution on different C atoms in a ring (such as perfluorobenzene).

Alkyl refers to monovalent saturated aliphatic hydrocarbyl groups having more than 1 carbon atom and, in some embodiments, from 1 to 6 carbon atoms. “C_{x-y} alkyl” refers to alkyl groups having from x to y carbon atoms. This term includes, by way of example, linear and branched hydrocarbyl groups (chain alkyl) such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), s-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—), etc.

When alkyl and cycloalkyl groups are recited as alternatives, alkyl specifically refers to chain alkyl and is distinguished from cycloalkyl; when alkyl appears alone, its meaning is broad, including chain alkyl and cycloalkyl groups, such as C₁-C₆ alkyl and halogen-substituted C₁-C₆ alkyl including C₁-C₆ chain alkyl and cyclic alkyl, and halogen-substituted C₁-C₆ chain alkyl and cyclic alkyl, respectively.

Cycloalkyl or cyclic alkyl refers to a saturated or partially saturated cyclic group of more than 3 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g., 5,6,7,8,-tetrahydronaphthalene-5-yl). Examples of cycloalkyl groups include, for instance, adamantyl, cyclopropyl, cyclobutyl, cyclopentyl, cyclooctyl, and cyclohexenyl. “Cycloalkylene” refers to a divalent cycloalkyl radical having the appropriate hydrogen content.

Alkenyl refers to a linear or branched hydrocarbyl or cyclic hydrocarbyl group having more than 2 carbon atoms and in some embodiments from 1 to 6 carbon atoms and having at least 1 site of vinyl unsaturation (>C═C<). For example, C_{x-y} alkenyl refers to alkenyl groups having from x to y carbon atoms and is meant to include, for example, ethenyl, propenyl, 1,3-butadienyl, and the like.

Alkynyl refers to a linear monovalent hydrocarbyl radical or a branched monovalent hydrocarbyl radical or cyclic hydrocarbyl radical having more than 2 carbon atoms and in some embodiments from 2 to 6 carbon atoms or 2 to 4 carbon atoms and containing at least one triple bond. For example, C₂-C₆ alkynyl includes ethynyl, propynyl, and the like.

“C₆-C₂₀ aryl” refers to an aromatic group of from 6 to 20 carbon atoms and no ring heteroatoms and having a single ring (e.g., phenyl) or multiple condensed (fused) rings (e.g., naphthyl or anthryl), or hydrocarbyl groups (alkyl, cycloalkyl, alkenyl, alkynyl) are attached to the ring so that the total number of carbon atoms including the ring and the hydrocarbyl groups on the ring is 6-20. For multiple ring systems, including fused, bridged, and spiro ring systems having aromatic and non-aromatic rings that have no ring heteroatoms, the term “aryl” or “Ar” applies when the point of attachment is at an aromatic carbon atom (e.g., 5,6,7,8-tetrahydronaphthalene-2-yl is an aryl group as its point of attachment is at the 2− position of the aromatic phenyl ring).

“Halogen” refers to one or more of fluorine, chlorine, bromine, and iodine.

“Cyano” refers to a —C≡N group.

“Heteroaryl” refers to an aromatic group of 1 to 6 heteroatoms selected from the group consisting of oxygen, nitrogen, and sulfur and includes single ring (e.g., imidazol-2-yl and imidazol-5-yl) and multiple ring systems (e.g., imidazopyridyl, benzotriazolyl, benzimidazol-2-yl and benzimidazol-6-yl). For multiple ring systems, including fused, bridged, and spiro ring systems having aromatic and non-aromatic rings, the term “heteroaryl” applies if there is at least one ring heteroatom, and the point of attachment is at an atom of an aromatic ring (e.g., 1,2,3,4-tetrahydroquinolin-6-yl and 5,6,7,8-tetrahydroquinolin-3-yl). In some embodiments, the nitrogen and/or the sulfur ring atom(s) of the heteroaryl group are optionally oxidized to provide for the N-oxide (N→O), sulfinyl, or sulfonyl moieties. The term heteroaryl includes, but is not limited to, acridinyl, azocinyl, benzimidazolyl, benzofuranyl, benzothiofuranyl, benzothiophenyl, benzoxazolyl, benzothiazolyl, benzotriazolyl, benzotetrazolyl, benzisoxazolyl, benzisothiazolyl, benzothienyl, benzimidazolinyl, carbazolyl, NH-carbazolyl, carbolinyl, chromanyl, chromenyl, cinnolinyl, dithiazinyl, furanyl, furazanyl, imidazolidinyl, imidazolinyl, imidazopyridyl, imidazolyl, indazolyl, indolenyl, indolinyl, indolizinyl, indolyl, isobenzofuranyl, isochromanyl, isoindazolyl, isoindolinyl, isoindolyl, isoquinolinyl, isoquinolyl, isothiazolyl, isoxazolyl, naphthyridinyl, octahydroisoquinolinyl, oxadiazolyl, oxazolidinyl, oxazolyl, pyrimidinyl, phenanthridinyl, phenanthrolinyl, phenazinyl, phenothiazinyl, phenoxathiinyl, phenoxazinyl, phthalazinyl, piperazinyl, pteridinyl, purinyl, pyranyl, pyrazinyl, pyrazolidinyl, pyrazolinyl, pyrazolyl, pyridazinyl, pyridooxazolyl, pyridoimidazolyl, pyridothiazolyl, pyridinyl, pyridyl, pyrimidinyl, pyrrolyl, quinazolinyl, quinolinyl, quinoxalinyl, quinuclidinyl, tetrahydroisoquinolinyl, tetrahydroquinolinyl, tetrazolyl, thiadiazinyl, thiadiazolyl, thianthrenyl, thiazolyl, thienyl, thienothiazolyl, thienooxazolyl, thienoimidazolyl, thiophenyl, triazinyl and xanthenyl.

“Heterocyclic” or “heterocycle” or “heterocycloalkyl” or “heterocyclyl” has the same meaning, refers to a saturated or partially saturated cyclic group having from 1 to 14 carbon atoms and from 1 to 6 heteroatoms selected from the group consisting of nitrogen, sulfur, or oxygen and includes single ring and multiple ring systems which include fused, bridged, and spiro ring systems. For multiple ring systems having aromatic and/or non-aromatic rings, the term “heterocyclic”, “heterocycle”, “heterocycloalkyl”, or “heterocyclyl” applies when there is at least one ring heteroatom, and the point of attachment is at an atom of a non-aromatic ring (e.g., 1,2,3,4-tetrahydroquinoline-3-yl, 5,6,7,8-tetrahydroquinoline-6-yl, and decahydroquinolin-6-yl). In some examples, the heterocyclic groups herein are 3-15-membered, 4-14-membered, 5-13-membered, 7-12-membered, or 5-7-membered heterocycles. In some other examples, the heterocycles contain 4 heteroatoms. In some other examples, the heterocycles contain 3 heteroatoms. In another example, the heterocycles contain up to 2 heteroatoms. In some examples, the nitrogen and/or sulfur atom(s) of the heterocyclic group are optionally oxidized to provide for the N-oxide, sulfinyl, or sulfonyl moieties. Heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

In this application, heterocycles or heterocyclyl groups include the aforementioned “heteroaryl” and “heterocycle”, but do not include “aryl”. A 5-8-membered heterocycle is a ring composed of 5-8 atoms, including heteroatoms (oxygen, nitrogen, and sulfur) and carbon atoms.

Preferably, R₁, R₈ and R₉ are each independently C₁-C₅ alkyl, halogen-substituted C₁-C₅ alkyl, C₃-C₆ cycloalkyl or C₆-C₂₀ aryl.

More preferably, R₁, R₈ and R₉ are each independently methyl, ethyl, n-propyl, isopropyl, isobutyl, t-butyl, neopentyl, bromoethyl, 1,1,1-trifluoroethyl, C₃-C₆ cycloalkyl or benzyl.

Preferably, R₂ is hydrogen, methyl, or trifluoromethyl.

Preferably, R₁ and R₂ in formula (I) are connected to form a 6-membered ring.

Preferably, R₃, R₄ and R₅ are each independently hydrogen.

Preferably, R₆ is hydrogen, fluorine, fluorophenyl, or

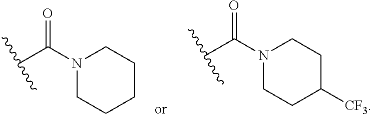 [SMILES: *C(=O)N1CCCCC1 ; "or" ; *C(=O)N1CCC(C(F)(F)F)CC1]

Preferably, R₇ is hydrogen, —CF₃, phenyl, chlorophenyl, fluorophenyl, —CF₃-substituted phenyl, fluoropyridyl, or

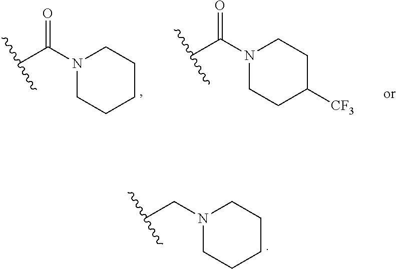 [SMILES: *C(=O)N1CCCCC1 ; "," ; *C(=O)N1CCC(C(F)(F)F)CC1 ; "or" ; *CN1CCCCC1 ; "."]

In chlorophenyl, fluorophenyl, —CF₃-substituted phenyl, and fluoropyridyl mentioned above, chlorine, fluorine, and —CF₃ may substitute at any substitutable position of the corresponding benzene ring and pyridine, and the number of substitutions may be more than one. For example, for chlorophenyl, it includes o-chlorophenyl, m-chlorophenyl, p-chlorophenyl, 2,3-dichlorophenyl, 2,4-dichlorophenyl, 2,5-dichlorophenyl, 2,6-dichlorophenyl, 2,3,4-trichlorophenyl 2,3,5-trichlorophenyl, 2,3,6-trichlorophenyl, 3,4,5-trichlorophenyl, 3,4,6-trichlorophenyl, 4,5,6-trichlorophenyl, and corresponding tetrachlorophenyl and pentachlorophenyl.

Preferably, R₆ and R₇ are connected to form a 5-8-membered heterocycle, C₁-C₆ alkyl-substituted 5-8-membered heterocycle containing N or S or O, or any two or three of N, S and O at the same time; and the 5-8-membered heterocycle shares 2 atoms with the connected benzene ring or pyridine ring to form a fused ring.

Further preferably, R₆ and R₇ are connected to form a 5-8-membered heterocycle containing 2 N atoms, or C₁-C₆ alkyl-substituted 5-8-membered heterocycle; more preferably, R₆ and R₇ are connected to form a pyrimidine, or C₁-C₆ alkyl-substituted pyrimidine; or R₆ and R₇ are connected to form a 5-8-membered heterocycle containing 1 N atom, or C₁-C₆ alkyl-substituted 5-8-membered heterocycle, and more preferably, R₆ and R₇ are connected to form a pyridine, or C₁-C₆ alkyl-substituted pyridine.

Preferably, the compound with the structural formula of formula (I) includes compounds with the structures of formulas I-1 to I-5 below:

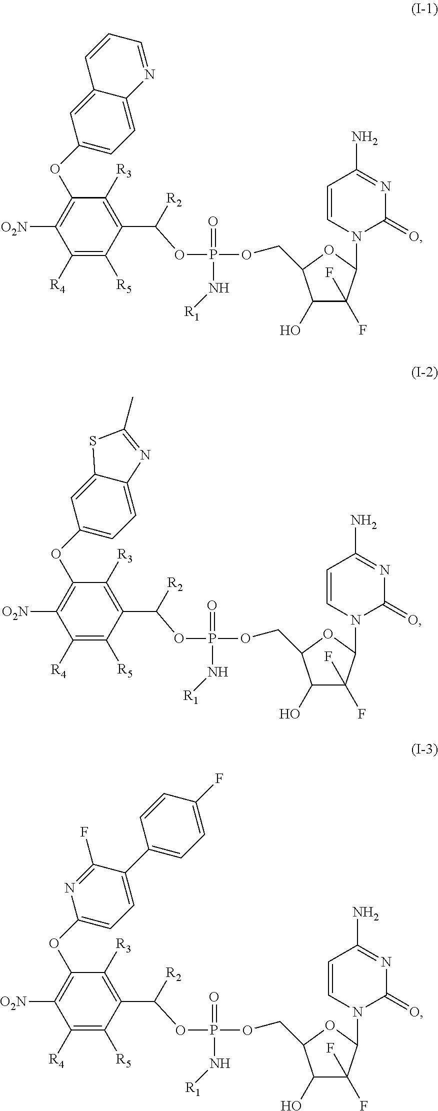

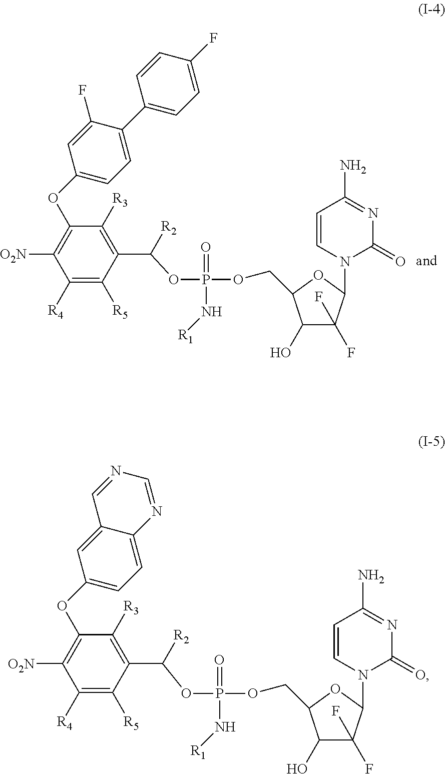

wherein definitions of the substituents are as above.

Preferably, the compound with the structural formula of formula (II) includes compounds with the structures of formulas II-1 to II-6 below:

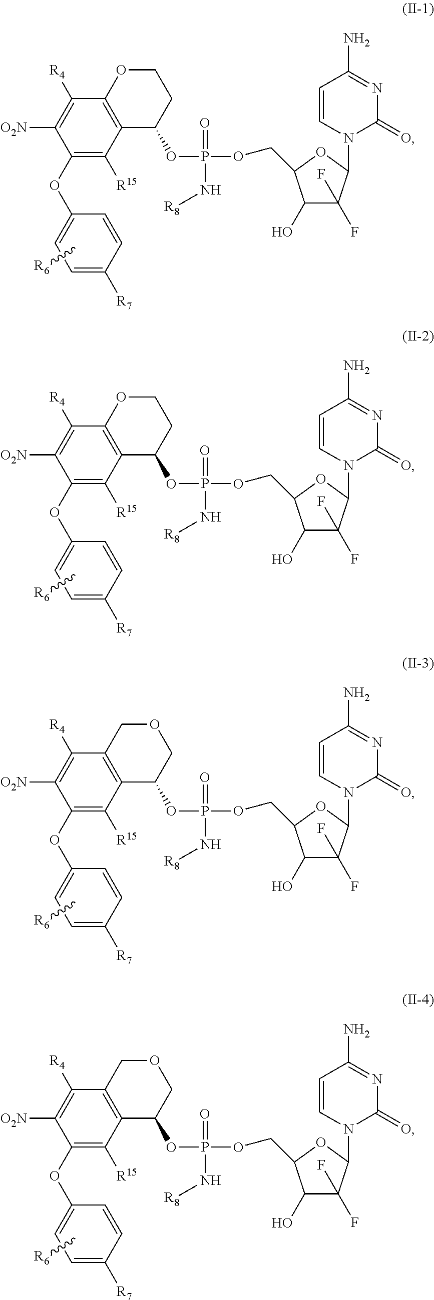

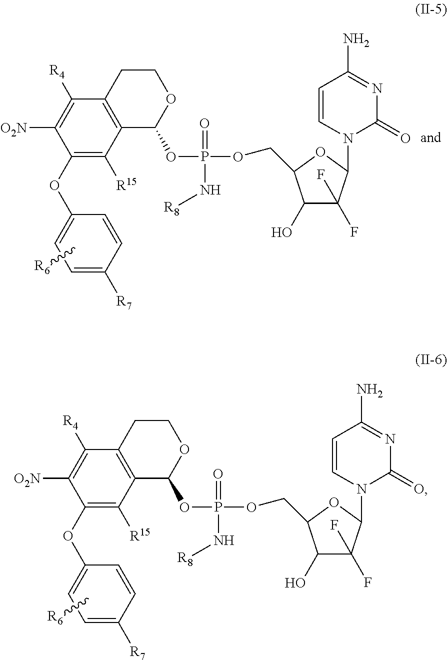

wherein definitions of the substituents are as above.

In this application, the structure of formula (I) substituted by isotope deuterium includes a compound with the structure of formula I-6 below:

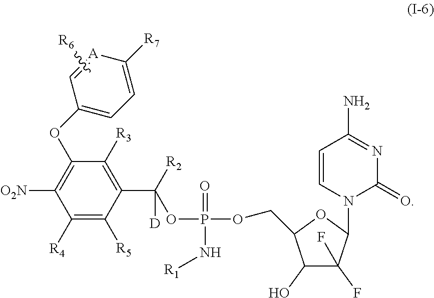

Preferably, the structure of formula (I) substituted by isotope deuterium includes compounds with the structures of formulas I-7 and I-8 below:

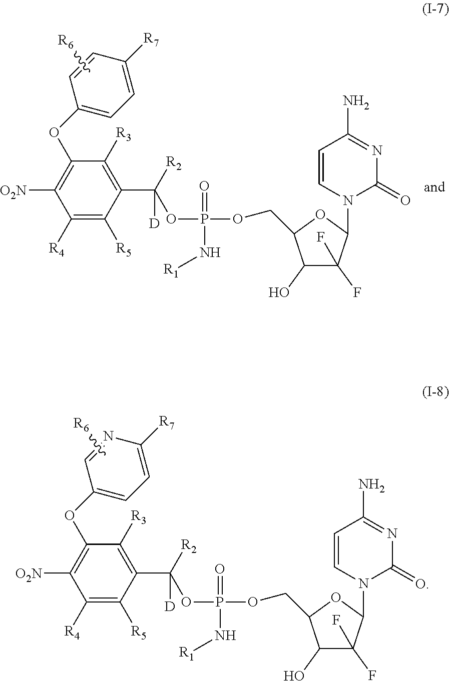

Further preferably, the structure of formula (I) substituted by the isotope deuterium includes compounds with the structures of formulas I-9 to I-13 below:

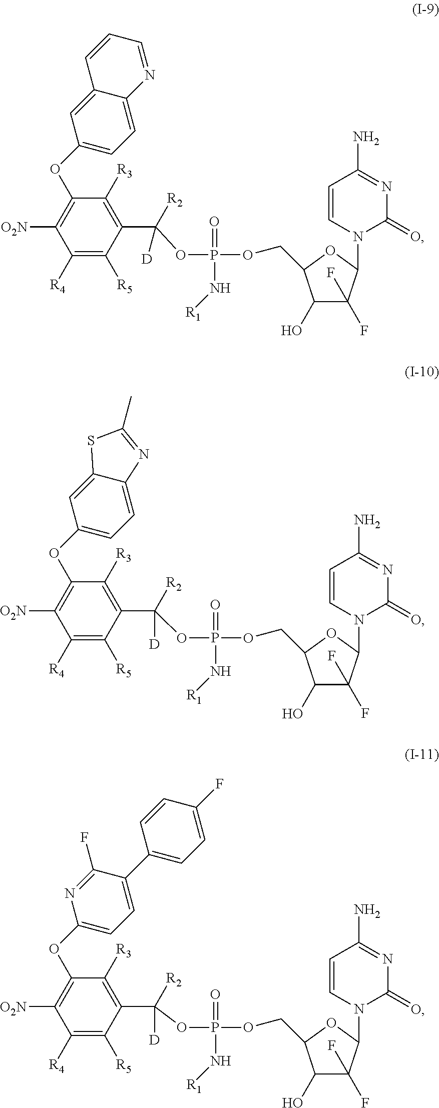

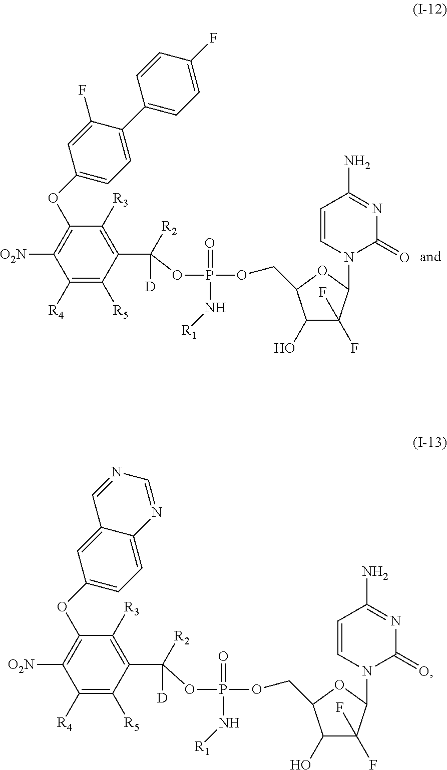

wherein definitions of the substituents are as above.

In this application, the structure of formula (II) substituted by isotope deuterium includes compounds with the structures of formulas II-7 to II-12 below:

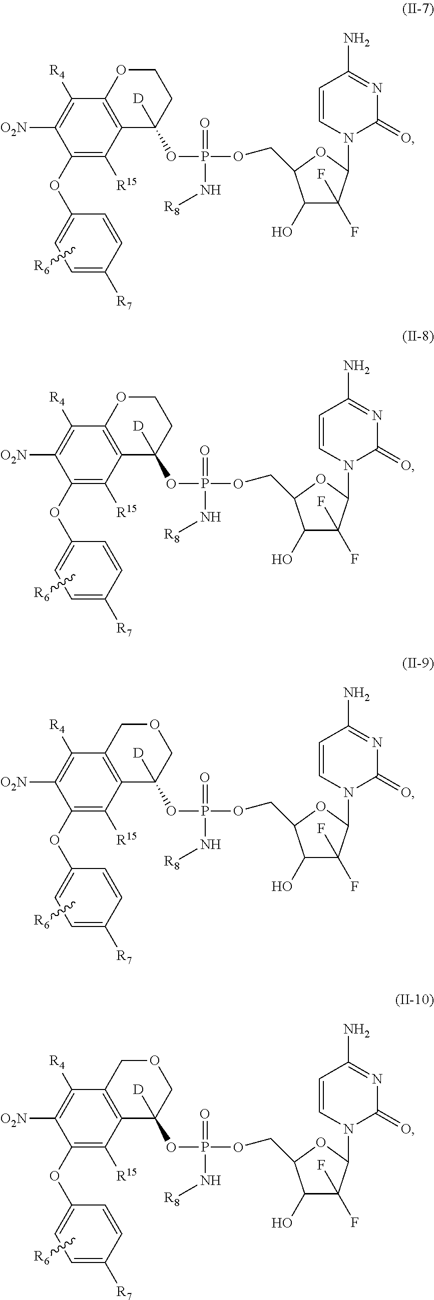

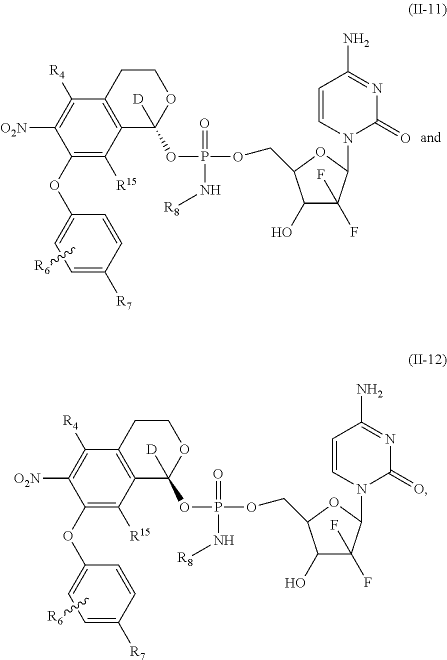

wherein, definitions of the substituents are as above.

Preferably, hydrogen atom(s) in R₁ are substituted by at least one deuterium.

Preferably, formula (I) is selected from compounds of the following structures:

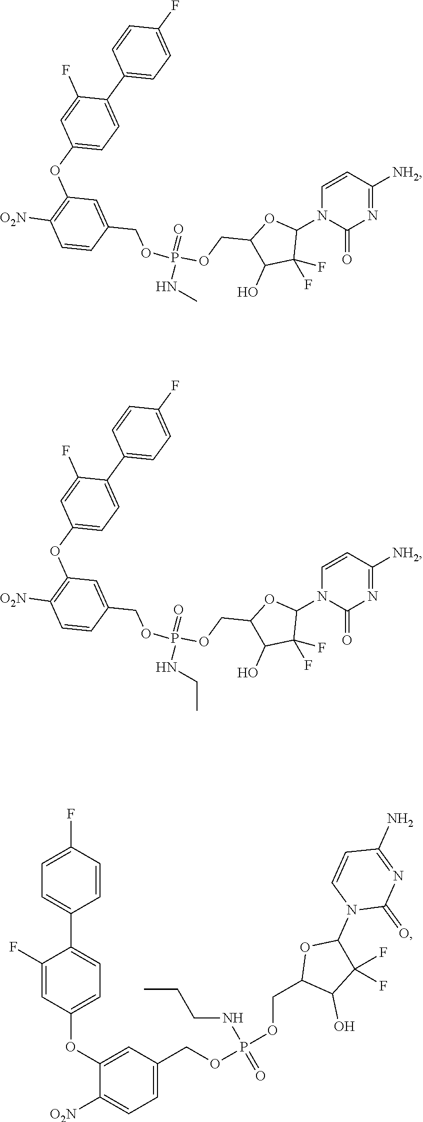 [SMILES: CNP(=O)(OCc1ccc([N+](=O)[O-])c(Oc2ccc(-c3ccc(F)cc3)c(F)c2)c1)OCC1OC(n2ccc(N)nc2=O)C(F)(F)C1O ; CCNP(=O)(OCc1ccc([N+](=O)[O-])c(Oc2ccc(-c3ccc(F)cc3)c(F)c2)c1)OCC1OC(n2ccc(N)nc2=O)C(F)(F)C1O ; CCCNP(=O)(OCc1ccc([N+](=O)[O-])c(Oc2ccc(-c3ccc(F)cc3)c(F)c2)c1)OCC1OC(n2ccc(N)nc2=O)C(F)(F)C1O]

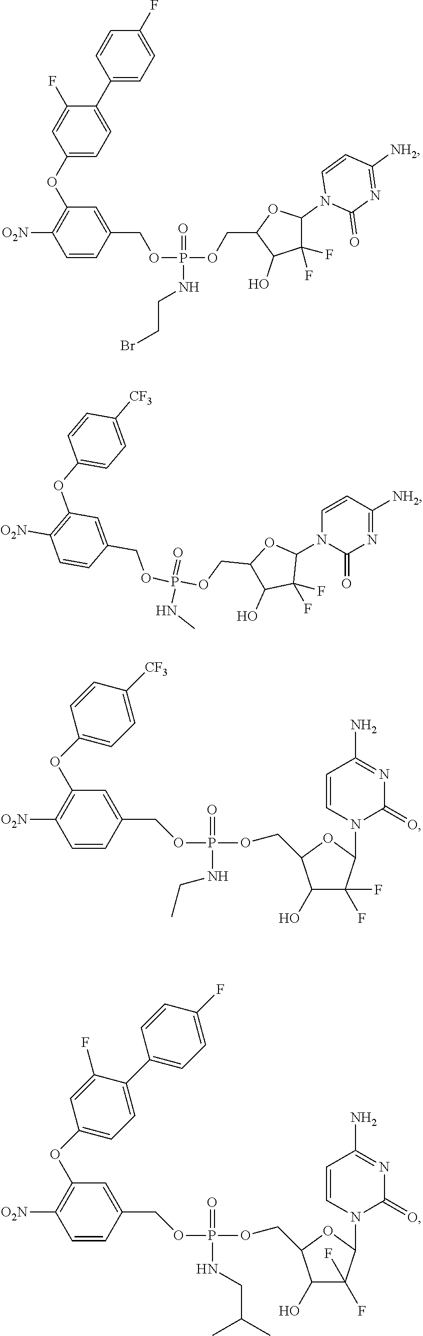

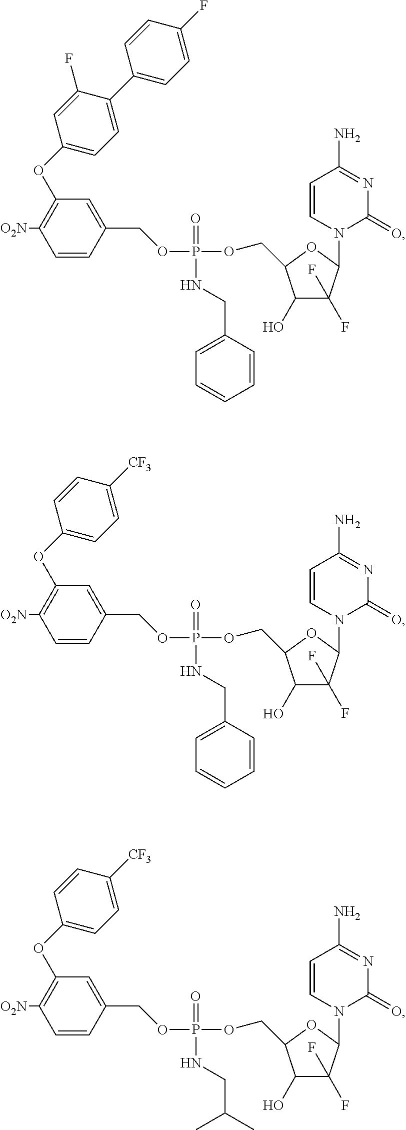

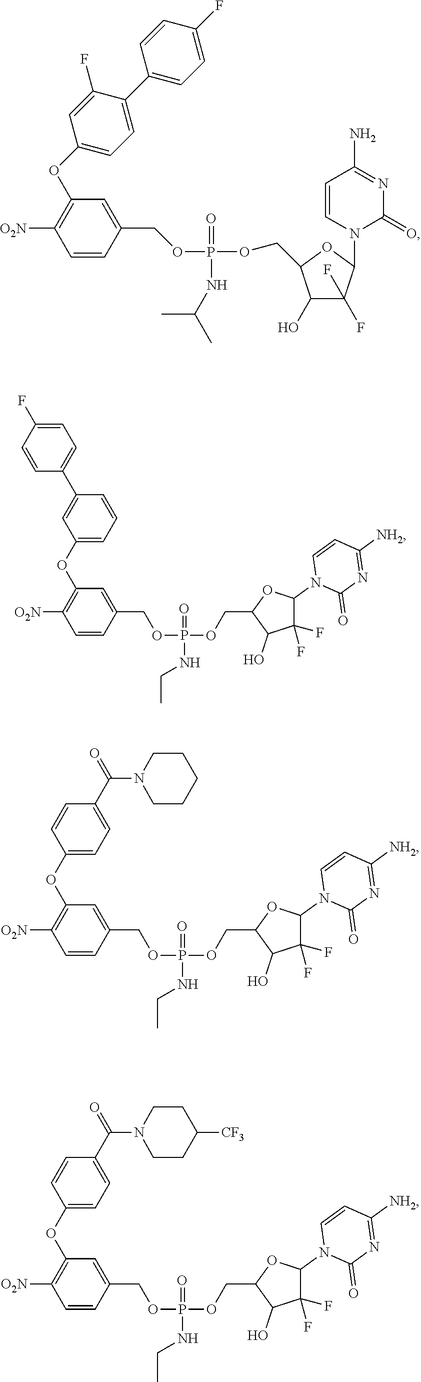

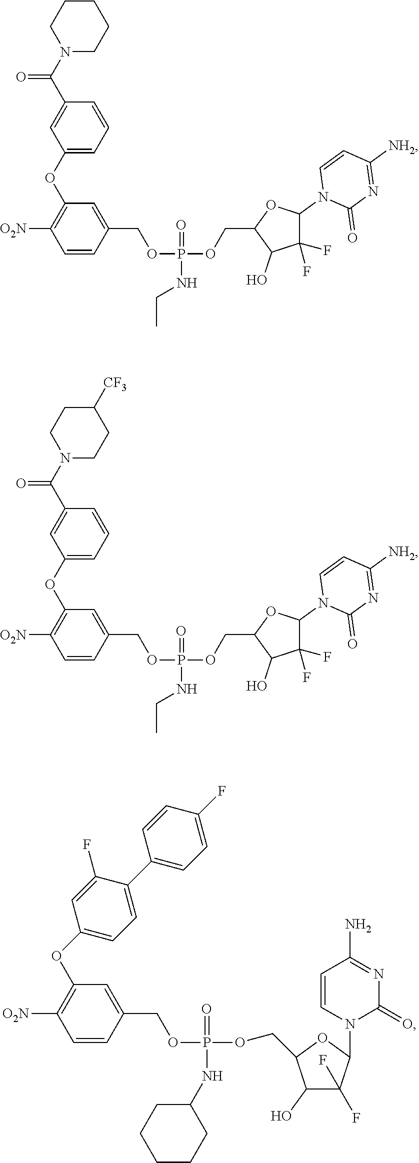

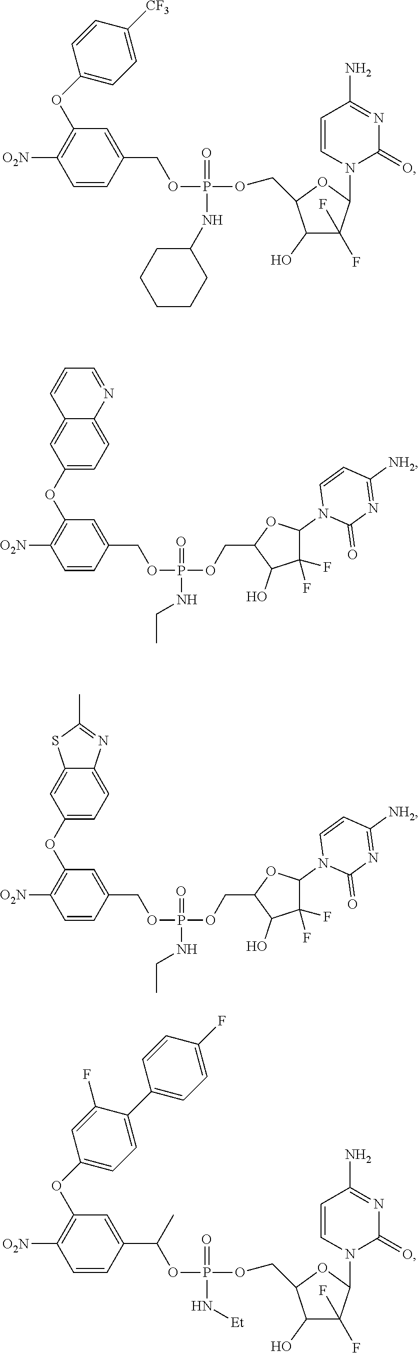

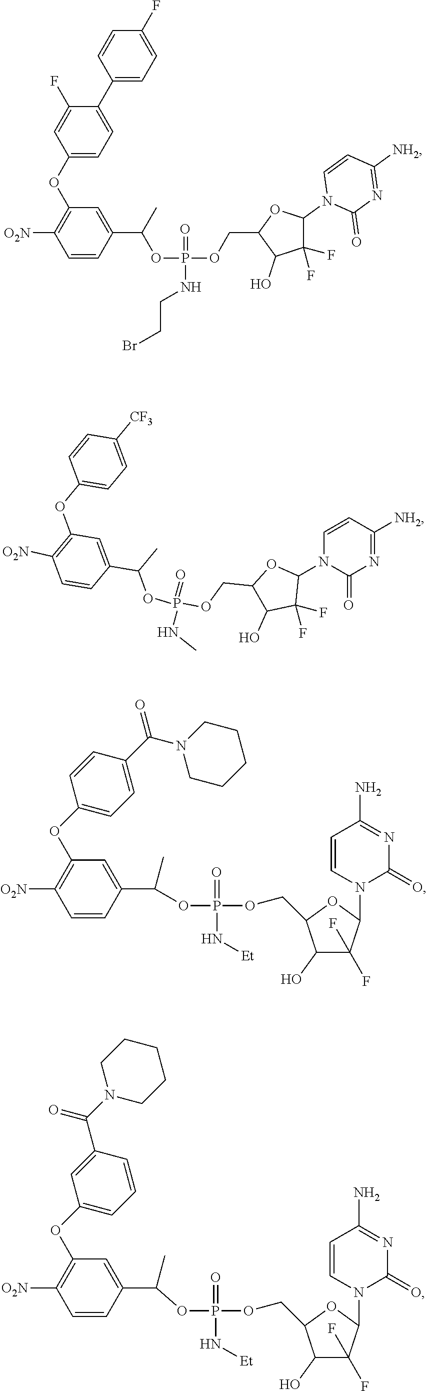

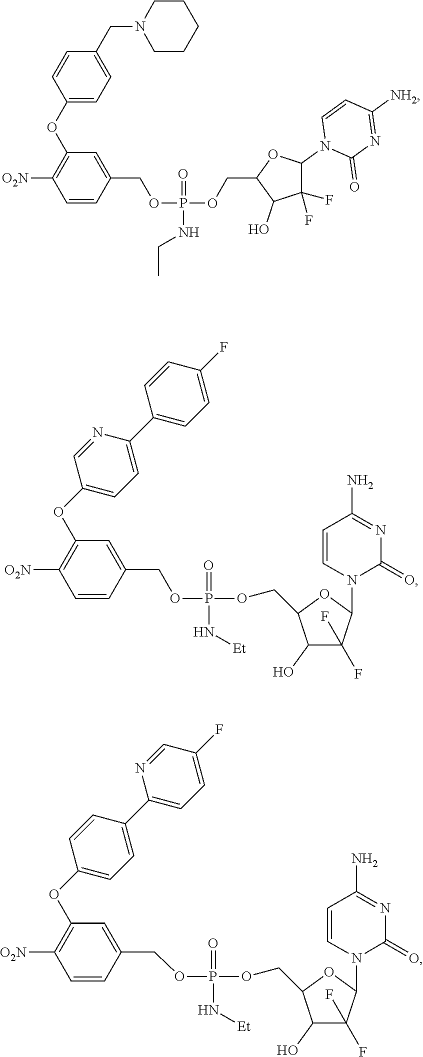

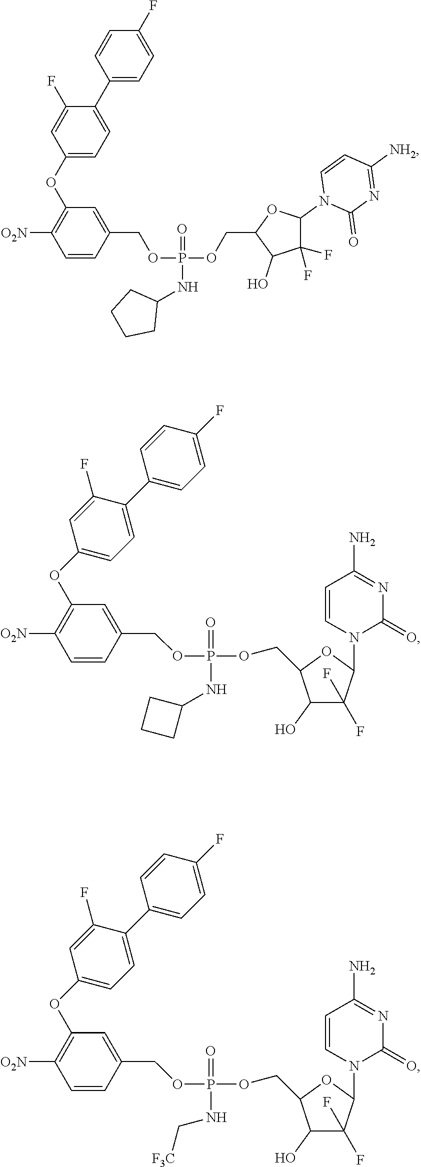

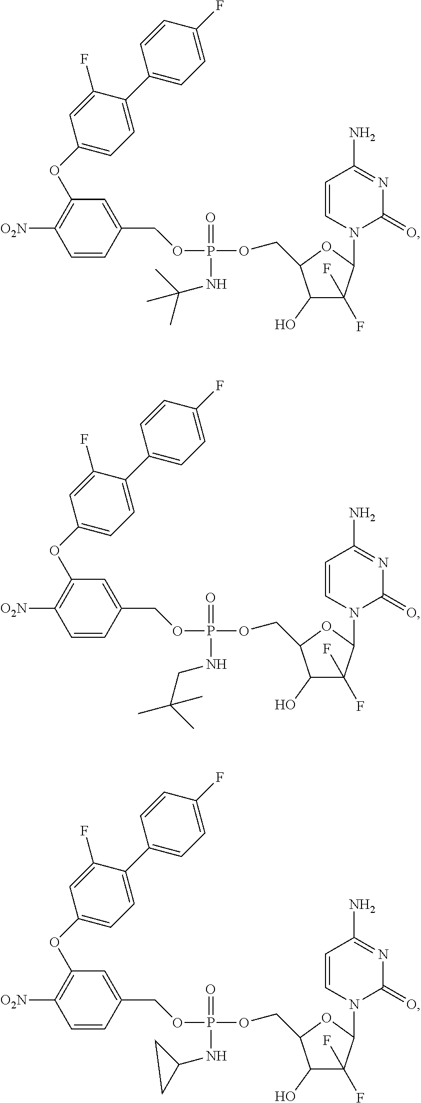

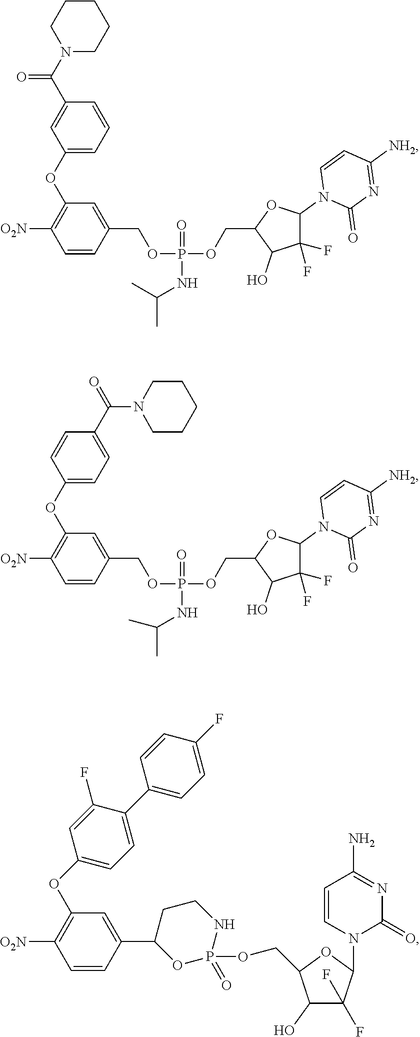

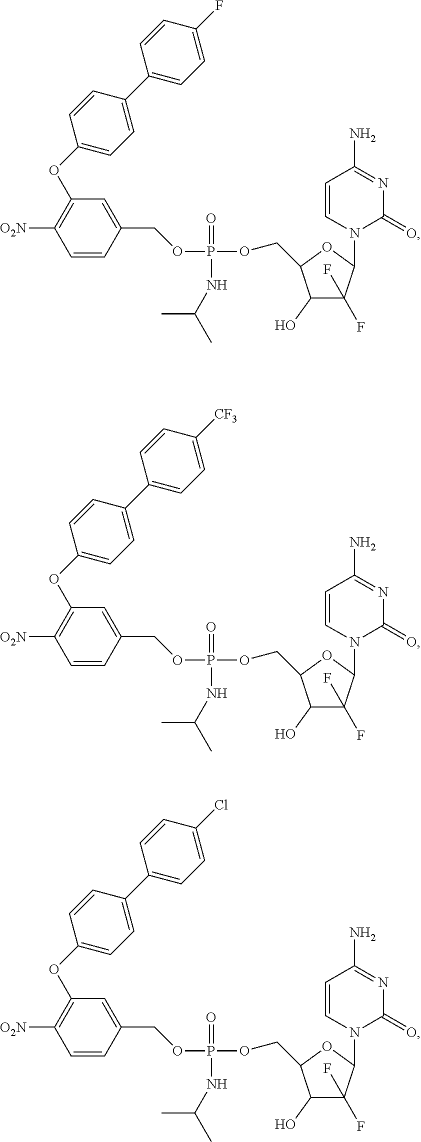

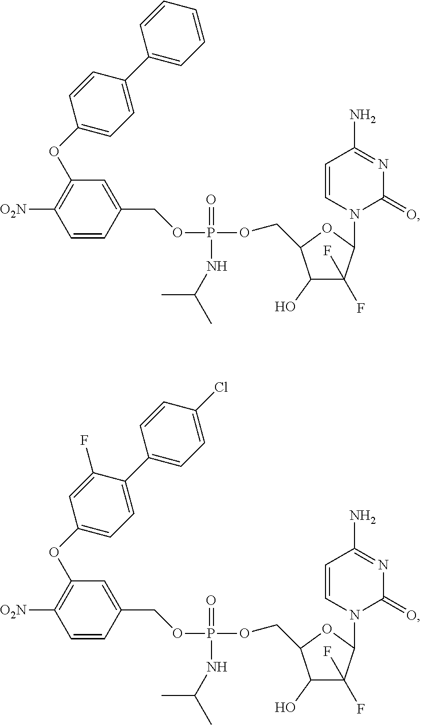

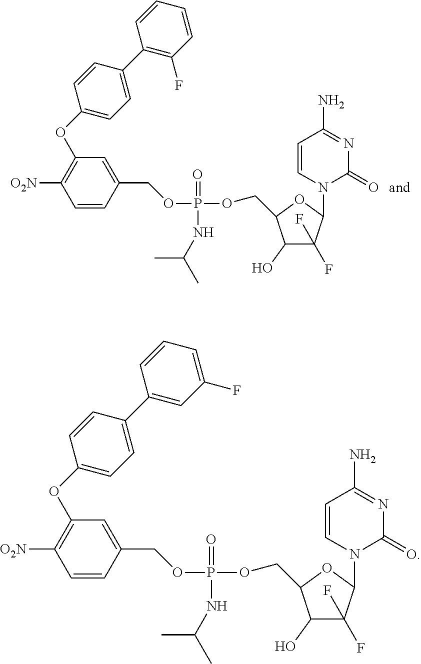

Preferably, formula (II) is selected from compounds of the following structures:

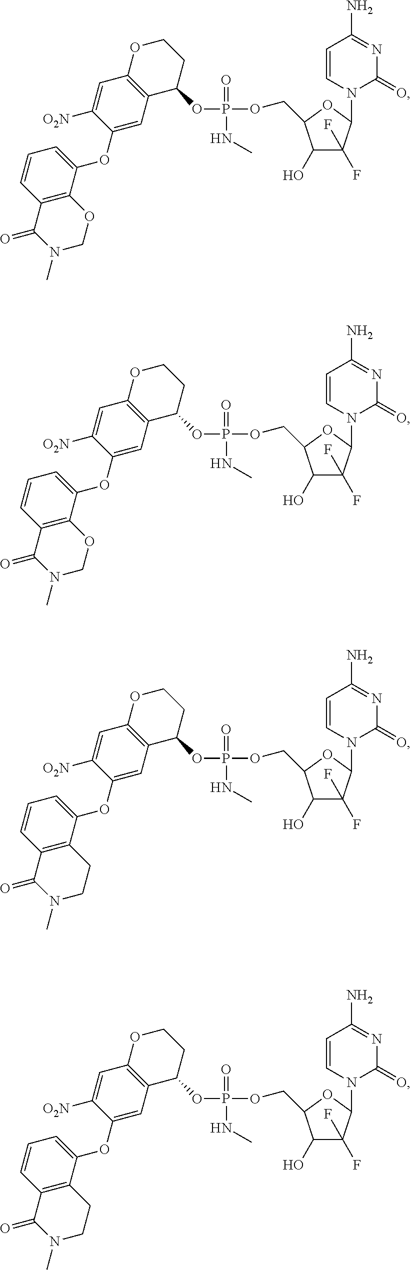

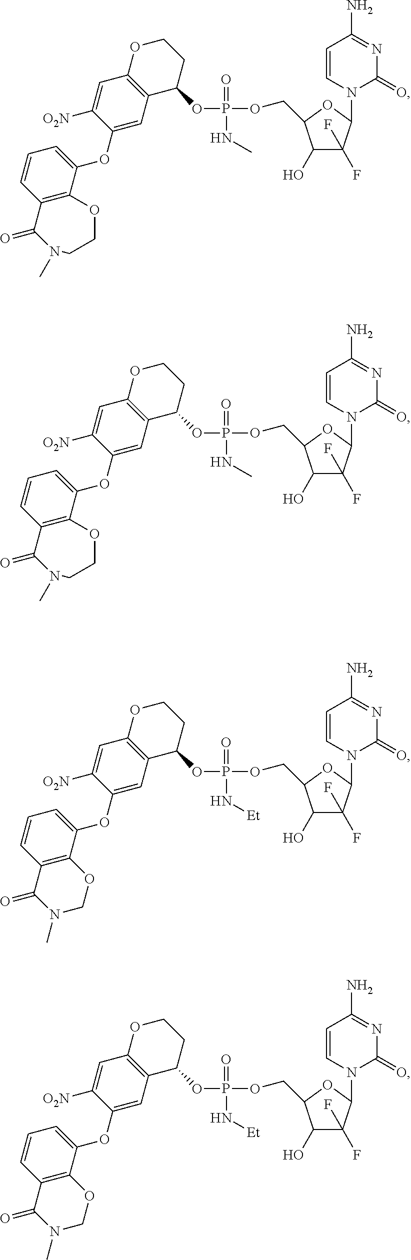

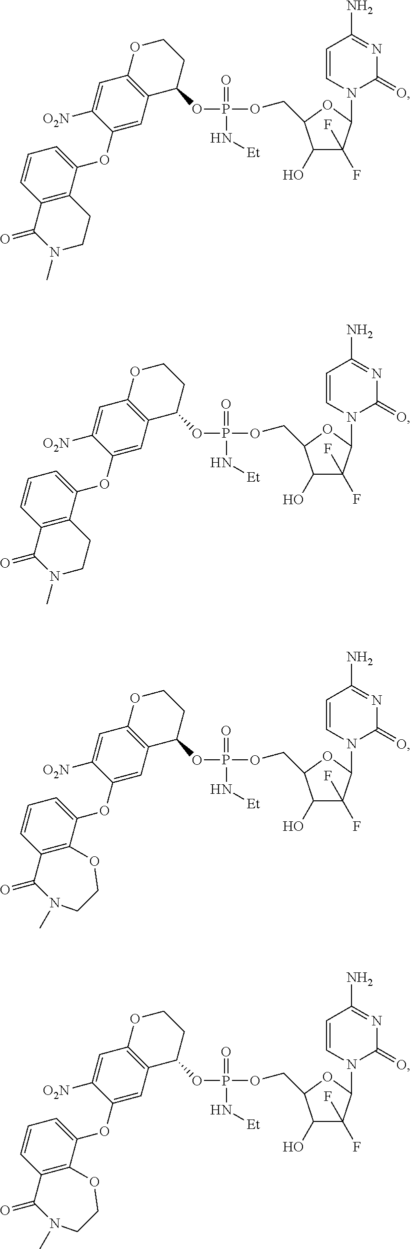

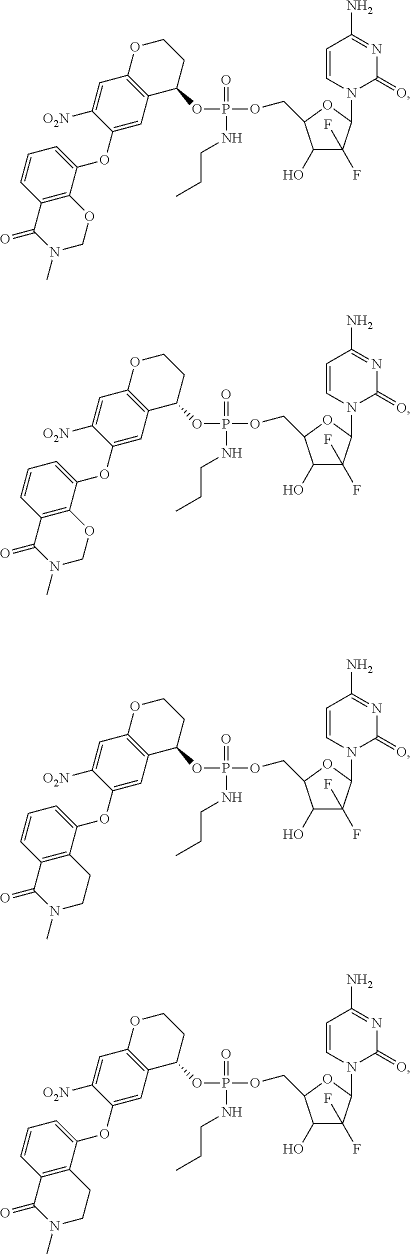

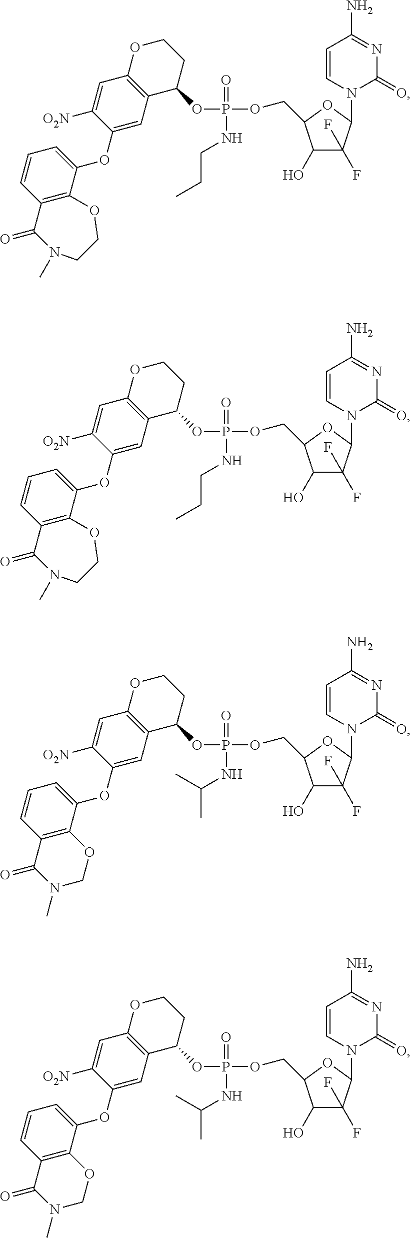

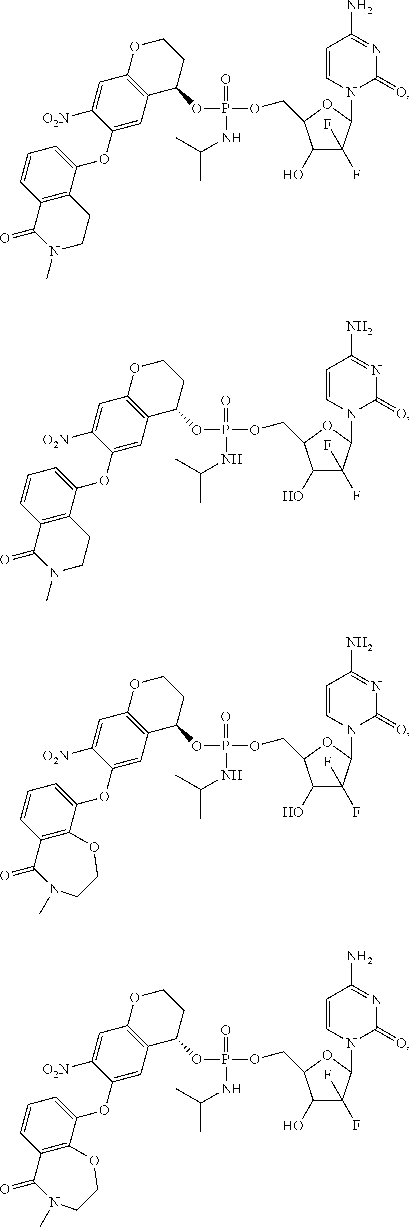

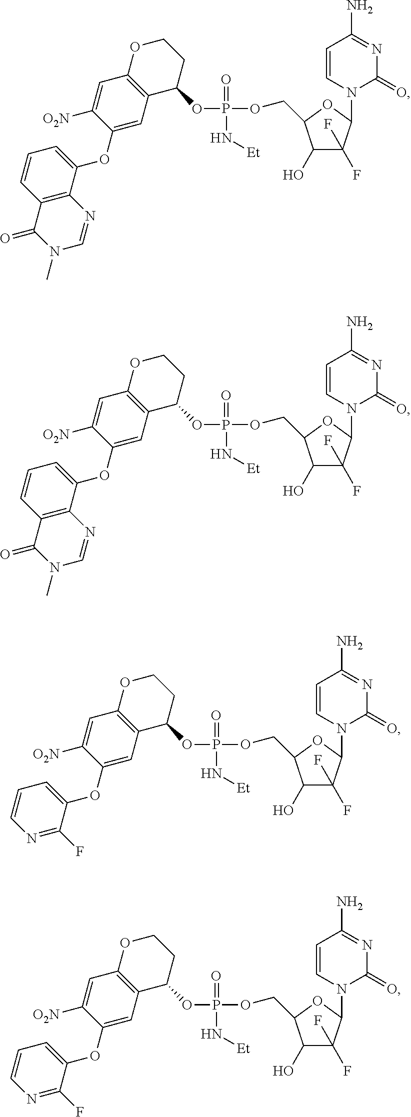

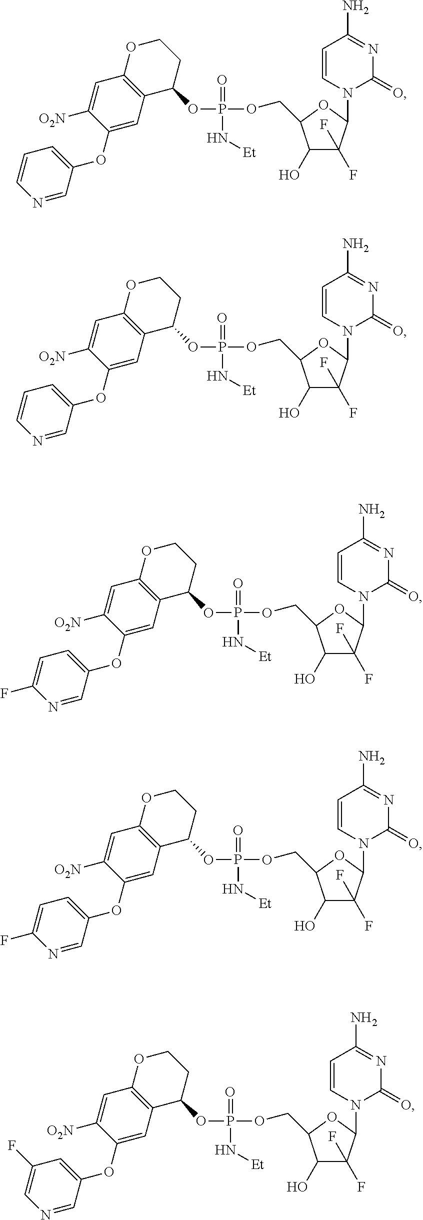

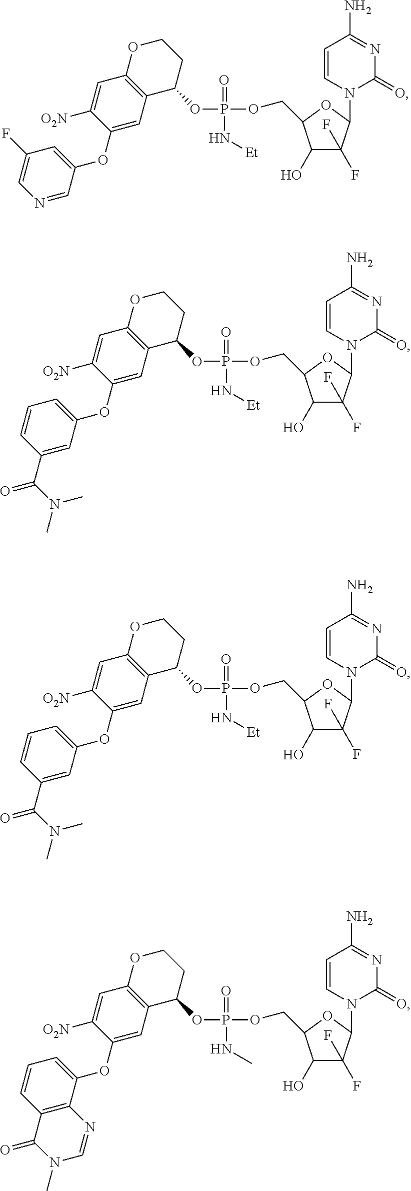

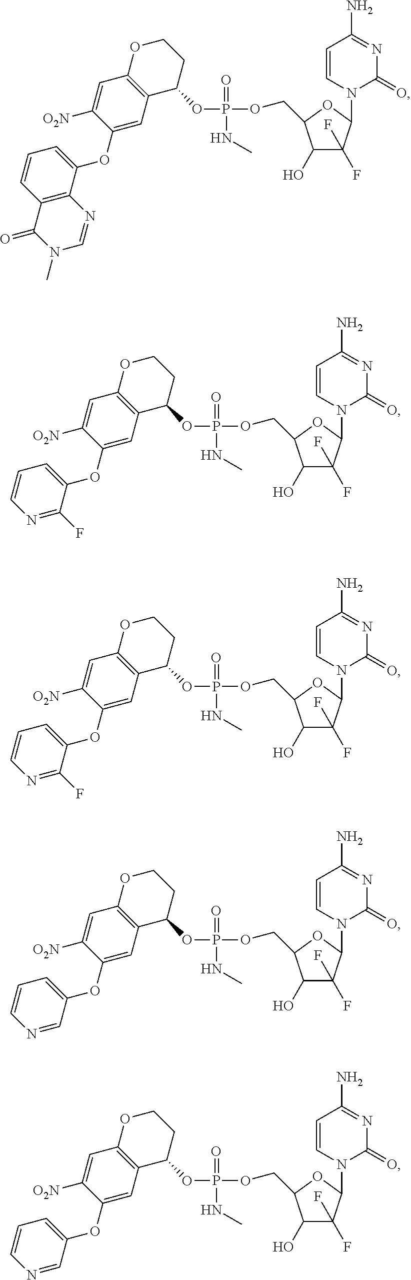

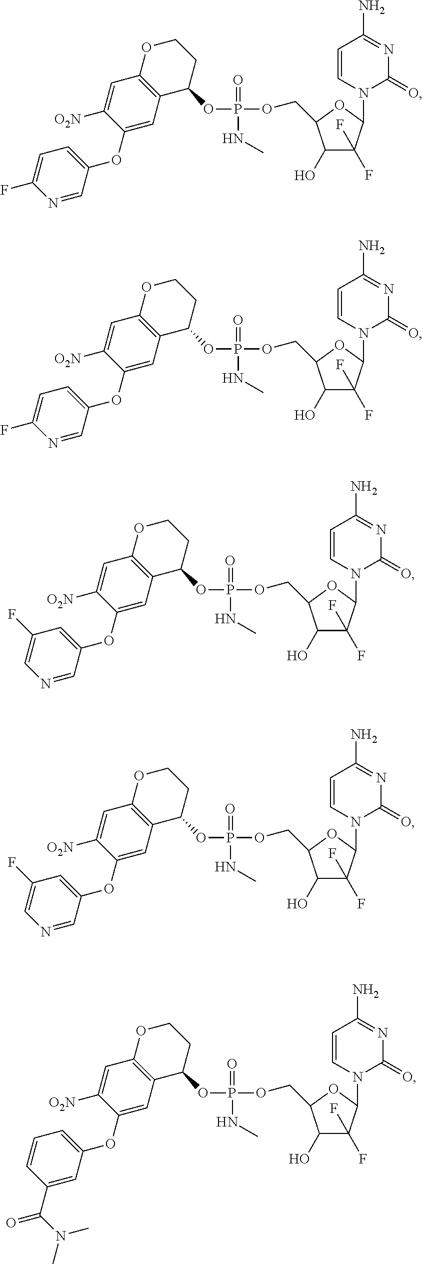

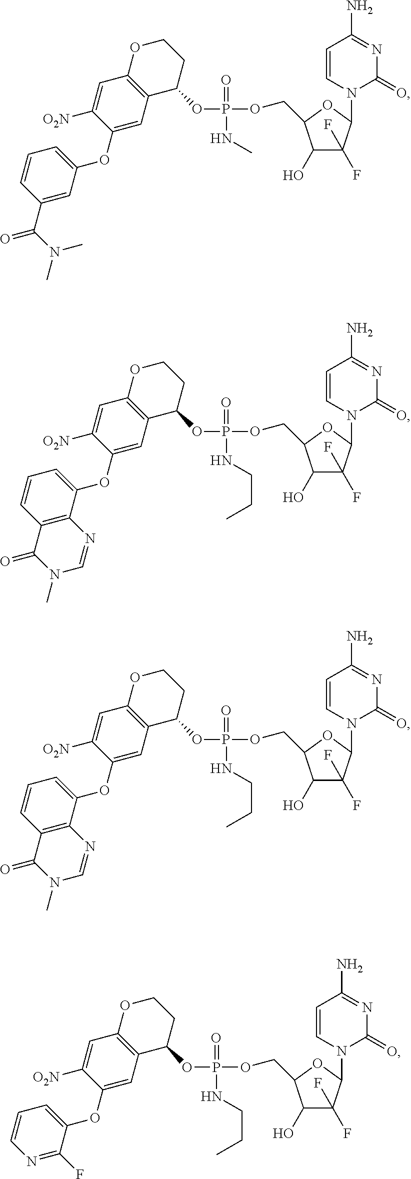

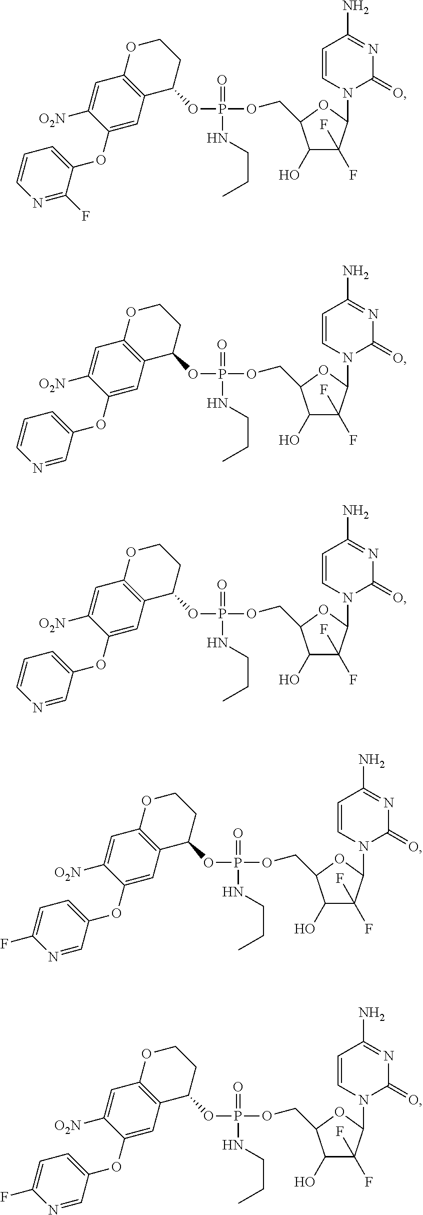

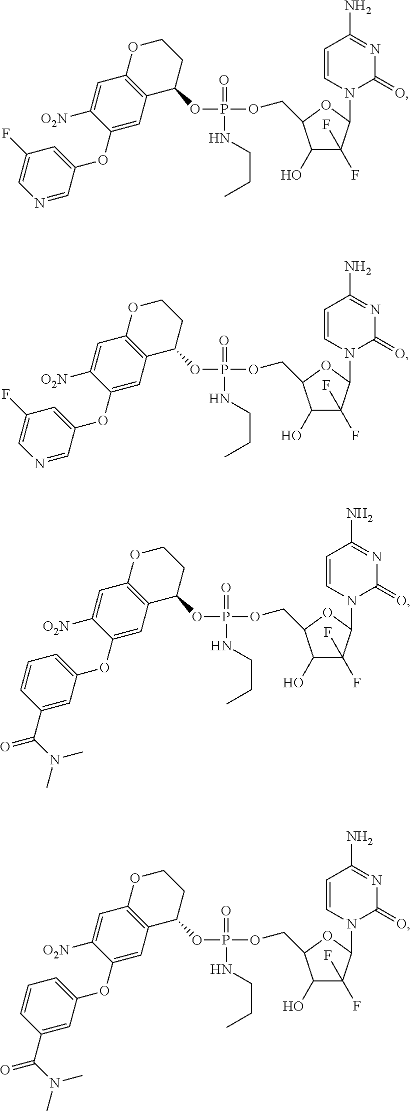

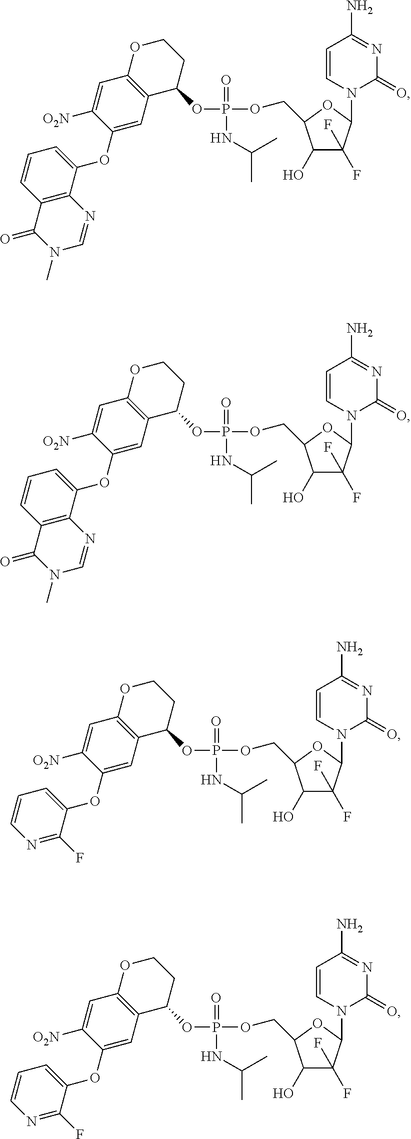

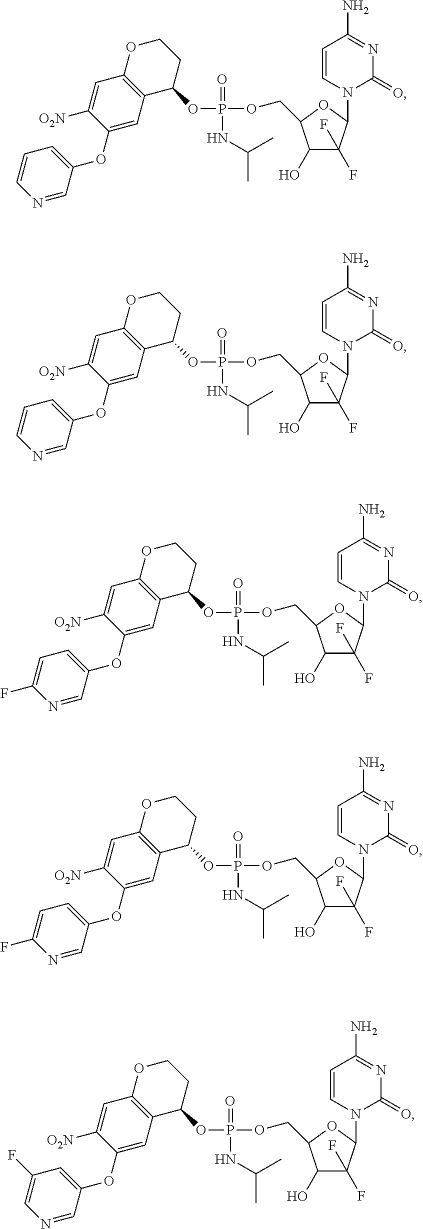

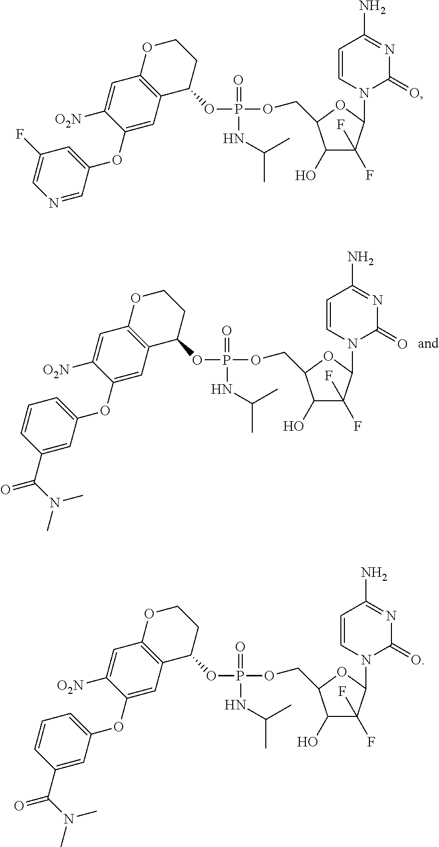

Further, the salt is a basic salt or an acid salt, and the solvate is a hydrate or alcoholate.

The compound also includes the form of their salts of formula II or formula III; that is, the present invention provides a pharmaceutically acceptable salt of the compound, wherein the salt may be a basic salt, including salts of the compound formed with inorganic bases (such as alkali metal hydroxides, alkaline earth metal hydroxides, and the like), or with organic bases (such as mono-, di-, or triethanolamine, and the like). Alternatively, the salt may be an acid salt including salts of the compound formed with inorganic acids (such as hydrochloric acid, hydrobromic acid, hydroiodic acid, nitric acid, perchloric acid, sulfuric acid, phosphoric acid, or the like), or with organic acids (such as methanesulfonic acid, trifluoromethanesulfonic acid, ethanesulfonic acid, benzenesulfonic acid, p-toluenesulfonic acid, fumaric acid, oxalic acid, maleic acid, citric acid, and the like). The selection and preparation of acceptable salts and solvates of the compound are well known in the art. For gemcitabine and derivatives thereof, preferred salts are hydrochlorides and monophosphates.

The compound described herein may further be used in the form of a solvate, wherein the solvate is a hydrate, an alcoholate and the like, and the alcoholate includes ethanolates.

The present invention also provides application of the compound as described above in the manufacture of a medicament for the treatment of tumors and cancers.

The present invention also provides a medicament or formulation containing the above compound.

Preferably, the medicament or formulation is used for the treatment of tumor and cancer diseases in patients, wherein the tumors and cancers include:
lung cancer, non-small cell lung cancer, liver cancer, pancreatic cancer, stomach cancer, bone cancer, esophagus cancer, breast cancer, prostate cancer, testicular cancer, colon cancer, ovarian cancer, bladder cancer, cervical cancer, melanoma, squamous cell carcinoma, basal cell carcinoma, adenocarcinoma, sweat gland carcinoma, sebaceous gland carcinoma, papillary carcinoma, papillary adenocarcinoma, cystic adenocarcinoma, cystic carcinoma, medullary carcinoma, bronchial carcinoma, osteocyte carcinoma, epithelial carcinoma, cholangiocarcinoma, choriocarcinoma, embryonal carcinoma, seminoma, Wilm's tumor, glioblastoma, astrocytoma, medulloblastoma, craniopharyngioma, ependymoma, pineal tumor, hemocytoblastoma, vocal cord neuroma, meningioma, neuroblastoma, optic neuroblastoma, retinoblastoma, neurofibroma, fibrosarcoma, fibroblastoma, fibroma, fibroadenoma, fibrochondroma, fibrocystoma, fibromyxoma, fibroosteoma, fibromyxosarcoma, fibropapilloma, myxosarcoma, myxocystoma, myxochondroma, myxochondrosarcoma, myxochondrofibrosarcoma, myxadenoma, myxoblastoma, liposarcoma, lipoma, lipoadenoma, lipoblastoma, lipochondroma, lipofibroma, lipoangioma, myxolipoma, chondrosarcoma, chondroma, chondromyoma, chordoma, chorioadenoma, chorioepithelioma, chorioblastoma, osteosarcoma, osteoblastoma, osteochondrofibroma, osteochondrosarcoma, osteochondroma, osteocystoma, osteodentinoma, osteofibroma, fibrosarcoma of bone, angiosarcoma, hemangioma, angiolipoma, angiochondroma, hemangioblastoma, angiokeratoma, angioglioma, angioendothelioma, angiofibroma, angiomyoma, angiolipoma, angiolymphangioma, angiolipoleiomyoma, angiomyolipoma, angiomyoneuroma, angiomyxoma, angioreticuloma, lymphangiosarcoma, lymphogranuloma, lymphangioma, lymphoma, lymphomyxoma, lymphosarcoma, lymphangiofibroma, lymphocytoma, lymphoepithelioma, lymphoblastoma, endothelioma, endothelioblastoma, synovioma, synovial sarcoma, mesothelioma, connective tissue tumor, Ewing's tumor, leiomyoma, leiomyosarcoma, leiomyoblastoma, leiomyofibroma, rhabdomyoma, rhabdomyosarcoma, rhabdomyomyxoma, acute lymphatic leukemia, acute myelogenous leukemia, anemia of chronic disease, polycythemia, lymphoma, endometrial cancer, glioma, colorectal cancer, thyroid cancer, urothelial cancer or multiple myeloma.

The present invention also provides a method for treating cancer or tumor, comprising a step of applying the aforementioned medicament or formulation; and a step of determining an AKR1C3 reductase content or expression level of cancer cells or tissues in a patient, and administering the aforementioned medicament or formulation to the patient if the measured AKR1C3 reductase content or expression level is equal to or greater than a predetermined value.

Preferably, the AKR1C3 reductase content or expression level may be measured using an AKR1C3 antibody.

The present invention also provides another method for treating cancer or tumor, comprising a step of applying the aforementioned medicament or formulation; and a step of adjusting an AKR1C3 reductase content or expression level, and
administering the aforementioned medicament or formulation to the patient when the AKR1C3 reductase content or expression level is adjusted to be equal to or greater than a predetermined value.

The AKR1C3 reductase content can be determined using methods including ELISA and IHC methods, etc.

Liquid samples such as plasma and blood can be directly tested using a commercially available human aldo-keto reductase 1C3 (AKR1C3) ELISA assay kit. Other samples are tested after being treated.

The immunohistochemical (IHC) method is suitable for testing solid tumor samples.

Detection of AKR1C3 expression level refers to the detection of corresponding mRNA expression levels. Studies show that there is a significant correlation between the AKR1C3 enzyme content and AKR1C3 RNA expression level in cancer cells of different solid tumors, and there is a significant correlation between the AKR1C3 enzyme content and AKR1C3 RNA expression level in different blood cancer cell lines. Therefore, the enzyme content can also be predicted or characterized by detecting ARKR1C3 RNA content (or expression level), thereby providing guidance on using the medicines. The q-PCR technique can be used to detect the RNA (messenger RNA, i.e. mRNA) content or expression level of AKR1C3.

Studies show that that after radiotherapy, the content of AKR1C3 enzyme in the tumor tissue of patients with head and neck cancer is increased. Therefore, it is possible to increase the expression level of AKR1C3 enzyme by irradiating the patients' tumor tissue with radioactive rays used in radiotherapy. The radioactive rays include α-, β-, γ-rays produced by radioisotopes, and X-rays, electron rays, proton beams and other particle beams produced by various X-ray therapeutic machines or accelerators.

Studies have found that certain chemical components can also promote the expression of AKR1C3 enzyme in the human body, such as kukui (scientific name: Aleurites moluccana (L.) Willd.) seed oil (Japanese patent application (Publication No. JP2021145582A) entitled PRODUCTION PROMOTER FOR AKR1C1, AKR1C2 AND AKR1C3).

This method is mainly for the situation where the content of AKR1C3 reductase in a patient is relatively low, and is performed by adjusting the content level of AKR1C3 reductase in the patient to an appropriate level through a certain adjustment treatment/administration process.

Regarding the medicament or formulation described herein, the prepared medicament contains a specific dosage range of the shown compounds or salts or solvates thereof, and/or the prepared medicament is in a specific dosage form and is administered using a specific administration method.

The medicament or formulation herein may also contain pharmaceutically acceptable auxiliaries or excipients. The medicament may be in any dosage form for clinical administration, such as tablets, suppositories, dispersible tablets, enteric-coated tablets, chewable tablets, orally disintegrating tablets, capsules, sugar-coated preparations, granules, dry powders, oral solutions, injections, lyophilized powder for injection, or infusion solutions. According to the specific dosage form and the administration method, the pharmaceutically acceptable auxiliaries or excipients in the medicament may include one or more of the following: diluents, solubilizers, disintegrants, suspensions, lubricants, binding agents, fillers, flavoring agents, sweeteners, antioxidants, surfactants, preservatives, wrapping agents, and pigments.

The medicament should be applied or administered so as to reach a “therapeutically effective amount”. The “therapeutically effective amount” refers to an amount of a drug that, when administered to a patient with cancer, will have the intended therapeutic effect, e.g., alleviation, amelioration, palliation, or elimination of one or more manifestations of cancer in the patient. A therapeutic effect does not necessarily occur by administration of one dose, and may occur only after administration of a series of doses. Thus, a therapeutically effective amount may be administered in one or more administrations.

“Treatment” of a condition or patient refers to taking steps to obtain beneficial or desired results, including clinical results. For purposes of this invention, beneficial or desired clinical results include, but are not limited to, alleviation or amelioration of one or more symptoms of cancer; diminishment of extent of disease; delay or slowing of disease progression; amelioration, palliation, or stabilization of the disease state; or other beneficial results. Treatment of cancer may, in some cases, result in partial response or stable disease.

A subject of “treating cancer or tumor” refers to any suitable species: mammals or fish, such as mammals, e.g., murines, dogs, cats, horses, or humans; preferably, the patient is a mammal, more preferably a human.

The definitions of isotopic variants or isomers in this invention are consistent with the definitions of isotopic variants and chiral isomers in Patent Application No. PCT/US2016/062114 (Publication No. WO2017087428), corresponding to Chinese Patent Application No. 2016800446081 (Publication No. CN108290911A).

Terms not explained or illustrated in detail in this application document shall be interpreted in accordance with textbooks of medicinal chemistry, organic chemistry, biochemistry, or pharmacology.

### BRIEF DESCRIPTION OF THE DRAWINGS

FIG. 1 shows the curves of inhibition rates of gemcitabine at different concentrations on H460 cancer cells in the presence or absence of AKR1C3 inhibitor AST-3021, wherein the abscissa (conc.Log(nM)) represents the logarithm value of the concentration value in nmol/L with base 10, and the ordinate (inhibition %) represents the percent inhibition rate.

FIG. 2 shows the curves of inhibition rates of compound H11 at different concentrations on H460 cancer cells in the presence or absence of AKR1C3 inhibitor AST-3021, wherein the abscissa (conc.Log(nM)) represents the logarithm value of the concentration value in nmol/L with base 10, and the ordinate (inhibition %) represents the percent inhibition rate.

FIG. 3 shows the curves of inhibition rates of compound H11 at different concentrations on HepG2 cancer cells in the presence or absence of AKR1C3 inhibitor AST-3021, wherein the abscissa (conc.Log(nM)) represents the logarithm value of the concentration value in nmol/L with base 10, and the ordinate (inhibition %) represents the percent inhibition rate.

FIG. 4 shows the curves of tumor volume changes with days in the in vivo pharmacodynamic experiment of compound H11 and gemcitabine in pancreatic cancer PA1222 model.

FIG. 5 shows the curves of weight change rates of mice with days in the in vivo pharmacodynamic experiment of compound H11 and gemcitabine in pancreatic cancer PA1222 model.

### DETAILED DESCRIPTION OF THE INVENTION

The present invention will be described below with reference to specific examples. Those skilled in the art can understand that these examples are only used for describing the invention and do not in any way limit its scope.

The experimental methods in the following examples are all conventional methods unless otherwise specified. The raw materials of the medicaments, the reagents and the like used in the following examples are all commercially available products unless otherwise specified.

### I. Test of Inhibition of H460 Cancer Cells

In vitro human tumor cell line cytotoxicity assay was used.

In vitro proliferation data on the H460 non-small cell lung cancer human tumor cell line (cell line which highly expresses AKR1C3) is reported below in the compound table.

IC₅₀ values were obtained with the following steps:

Specifically, exponentially growing cells were seeded at a density of 2×10³ cells/100 μL/well in a 96-well plate and incubated at 37° C. in 5% CO₂, 95% air and 100% relative humidity for 24 hours. The test compound was administered alone: 24 hours after cell plating, the compound acted alone, 99 μL of growth medium per well was added, and 1 μL of the compound prepared at 200× was added, and the plate was shaken gently to ensure even mixing, then placed into an incubator of 37° C., 5% CO₂. After further cultivation for 72 hours, the number of live cells was detected using CTG. The CTG detection method was as follows: the cell plate to be tested was placed at room temperature to equilibrate for 30 minutes, and 100 μL of medium per well was discarded. 25 μL of CTG reagent (CellTiter-Glo® kit) per well was added, and the plate was placed in a high-speed shaker to oscillate for 2 minutes, and placed at room temperature in the dark for 30 minutes. The chemiluminescence signal value was read using a multimode microplate reader, with a read time of 1000 ms. The drug concentration resulting in growth inhibition of 50% (IC₅₀) was calculated using computer software, and the results were listed in Table 1, providing values corresponding to IC₅₀ (nM).

Similarly, in order to further verify that the compounds were activated by human AKR1C3 (aldosterone reductase family 1 member C3), the effect of some of the compounds on the proliferation of H460 cancer cells was tested with pretreatment with a specific AKR1C3 enzyme inhibitor AST-3021 (3 μM). The detailed experimental procedure was as follows: 24 hours after cell plating, 98 μL of growth medium per well was added, and 1 μL of 200×compound AST-3021 was added to each well, and the plate was shaken gently to ensure even mixing, and then placed into an incubator; after 2 hours, 1 μL of the test compound prepared at 200× was added, and the plate was shaken gently to ensure even mixing and then laced into an incubator of 37° C., 5% CO₂. The inhibitor used was compound 36, i.e.

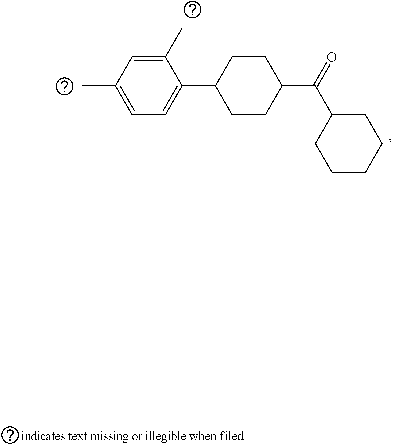 [SMILES: Cc1ccc(C2CCC(C(=O)C3CCCCC3)CC2)c(C)c1]

in Flanagan et al., Bioorganic and Medicinal Chemistry (2014), page 962-977, and is referred to as AST-3021 in this invention. The values measured in this case are values corresponding to +AST-3021 IC₅₀ (nM) in Table 1.

The value corresponding to +AST-3021 IC₅₀ (nM) divided by the value corresponding to IC₅₀ (nM) is the specific activation ratio. The relative magnitude of the specific activation ratio can be used to evaluate whether this compound is a cytotoxic compound activated by human AKR1C3 (aldosterone reductase family 1 member C3): when the specific activation ratio is less than 1, it is considered that the compound is not activated by human AKR1C3 (aldosterone reductase family 1 member C3); when the specific activation ratio is greater than 1, it is considered that the compound is activated by human AKR1C3 (aldosterone reductase family 1 member C3), and the higher the value, the higher the activation degree and the selectivity of AKR1C3.

TABLE 1
Comparison data of IC₅₀ values and specific activation ratios (+AST-3021 IC₅₀/IC₅₀) of various compounds ±AST-3021

| | | | | | | | |
|---|---|---|---|---|---|---|---|
| | | | +AST- | | | | |
| | | | 3021 | Comparative | | | |
| Compound | | IC₅₀ | IC₅₀ | compound | Comparative compound | IC₅₀ | +AST-3021 |
| No. | Compound structure | (nM) | (nM) | No. | structure | (nM) | IC₅₀ (nM) |

| H1 | 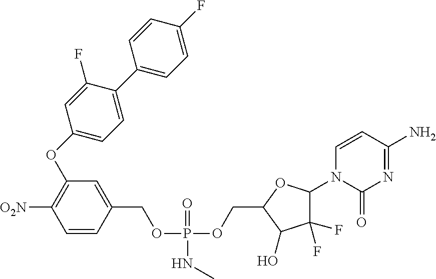 [SMILES: CNP(=O)(OCc1ccc([N+](=O)[O-])c(Oc2ccc(-c3ccc(F)cc3)c(F)c2)c1)OCC1OC(n2ccc(N)nc2=O)C(F)(F)C1O] | 29.4 711.7 specific activation ratio 24.2 | / | / | |
|---|---|---|---|---|---|
| H2 | 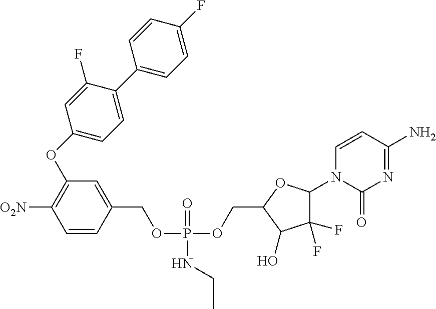 [SMILES: CCNP(=O)(OCc1ccc([N+](=O)[O-])c(Oc2ccc(-c3ccc(F)cc3)c(F)c2)c1)OCC1OC(n2ccc(N)nc2=O)C(F)(F)C1O] | 19.89 951 specific activation ratio 47.8 | D1 | 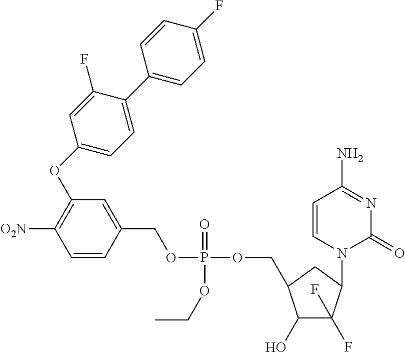 | 774.1 1223 specific activation ratio 1.6 |
| | | | D2 | 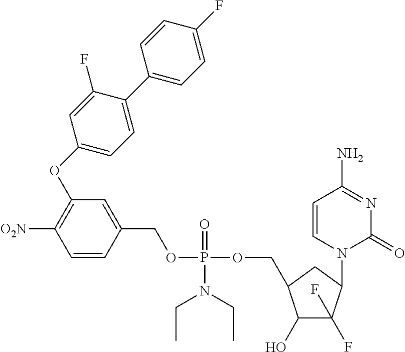 | 96.87 4371 specific activation ratio 45.1 |
| | | | D3 | 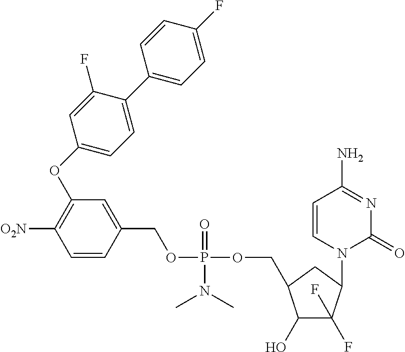 | 19.9 787.3 specific activation ratio 39.6 |
| H3 | 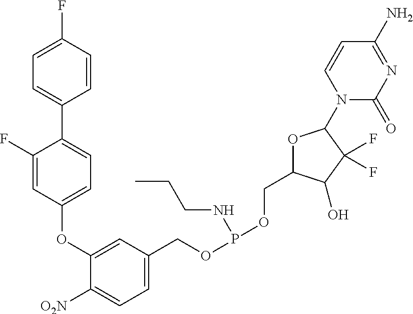 [SMILES: CCCNP(OCc1ccc([N+](=O)[O-])c(Oc2ccc(-c3ccc(F)cc3)c(F)c2)c1)OCC1OC(n2ccc(N)nc2=O)C(F)(F)C1O] | 32.73 1491 specific activation ratio 45.6 | D4 | 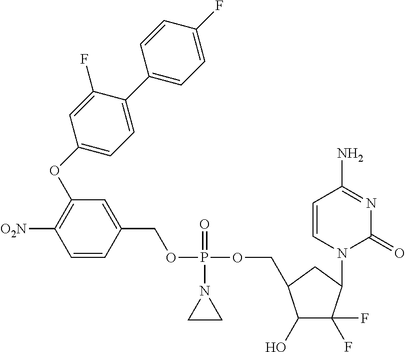 [SMILES: Nc1ccn(C2CC(COP(=O)(OCc3ccc([N+](=O)[O-])c(Oc4ccc(-c5ccc(F)cc5)c(F)c4)c3)N3CC3)C(O)C2(F)F)c(=O)n1] | 17.73 19.82 specific activation ratio 1.1 |
| H4 | 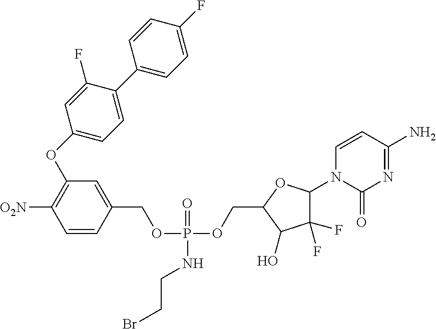 [SMILES: Nc1ccn(C2OC(COP(=O)(NCCBr)OCc3ccc([N+](=O)[O-])c(Oc4ccc(-c5ccc(F)cc5)c(F)c4)c3)C(O)C2(F)F)c(=O)n1] | 5.15 31.68 specific activation ratio 6.2 | D4 | 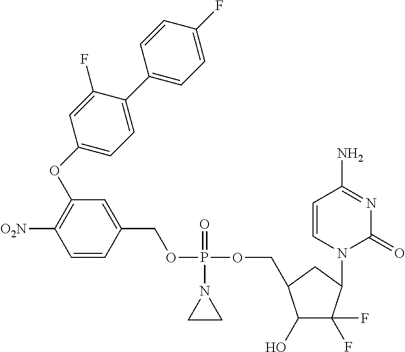 [SMILES: Nc1ccn(C2CC(COP(=O)(OCc3ccc([N+](=O)[O-])c(Oc4ccc(-c5ccc(F)cc5)c(F)c4)c3)N3CC3)C(O)C2(F)F)c(=O)n1] | 17.73 19.81 specific activation ratio 1.1 |
| H5 | 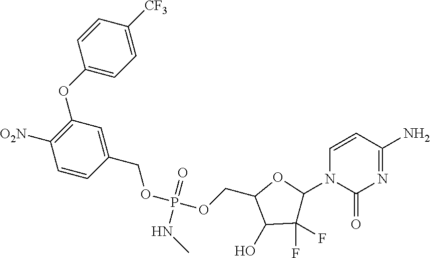 [SMILES: CNP(=O)(OCc1ccc([N+](=O)[O-])c(Oc2ccc(C(F)(F)F)cc2)c1)OCC1OC(n2ccc(N)nc2=O)C(F)(F)C1O] | 117.4 498.7 specific activation ratio 4.2 | / | / | |
| H6 | 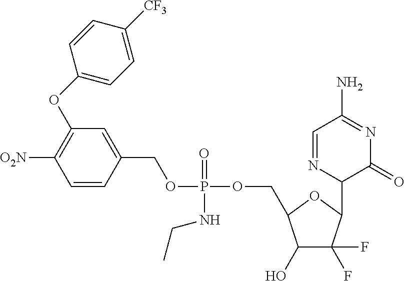 [SMILES: CCNP(=O)(OCc1ccc([N+](=O)[O-])c(Oc2ccc(C(F)(F)F)cc2)c1)OCC1OC(C2N=CC(N)=NC2=O)C(F)(F)C1O] | 178.2 2304 specific activation ratio 12.9 | D5 | 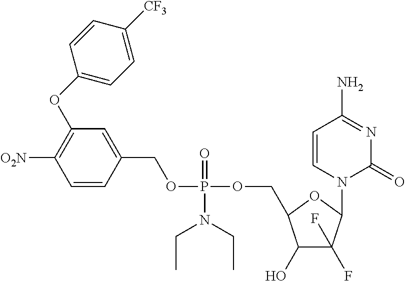 | 167.2 1474 specific activation ratio 8.8 |
| H7 | 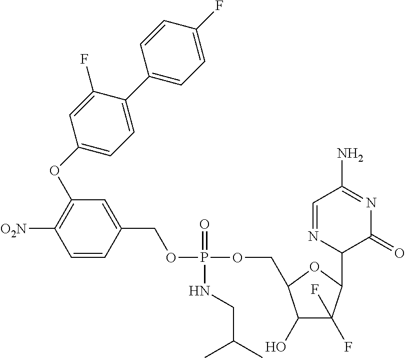 | 26.78 1642 specific activation ratio 61.3 | D2 | 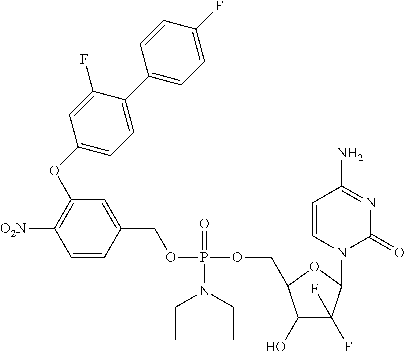 | 96.87 4371 specific activation ratio 45.1 |
| H8 | 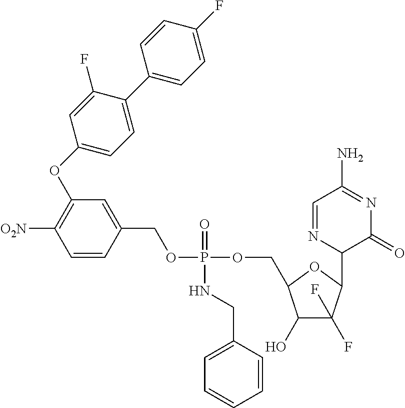 | 67.47 3022 specific activation ratio 44.8 | D6 | 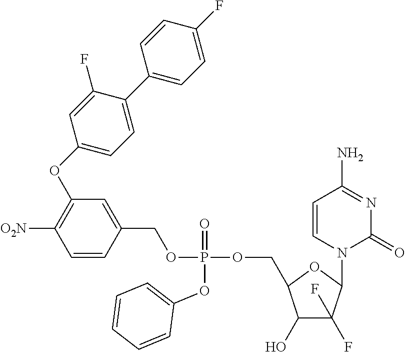 | 20.57 11.17 specific activation ratio 0.54 |
| H9 | 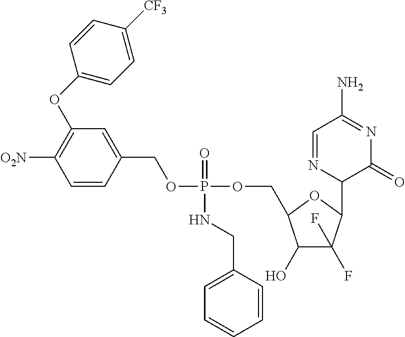 | 78.15 1811 specific activation ratio 23.2 | D7 | 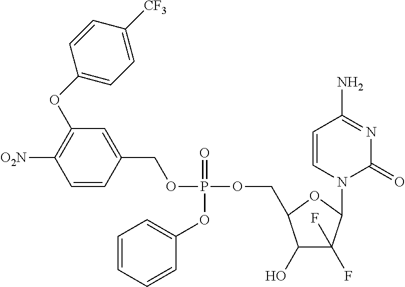 | 50.26 38.91 specific activation ratio 0.77 |
| H10 | 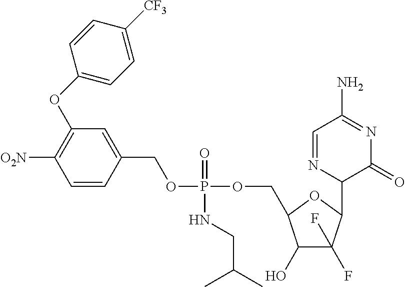 | 104.60 2902 specific activation ratio 27.7 | D5 | 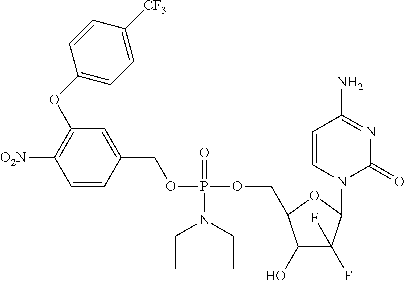 | 167.2 1474 specific activation ratio 8.8 |
| H11 | 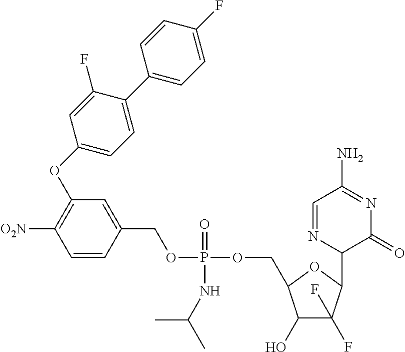 | 15.9 1476 specific activation ratio 92.8 | D8 | 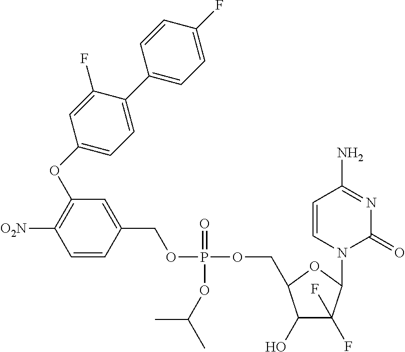 | / / / |
| H12 | 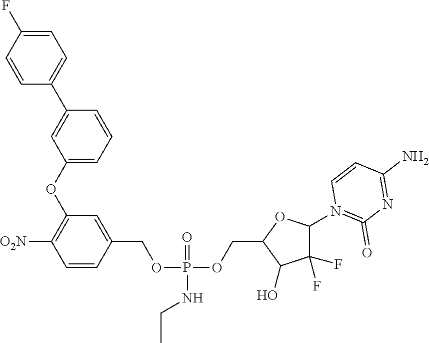 | 73.84 716.9 specific activation ratio 9.7 | / | / | |
| H13 | 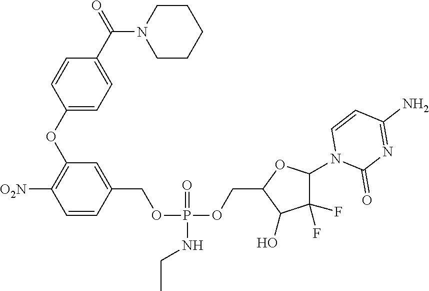 [SMILES: CCNP(=O)(OCc1ccc([N+](=O)[O-])c(Oc2ccc(C(=O)N3CCCCC3)cc2)c1)OCC1OC(n2ccc(N)nc2=O)C(F)(F)C1O] | 392.90 3677 specific activation ratio 9.4 | / | / | |
| H14 | 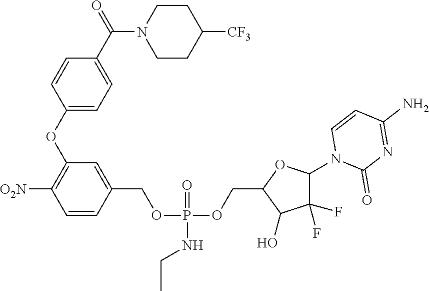 [SMILES: CCNP(=O)(OCc1ccc([N+](=O)[O-])c(Oc2ccc(C(=O)N3CCC(C(F)(F)F)CC3)cc2)c1)OCC1OC(n2ccc(N)nc2=O)C(F)(F)C1O] | 1772 3298 specific activation ratio 1.9 | / | / | |
| H15 | 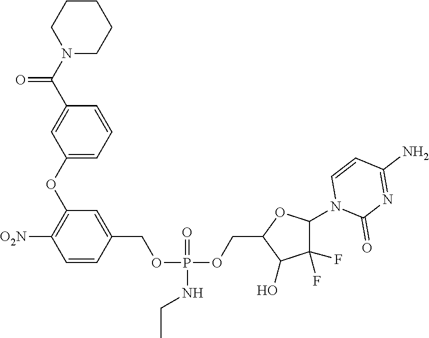 [SMILES: CCNP(=O)(OCc1ccc([N+](=O)[O-])c(Oc2cccc(C(=O)N3CCCCC3)c2)c1)OCC1OC(n2ccc(N)nc2=O)C(F)(F)C1O] | 485.8 2416 specific activation ratio 5.0 | / | / | |
| H16 | 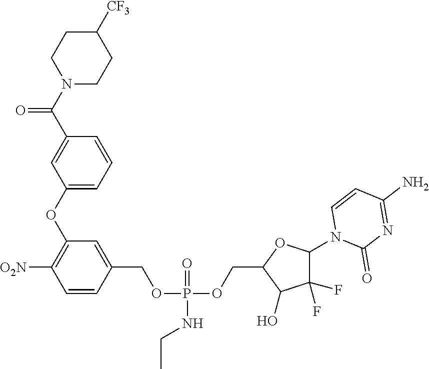 [SMILES: CCNP(=O)(OCc1ccc([N+](=O)[O-])c(Oc2cccc(C(=O)N3CCC(C(F)(F)F)CC3)c2)c1)OCC1OC(n2ccc(N)nc2=O)C(F)(F)C1O] | 376.9 3373 specific activation ratio 8.9 | / | / | |
| H17 | 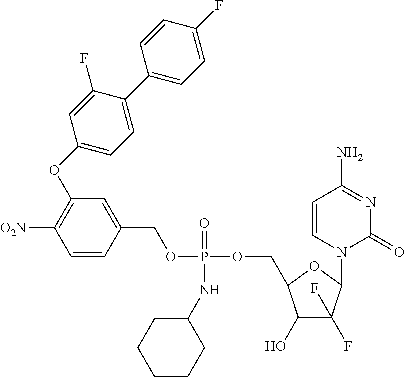 | 396.9 10815 specific activation ratio 27.2 | / | / | |
| H18 | 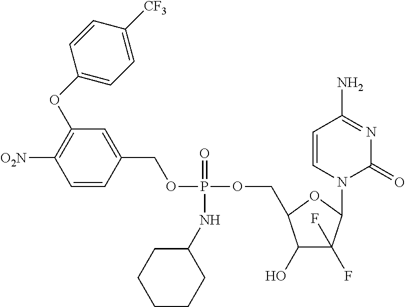 | 460.8 1700 specific activation ratio 3.7 | / | / | |
| H19 | 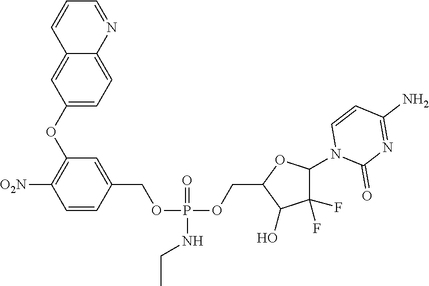 [SMILES: CCNP(=O)(OCc1ccc([N+](=O)[O-])c(Oc2ccc3ncccc3c2)c1)OCC1OC(n2ccc(N)nc2=O)C(F)(F)C1O] | 150.1 510.3 specific activation ratio 3.4 | / | / | |
| H20 | 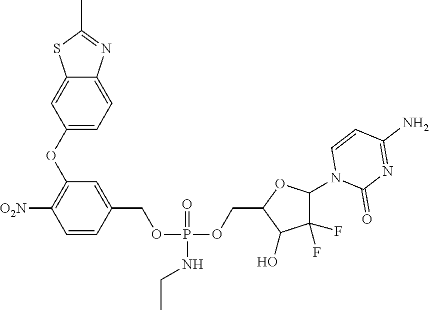 [SMILES: CCNP(=O)(OCc1ccc([N+](=O)[O-])c(Oc2ccc3nc(C)sc3c2)c1)OCC1OC(n2ccc(N)nc2=O)C(F)(F)C1O] | 231.5 1432 specific activation ratio 6.2 | / | / | |
| H21 | 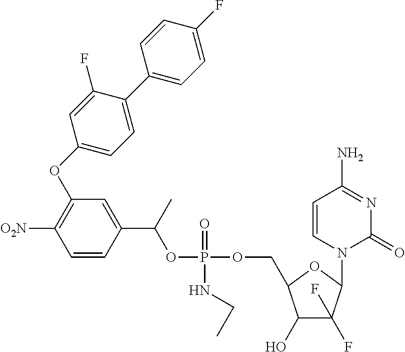 | 126.6 4412 specific activation ratio 34.8 | D9 | 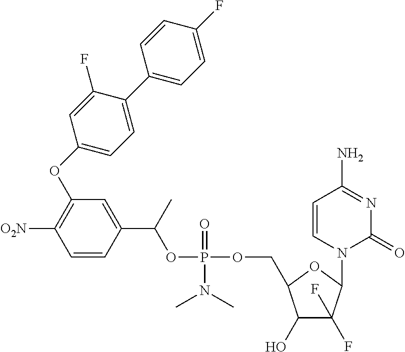 | 67.8 3830 specific activation ratio 56.5 |
| H22 | 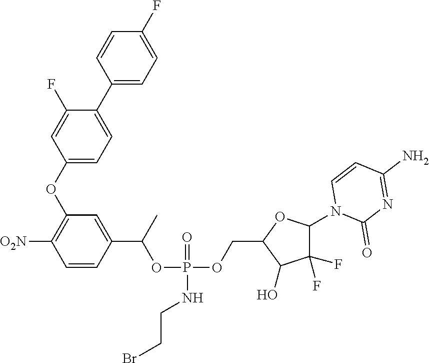 [SMILES: CC(OP(=O)(NCCBr)OCC1OC(n2ccc(N)nc2=O)C(F)(F)C1O)c1ccc([N+](=O)[O-])c(Oc2ccc(-c3ccc(F)cc3)c(F)c2)c1] | 123.8 2373 specific activation ratio 19.2 | / | / | |
| H23 | 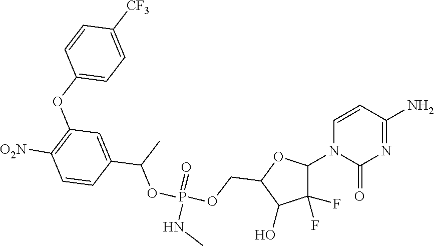 [SMILES: CNP(=O)(OCC1OC(n2ccc(N)nc2=O)C(F)(F)C1O)OC(C)c1ccc([N+](=O)[O-])c(Oc2ccc(C(F)(F)F)cc2)c1] | 379 11306 specific activation ratio 29.8 | / | / | |
| H24 | 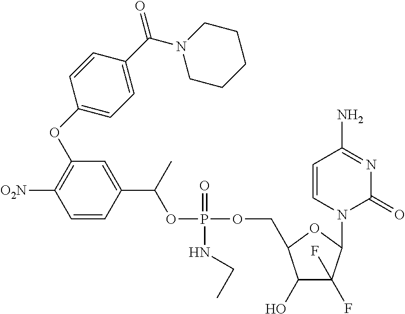 | 1124 21445 specific activation ratio 19.1 | / | / | |
| H25 | 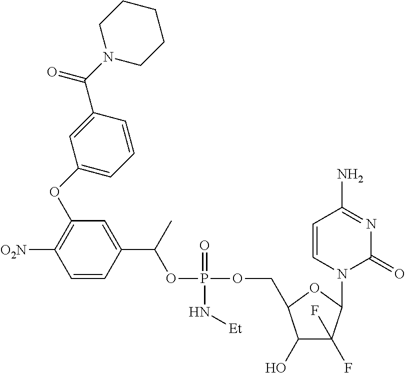 | 514.7 4935 specific activation ratio 9.6 | / | / | |
| H26 | 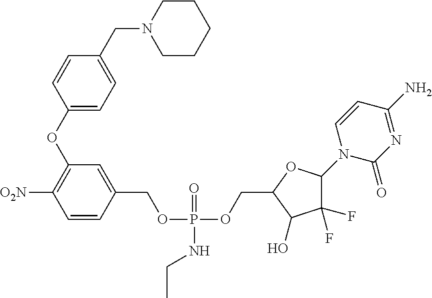 [SMILES: CCNP(=O)(OCc1ccc([N+](=O)[O-])c(Oc2ccc(CN3CCCCC3)cc2)c1)OCC1OC(n2ccc(N)nc2=O)C(F)(F)C1O] | / / / | / | / | |
| H27 | 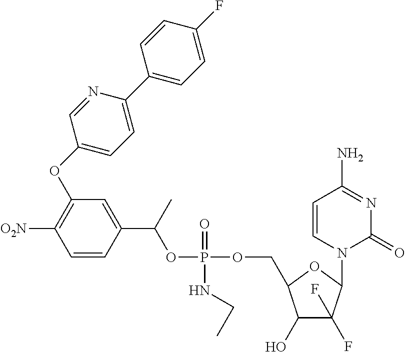 | 95.34 4319 specific activation ratio 45.3 | / | / | |
| H28 | 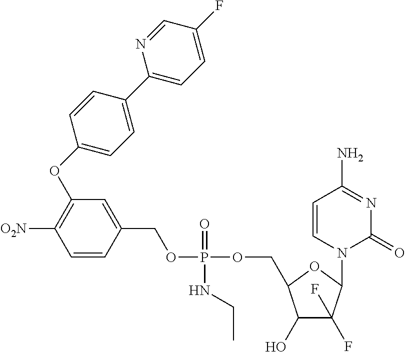 | 233.3 4763 specific activation ratio 20.4 | / | / | |
| H29 | 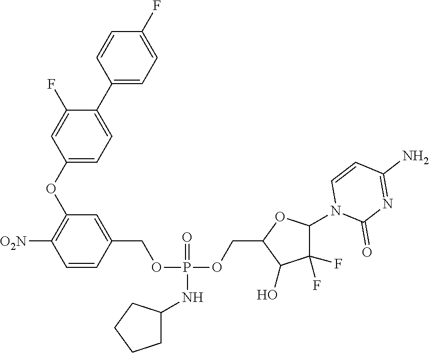 [SMILES: Nc1ccn(C2OC(COP(=O)(NC3CCCC3)OCc3ccc([N+](=O)[O-])c(Oc4ccc(-c5ccc(F)cc5)c(F)c4)c3)C(O)C2(F)F)c(=O)n1] | 82.25 3880 specific activation ratio 47.17 | / | / | |
| H30 | 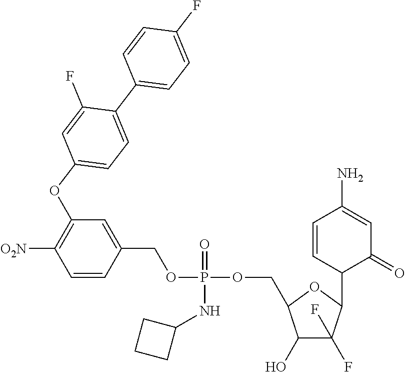 | 52.73 1371 specific activation ratio 26.0 | / | / | |
| H31 | 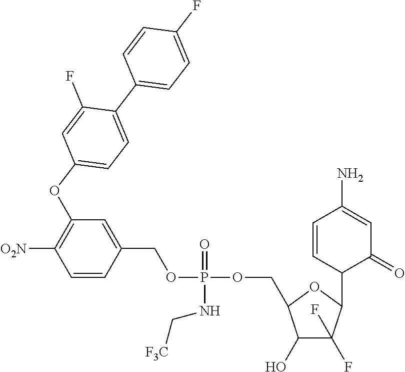 | 763.6 5288 specific activation ratio 6.9 | / | / | |
| H32 | 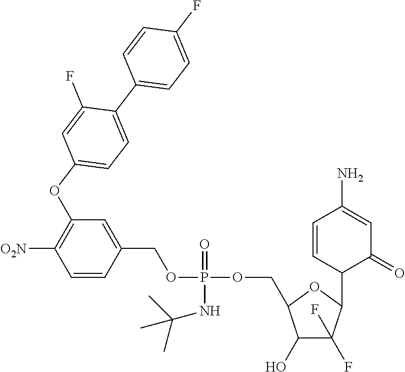 | 74.39 2582 specific activation ratio 34.7 | / | / | |
| H33 | 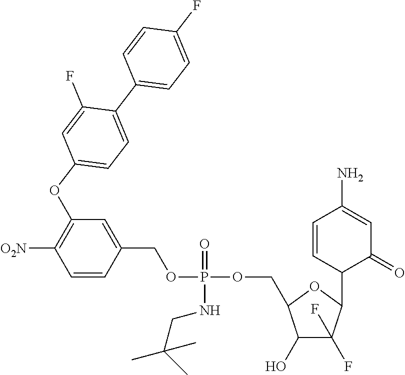 | 230.6 8603 specific activation ratio 37.3 | / | / | |
| H34 | 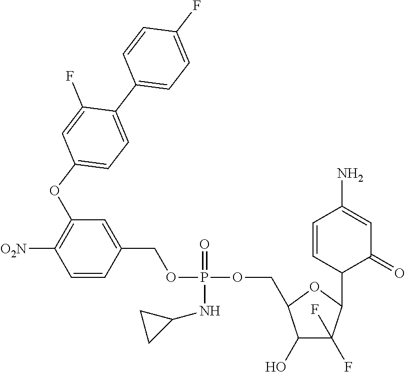 | 55.63 1038 specific activation ratio 18.7 | / | / | |
| H35 | 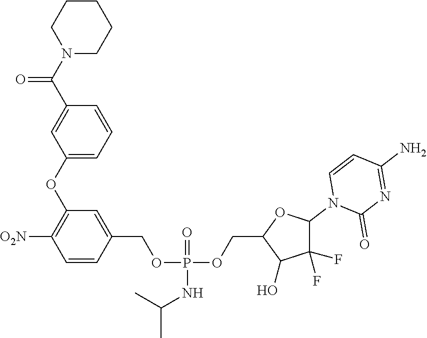 [SMILES: CC(C)NP(=O)(OCc1ccc([N+](=O)[O-])c(Oc2cccc(C(=O)N3CCCCC3)c2)c1)OCC1OC(n2ccc(N)nc2=O)C(F)(F)C1O] | 453.6 3898 specific activation ratio 8.6 | / | / | |
| H36 | 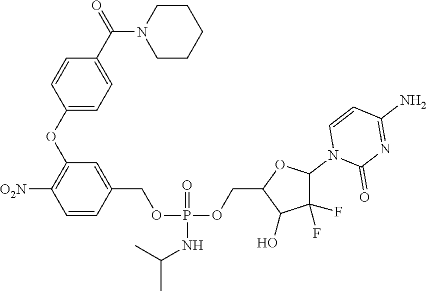 [SMILES: CC(C)NP(=O)(OCc1ccc([N+](=O)[O-])c(Oc2ccc(C(=O)N3CCCCC3)cc2)c1)OCC1OC(n2ccc(N)nc2=O)C(F)(F)C1O] | 381.8 1171 specific activation ratio 3.1 | / | / | |
| H37 | 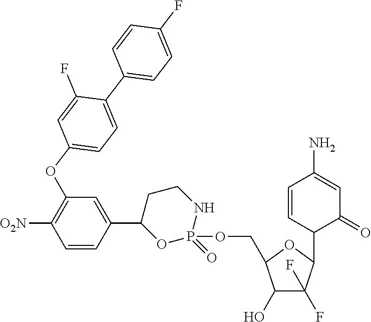 | / / / | D10 | 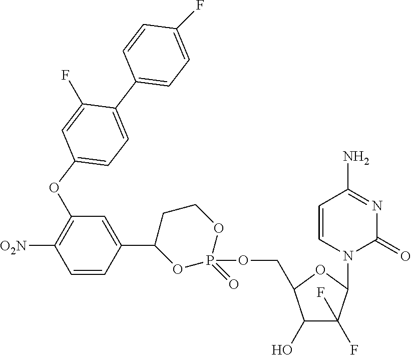 | / / / |

| | | | | |
|---|---|---|---|---|
| H38 | 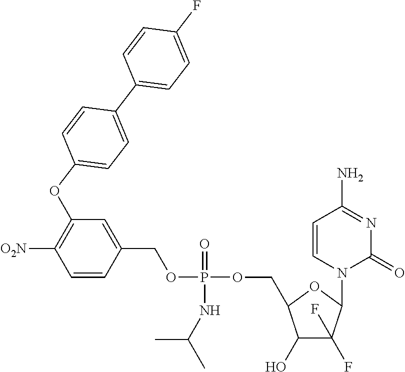 | 65.17 33080 specific activation ratio 507.6 | / | / |
| H39 | 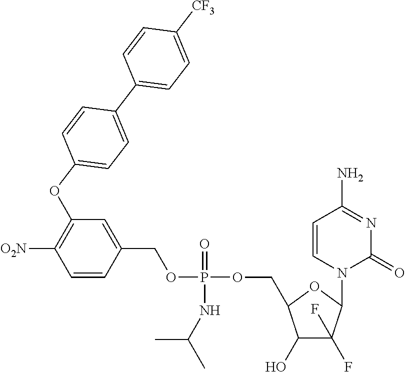 | 2183 27739 specific activation ratio 12.7 | / | / |
| H40 | 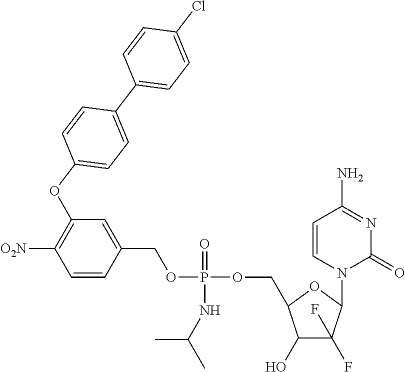 | 194.4 16896 specific activation ratio 86.9 | / | / |
| H41 | 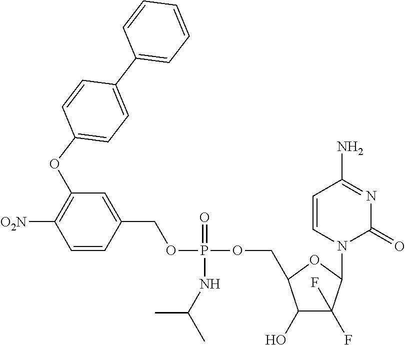 | 52.29 233.06 specific activation ratio 445.7 | / | / |
| H42 | 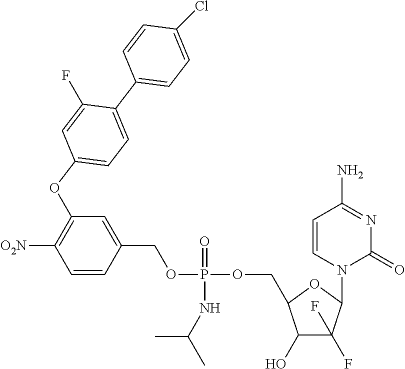 | 57.12 19047 specific activation ratio 333.5 | / | / |
| H43 | 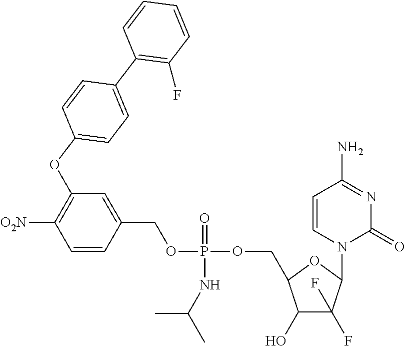 | 59.96 15455 specific activation ratio 257.8 | / | / |
| H44 | 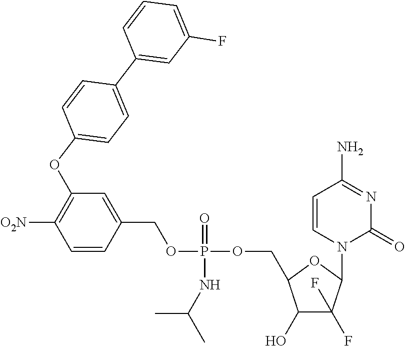 | 51.33 17251 specific activation ratio 336.1 | / | / |

The results of the experiment indicate that human AKR1C3 can activate the in vitro cytotoxicity of compounds H1˜H44 above and compounds with similar structures; in other words, this series of compounds are anticancer gemcitabine prodrugs activated by AKR1C3. Specifically, by comparing the compounds on the left side of the above table with comparative compounds D1 to D10 on the right side, it can be seen that compounds of structural formula (I) where the N atom connected to R₁ is an imine structure (—NH—) have higher in vitro cytotoxicity compared with compounds of structural formula (comparative I) where the N atom connected to R₁ is a tertiary amine group (compared when the numbers of carbon atoms connected to N and the structures are similar): generally, both the values corresponding to IC₅₀ (nM) in the table and the values corresponding to +AST-3021 IC₅₀ (nM) in the table are lower (i.e. compounds of structural formula (I) have stronger cytotoxicity), and compound (I) has a higher AKR1C3 specific activation ratio than compound (comparative I).

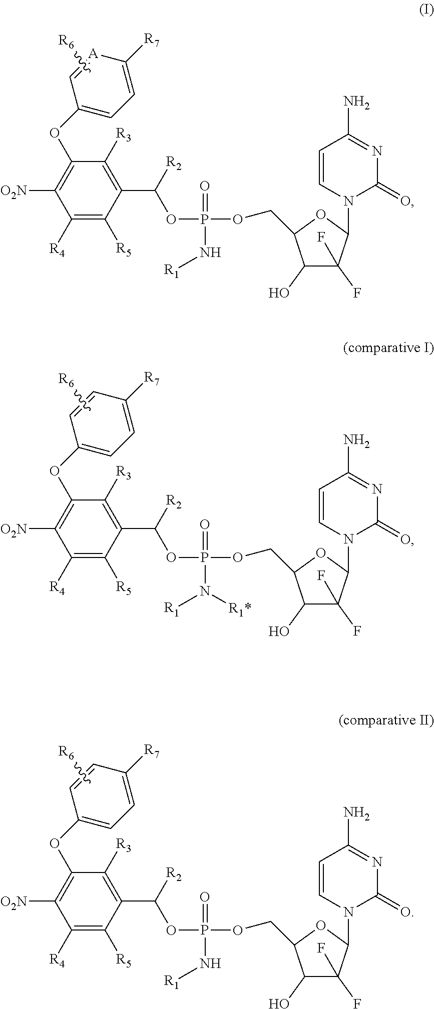

Further, the experiment also confirmed that compounds of structural formula (comparative II) were not activated by AKR1C3 at all, and might not be substrates of AKR1C3.

That is to say, the present invention corrects the partially inaccurate view as indicated in the patent application entitiled NITROBENZYL DERIVATIVES OF ANTI-CANCER AGENTS with Patent Application No. PCT/US2016/025665 and Publication No. WO2016/161342A1, corresponding to Chinese Patent Application No. 201680020013.2 (Publication No. CN108136214A)), that nitrobenzyl derivatives:

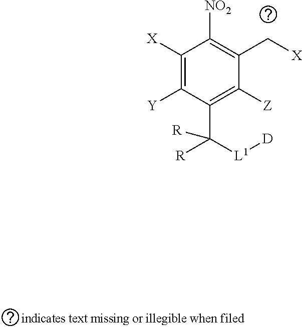

are all AKR1C3 activated anticancer drugs. In other words, the present invention clarifies that for gemcitabine-nitrobenzyl derivatives, compounds of structural formula (comparative II) are not activated by AKR1C3 at all, and finds by comparison of examples that compounds of structural formula (I) have higher cancer cell proliferation inhibition activity than compounds of structural formula (comparative I).

### II. Experiment on AKR1C3 Activation Dependent Inhibitory Activity Against Cell Proliferation

### 2.1 Test of Inhibition by Gemcitabine and Compound H11 on H460 Cancer Cells in the Presence or Absence of the AKR1C3 Inhibitor AST-3021

The experimental scheme was as follows:
1) H460 cell suspension was added to a 96-well plate with 100 μL per well at a cell density of 2000/well.
2) Cells were incubated in an incubator of 37° C., 5% CO₂ overnight.
3) Compound treatment

Combined administration: 24 hours after cell plating, 98 μL of growth medium per well was added. 1 μL of AST-3021 was added to each well (final concentration 3 μM), and the plate was shaken gently to ensure even mixing, and then placed into an incubator; after 2 hours, 1 μL of the test compound at different concentrations was added, and the plate was shaken gently to ensure even mixing, and then placed into an incubator of 37° C., 5% CO₂.

Administration of the drug alone: 24 hours after cell plating, 99 μL of growth medium per well was added. 1 μL of the test compound at different concentrations was added, and the plate was shaken gently to ensure even mixing, and then placed into an incubator of 37° C., 5% CO₂.
4) The cell plate was placed into an incubator for 72 hours.
5) The cell plate to be tested was placed at room temperature to equilibrate for 30 minutes, and 100 μL of medium per well was discarded.
6) 25 μL of CTG reagent per well was add, and the plate was placed in a high-speed shaker to oscillate for 2 minutes, and placed at room temperature in the dark for 30 minutes.
7) The chemiluminescence signal value was read using a multimode microplate reader, with a read time of 1000 ms.
8) IC₅₀ was calculated using GraphPad Prism 5 software, and the IC₅₀ (half inhibitory concentration) of the compound was obtained using the nonlinear fitting formula.

In order to further verify that the compound was activated specifically by human AKR1C3 (aldosterone reductase family 1 member C3), the compound was added following the pretreatment of cells with the specific AKR1C3 enzyme inhibitor AST-3021 (3 μM), and the effect of the presence of AST-3021 on the proliferation of H460 cancer cells was tested

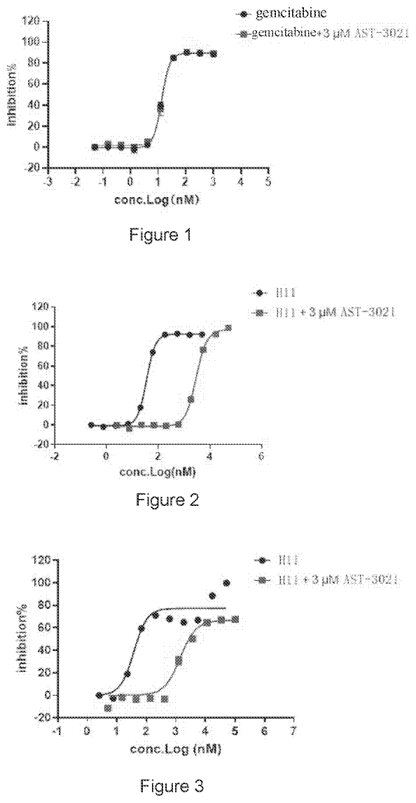

IC₅₀ data of inhibitory effect of the compounds gemcitabine and H11 on H460 cancer cells in the presence or absence of the AKR1C3 inhibitor AST-3021 is shown in Table 2 and Table 3. IC₅₀ curves corresponding to gemcitabine are shown in FIG. 1. IC₅₀ curves of compound H11 corresponding to experiment No. 3 are shown in FIG. 2.

TABLE 2
IC₅₀ data of inhibitory effect of gemcitabine on H460 cancer
cells in the presence or absence of the AKR1C3 inhibitor

| | IC₅₀ (nmol/L) | | Specific |
|---|---|---|---|
| | AST-3021 | AST-3021 | activation |
| Compound | not added | added | ratio |
| gemcitabine | 13.19 | 14.21 | 1.08 |

TABLE 3
IC₅₀ data of inhibitory effect of compound H11 on H460 cancer
cells in the presence or absence of AKR1C3 inhibitor

| | | IC₅₀ (nmol/L) | | Specific |
|---|---|---|---|---|
| Experiment | | AST-3021 | AST-3021 | activation |
| No. | Compound | not added | added | ratio |
| 1 | H11 | 16.25 | 1473.00 | 90.65 |
| 2 | H11 | 30.29 | 2348.00 | 77.52 |
| 3 | H11 | 35.70 | 2942.00 | 80.41 |

The test results show that the IC₅₀ values of gemcitabine with or without the addition of AST-3021 were close, with a specific activation ratio of only 1.08, indicating that the inhibitory activity of gemcitabine on cancer cells is not dependent on the expression of AKR1C3. However, the IC₅₀ values of compound H11 with or without the addition of AST-3021 vary significantly, with specific activation ratios reaching over 75 as shown by all the test results of three different times. This together with the relation between IC₅₀ specific activation ratios of gemcitabine and the addition of AST-3021 demonstrates that compound H11 is an AKR1C3 activation dependent anticancer compound, and its in vitro activity is closely related to the expression of AKR1C3 enzyme.

### 2.2 Test of Inhibition by Compound H11 on HepG2 Cancer Cells in the Presence or Absence of the AKR1C3 Inhibitor AST-3021

HepG2 cells with high expression of human AKR1C3 were selected, and IC₅₀ data of compound H11 on HepG2 cancer cells in the presence or absence of the AKR1C3 inhibitor AST-3021 was tested using the same test method as 2.1, as shown in Table 4. IC₅₀ curves corresponding to experiment No. 5 are shown in FIG. 3.

TABLE 4
IC₅₀ data of inhibitory effect of compound H11 on HepG2 cancer
cells in the presence or absence of AKR1C3 inhibitor

| | | IC₅₀ (nmol/L) | | Specific |
|---|---|---|---|---|
| Experiment | | AST-3021 | AST-3021 | activation |
| No. | Compound | not added | added | ratio |
| 4 | H11 | 42.85 | 3350.00 | 78.18 |
| 5 | H11 | 39.82 | 1582.00 | 39.73 |
| 6 | H11 | 80.89 | 1660.00 | 20.52 |

The test results show that the IC₅₀ values of compound H11 with or without the addition of AST-3021 vary significantly, with specific activation ratios reaching over 20 as shown by all the test results of three different times, demonstrating that compound H11 is an AKR1C3 activation dependent anticancer compound for HepG2 cancer cells, and its in vitro activity is closely related to the expression of AKR1C3 enzyme.

### III. Comparison of Compound H11 and Gemcitabine in the In Vivo Pharmacodynamic Experiment in Pancreatic Cancer PA1222 Model

Description of the experiment: Tumor tissue was collected from tumor bearing mice of HuPrime® pancreatic cancer xenograft model PA1222 (pancreatic cancer PDX model with high expression of AKR1C3), cut into tumor blocks with a diameter of 2-3 mm and inoculated subcutaneously at the right anterior scapula of BALB/c nude mice. When the average volume of tumor was about 102 mm³, mice were randomly divided into groups according to the size of the tumor. Table 5 shows the design of the experiment on antitumor effect of test drugs in HuPrime® pancreatic cancer PA1222 tumor model.

TABLE 5
Design of the experiment on antitumor effect of test drugs in
HuPrime ® pancreatic cancer PA1222 tumor model

| | Number | | | | |
|---|---|---|---|---|---|
| | of | | Dosage | Administration | Administration |
| Group | animals | Administration | (mg/kg) | method | cycle |
| 1 | 5 | 10% anhydrous ethanol + | — | tail vein | Q2D × 3 weeks |
| | | 10% polyoxyl 35 castor oil + | | injection (i.v.) | |
| | | 80% glucose injection D5W | | | |
| | | (pH 7.7-8.0) | | | |
| 2 | 5 | gemcitabine | 80 | intraperitoneal | Q3D × 4 |
| | | | | injection (i.p.) | |
| 3 | 5 | H11 | 240 | intragastric | QD × 21 |
| | | | | administration | |
| | | | | (p.o.) | |
| 4 | 5 | H11 | 160 | intraperitoneal | QD × 5, 2 days |
| | | | | injection (i.p.) | off, 2 weeks |
| | | | | | off; QD × 5 |
| 5 | 5 | H11 | 160 | tail vein | Q2D × 3 weeks |
| | | | | injection (i.v.) | |

Note:
1. The dosage volume was 10 μL/g.
2. Q3D × 4 means performing administration once every three days for four consecutive administrations; Q2D × 3 means performing administration once every two days for three consecutive weeks; QD × 21 means performing administration once a day for 21 consecutive days; QD × 5, 2 days off, 2 weeks off; QD × 5 means performing administration once a day for 5 consecutive days, stopping administration for 2 days, and for 2 weeks, and then performing administration continuously for 5 days, once a day.

Group 2, Group 3, Group 4, and Group 5 were ended on the 32nd day after the first administration, i.e. Day 32. Tumors were collected, weighed, photographed, and quick-frozen. On Day 57, Group 1 was ended, and tumors were collected, weighed, photographed, and quick-frozen. Efficacy evaluation was performed according to the relative tumor growth inhibition (TGI %), and safety evaluation was performed according to changes in animal weight and mortality.

Standard for efficacy evaluation and calculation methods were as follows:

The relative tumor proliferation rate, T/C %, is the percent ratio of relative tumor volume or weight between the treatment group and the control group at a certain time point. The calculation formula is as follows: T/C %=T_{RTV}/C_{RTV}×100% (T_{RTV}: average RTV of the treatment group; C_{RTV}: average RTV of the solvent control group; RTV=Vₜ/V₀, V₀ is the tumor volume of the animal at the time of grouping, and Vₜ is the tumor volume of the animal after treatment);

The relative tumor growth inhibition, TGI (%), is calculated as follows: TGI %=(1−T/C)×100% (T and C are the average relative tumor volume (RTV) of the treatment group and the control group at a specific time point, respectively).

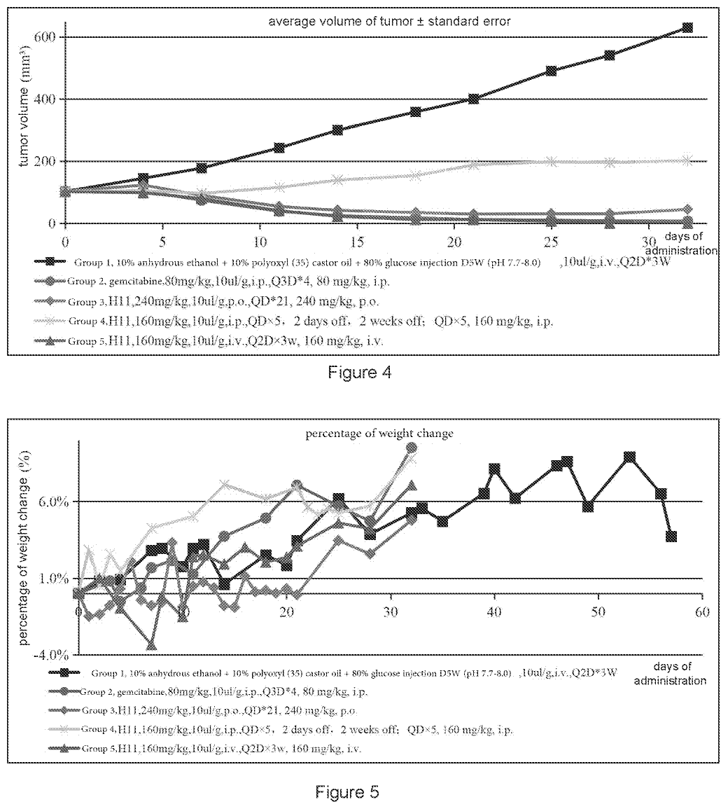

The efficacy of each group in the pancreatic cancer PA1222 model was analyzed on the 32nd day after the first administration, and the results are shown in Table 6. The average volumes of tumors on different days were recorded and averaged as shown in Table 7. The average tumor volumes in the first 32 days after the start of administration were plotted into curves, as shown in FIG. 4.

TABLE 6
Pharmacodynamic analysis of each group in the HuPrime ® pancreatic
cancer PA1222 model

| | The 32nd day after the first administration (i.e. Day 32, Oct. 4, 2021) | | | | |
|---|---|---|---|---|---|
| Group | | Relative | | | P-value |
| of the | Tumor volume | tumor volume | TGI | T/C | (vs. the |
| experiment | (x̅ ± S) | (x̅ ± S) | (%) | (%) | control group) |
| Group 1 | 629.85 ± 136.87 | 6.21 ± 1.36 | — | — | — |
| Group 1 | 1247.07 ± 307.89 | 12.15 ± 2.89 | — | — | — |
| | (Day 56) | | | | |
| Group 2 | 6.79 ± 4.33 | 0.06 ± 0.04 | 99.03% | 0.97% | <0.001 |
| Group 3 | 44.61 ± 22.14 | 0.47 ± 0.27 | 92.35% | 7.65% | <0.001 |
| Group 4 | 202.58 ± 45.40 | 2.01 ± 0.41 | 67.62% | 32.38% | <0.05 |
| Group 5 | 0.00 ± 0.00 | 0.00 ± 0.00 | 100.00% | 0.00% | <0.001 |

Note:
1. The data is represented as “mean ± standard error”;
2. T/C % = T_{RTV}/C_{RTV} × 100%; TGI % = (1 − T/C) × 100%.

TABLE 7
Tumor volume change of mice of each group in the HuPrime ®
pancreatic cancer PA1222 model with treatment time
Tumor volume (mm³) (x̅ ± S)

| Time | Group 1 | Group 2 | Group 3 | Group 4 | Group 5 |
|---|---|---|---|---|---|
| the 0th day of | 102.69 ± | 102.95 ± | 102.83 ± | 101.91 ± | 102.11 ± |
| administration | 6.88 | 6.86 | 6.64 | 5.96 | 6.00 |
| the 4th day of | 144.96 ± | 104.07 ± | 123.16 ± | 105.94 ± | 98.46 ± |
| administration | 13.68 | 9.22 | 3.48 | 8.67 | 3.72 |
| the 7th day of | 177.74 ± | 74.43 ± | 90.07 ± | 96.63 ± | 79.97 ± |
| administration | 24.98 | 5.22 | 4.65 | 11.34 | 3.69 |
| the 11th day of | 243.12 ± | 38.92 ± | 54.28 ± | 116.07 ± | 40.67 ± |
| administration | 34.25 | 4.90 | 5.75 | 22.87 | 3.20 |
| the 14th day of | 299.90 ± | 24.66 ± | 41.73 ± | 139.61 ± | 22.37 ± |
| administration | 51.57 | 3.94 | 7.84 | 22.61 | 2.70 |
| the 18th day of | 358.17 ± | 17.66 ± | 34.60 ± | 153.86 ± | 11.52 ± |
| administration | 54.38 | 4.82 | 7.08 | 27.60 | 4.90 |
| the 21st day of | 399.99 ± | 12.13 ± | 29.52 ± | 188.69 ± | 11.56 ± |
| administration | 56.79 | 3.15 | 12.59 | 33.45 | 3.08 |
| the 25th day of | 490.19 ± | 10.23 ± | 30.71 ± | 198.68 ± | 6.73 ± |
| administration | 78.73 | 3.05 | 15.00 | 40.97 | 2.81 |
| the 28th day of | 540.46 ± | 7.97 ± | 30.59 ± | 195.90 ± | 0.00 ± |
| administration | 97.48 | 3.70 | 12.12 | 41.71 | 0.00 |
| the 32nd day of | 629.85 ± | 6.79 ± | 44.61 ± | 202.58 ± | 0.00 ± |
| administration | 136.87 | 4.33 | 22.14 | 45.40 | 0.00 |
| the 35th day of | 669.64 ± | / | / | / | / |
| administration | 145.73 | | | | |
| the 39th day of | 799.46 ± | / | / | / | / |
| administration | 184.86 | | | | |
| the 42nd day of | 912.51 ± | / | / | / | / |
| administration | 196.70 | | | | |
| the 46th day of | 1004.17 ± | / | / | / | / |
| administration | 295.28 | | | | |
| the 49th day of | 1064.65 ± | / | / | / | / |
| administration | 273.52 | | | | |
| the 53rd day of | 1154.21 ± | / | / | / | / |
| administration | 310.62 | | | | |
| the 56th day of | 1247.07 ± | / | / | / | / |
| administration | 307.89 | | | | |

In order to verify the effect of test drugs on the weight change of experimental animals, according to the above experimental method, the weight change of experimental animals were recorded simultaneously at different time points, as shown in Table 8. The weight growth rate was calculated and plotted as curves, as shown in FIG. 5.

TABLE 8
Average weight (g) of mice on different days

| Day | 0 | 1 | 2 | 3 | 4 | 5 | 6 | 7 | 8 | 9 |
|---|---|---|---|---|---|---|---|---|---|---|
| Group 1 | 22.2 | / | 22.4 | / | 22.4 | / | / | 22.9 | 22.9 | / |
| Group 2 | 21.1 | / | / | 21.2 | 21.0 | / | 21.2 | 21.4 | / | 21.5 |
| Group 3 | 21.8 | 21.5 | 21.5 | 21.6 | 21.9 | 22.3 | 21.7 | 21.6 | 21.7 | 22.5 |
| Group 4 | 21.4 | 22.0 | 21.5 | 22.0 | 21.7 | / | / | 22.3 | / | / |
| Group 5 | 21.6 | / | 21.8 | / | 21.4 | / | / | 20.9 | 21.6 | / |

| Day | 10 | 11 | 12 | 13 | 14 | 15 | 16 | 17 | 18 | 19 |
|---|---|---|---|---|---|---|---|---|---|---|
| Group 1 | 22.6 | 22.9 | 23.0 | / | 22.4 | / | / | / | 22.8 | / |
| Group 2 | / | 21.3 | / | / | 21.9 | / | / | / | 22.1 | / |
| Group 3 | 21.6 | 21.9 | 22.0 | 21.9 | 21.6 | 21.6 | 22.1 | 21.8 | 21.9 | 21.8 |
| Group 4 | / | 22.5 | / | / | 22.9 | / | / | / | 22.7 | / |
| Group 5 | 21.3 | 22.1 | 22.2 | / | 22.0 | / | 22.3 | / | 22.1 | / |

| Day | 20 | 21 | 22 | 23 | 24 | 25 | 26 | 28 | 32 | 33 |
|---|---|---|---|---|---|---|---|---|---|---|
| Group 1 | 22.6 | 23.0 | / | / | / | 23.6 | / | 23.1 | 23.4 | 23.5 |
| Group 2 | / | 22.6 | / | / | / | 22.3 | / | 22.1 | 23.1 | / |
| Group 3 | 21.9 | 21.8 | / | / | / | 22.6 | / | 22.4 | 22.9 | / |
| Group 4 | / | 22.9 | 22.6 | 22.5 | 22.6 | 22.5 | / | 22.6 | 23.3 | / |
| Group 5 | 22.1 | 22.3 | / | / | / | 22.6 | / | 22.5 | 23.2 | / |

| Day | 35 | 39 | 40 | 42 | 46 | 47 | 49 | 53 | 56 | 57 |
|---|---|---|---|---|---|---|---|---|---|---|
| Group 1 | 23.3 | 23.7 | 24.1 | 23.6 | 24.1 | 24.2 | 23.5 | 24.2 | 23.7 | 22.9 |
| Group 2 | / | / | / | / | / | / | / | / | / | / |
| Group 3 | / | / | / | / | / | / | / | / | / | / |
| Group 4 | / | / | / | / | / | / | / | / | / | / |
| Group 5 | / | / | / | / | / | / | / | / | / | / |

The above experimental data indicates that:

Test drugs gemcitabine (80 mg/kg, Group 2), H11 240 mg/kg (Group 3, QD×21) and H11 160 mg/kg, i.v. (Group 5, Q2D×3 weeks) treatment groups showed extremely significant antitumor effect on the 32nd day after the first administration (Day 32), with relative tumor growth inhibition (TGI (%)) of 99.03%, 92.35%, and 100%, and complete tumor inhibition rates of 60%, 20%, and 100%, respectively. H11 160 mg/kg, i.p. (Group 4, QD×5, 2 days off, 2 weeks off; QD×5) treatment group showed significant antitumor effect on the 32nd day after the first administration (Day 32), with a relative tumor growth inhibition TGI (%) of 67.62%. Meanwhile, the antitumor effect of test drug H11 160 mg/kg, i.v. (Group 5, Q2D×3 weeks) was better than that of H11 160 mg/kg, i.p. (Group 4, QD×5, 2 days off, 2 weeks off; QD×5), and there was a significant difference in statistics between the two groups (Group 5 and Group 4) (P<0.001). The antitumor effect of test drug H11 160 mg/kg, i.v. (Group 5, Q2D×3 weeks) was better than that of H11 240 mg/kg, p.o. (Group 3, QD×21), and there was a significant difference in statistical comparison between the two groups (Group 5 and Group 3) (P<0.05). The antitumor effect of test drug H11 160 mg/kg, i.v. (Group 5, Q2D×3 weeks), H11 240 mg/kg, p.o. (Group 3, QD×21) was not significantly different from that of the test drug gemcitabine (80 mg/kg, Group 2) treatment group, and there was no statistically significant difference between any two of the groups (Group 5 vs Group 2 and Group 3 vs Group 2) (P-values greater than 0.05).

The mice in the test drug gemcitabine 80 mg/kg treatment group, H11 240 mg/kg (Group 3, QD×21) treatment group, H11 160 mg/kg, i.p. (Group 4, QD×5, 2 days off, 2 weeks off; QD×5) treatment group, and test drug H11 160 mg/kg, i.v. (Group 5, Q2D×3 weeks) treatment group and the control group did not show any significant weight loss, and tolerated well during the treatment period.

### IV. Experiment on Evaluation of Metabolic Stability

### 4.1 Study on Metabolic Stability of Compound H11 and the Positive Control Verapamil in Liver Microsomes

### Experimental Scheme:

### (I) Solution Preparation

Main solutions were prepared according to Table 9 below:

TABLE 9
Preparation of main solutions

| | Material | | | | Final | |
|---|---|---|---|---|---|---|
| Reagent | concentration | | Volume | | concentration | |
| phosphate buffer | 100 | mM | 216.25 | μL | 100 | mM |
| microsome | 20 | mg/mL | 6.25 | μL | 0.5 | mg/mL |

(II) Two Separate Experiments were Performed
a) With cofactor (NADPH): 25 μL of 10 mM NADPH was added to the culture broth. The final concentration of liver microsomes was 0.5 mg/mL, and the final concentration of NADPH was 1 mM.
b) Without cofactor (NADPH): 25 μL of 100 mM phosphate buffer was added. The final concentration of liver microsomes was 0.5 mg/mL. The mixture was preheated at 37° C. for 10 minutes.
   (III) 25 μL of 100 mM control compound or test compound solution was added to start the reaction. This study used verapamil as a positive control. The final concentrations of the test compound and control compound were both 1 μM. The culture broth was cultured in water at 37° C.
   (IV) Aliquots of 30 μL were collected from the reaction solution at 0.5, 5, 15, 30, and 60 minutes, respectively. 5 volumes of cold acetonitrile and IS (internal standard, containing 100 nM of alprazolam, 200 nM of caffeine, and 100 nM of tolylbutanamide) were added to stop the reaction. Samples were centrifuged at a speed of 3,220 g for 40 minutes. 100 μL of supernatant was taken and mixed with 100 μL of ultrapure water, equally divided and analyzed with LC-MS/MS.

### (V) Data Analysis

All calculations were conducted using Microsoft Excel. The peak area was determined from the extracted ion chromatogram. The slope value k was determined by linear regression of the natural logarithm of the remaining percentage of the parent drug vs. incubation time curve.
(1) The in vitro half-life (in vitro t_{1/2}) was determined by the slope value: in vitro t_{1/2}=−0.693/k.
(2) In vitro t_{1/2} (min) was converted into in vitro intrinsic clearance (in vitro CLᵢₙₜ, μL/min/1×10⁶ cells), using the following formula (average of repeated measurements): CLᵢₙₜ=−kV/N, wherein V=culture volume (0.2 mL) and N=number of liver cells per well (0.1×10⁶ cells).
(3) Scale-up CLᵢₙₜ (mL/min/kg), Predicted Hepatic CL_{H} (mL/min/kg), and Hepatic Extraction Ratio (ER) were calculated using the following formulas:

$$\\ Scale ‐ up {CL}_{int} = ( 0.693 / {T}_{1 / 2} ) \times ( 1 / ( liver cell concentration ( 0.5 \times {10}^{{}_{} 6} cells / mL ) ) ) \times Scaling Factor ( Table 10 ) ;$$

$$\\ Predicted Hepatic {CL}_{H} = ( QH \times Scale ‐ up {CL}_{int} \times fub ) / ( QH + Scale ‐ up {CL}_{int} \times fub ) ;$$

$$\\ ER = Predicted Hepatic {CL}_{H} / QH ;$$

wherein, QH is the hepatic blood flow (mL/min/kg) in Table 10, and fub is the fraction of drug unbound in plasma, and assumed as 1.

TABLE 10
Scaling Factors for Intrinsic Clearance Prediction
in human, monkey, dog, rat, and mouse hepatocytes

| | Liver | | | |
|---|---|---|---|---|
| | microsome | | | |
| | protein | Liver weight per | | Hepatic Blood |
| | per gram of | kilogram of | Scaling | Flow |
| Species | liver | body weight | Factor | (mL/min/kg) |
| human | 40 | 25.7 | 1028 | 21 |
| monkey | 40 | 30 | 1200 | 44 |
| dog | 55 | 32 | 1760 | 31 |
| rat | 61 | 40 | 2440 | 68 |
| mouse | 47 | 88 | 4136 | 90 |

Note:
Scaling Factor = (liver microsome protein per gram of liver) × (liver weight per kilogram of body weight).

### Results of the Experiment:

The above experimental scheme was used to detect and calculate the metabolic stability data of compound H11 and the positive control verapamil in human, monkey, dog, rat, and mouse liver microsomes. Table 11 shows the change of the remaining percentage of compound H11 and verapamil in human, monkey, dog, rat, and mouse liver microsomes over time. Table 12 shows the metabolic stability data of compound H11 and verapamil in human, monkey, dog, rat, and mouse liver microsomes.

TABLE 11
Change of the remaining percentage of compound H11 and verapamil
in human, monkey, dog, rat, and mouse liver microsomes over time

| | | | Remaining percentage (%) | | | | |
|---|---|---|---|---|---|---|---|
| | | Cofactor | 0.5 | 5 | 15 | 30 | 60 |
| Compound | Species | added or not | minutes | minutes | minutes | minutes | minutes |
| verapamil | human | NADPH added | 100.00 | 40.59 | 9.10 | 2.75 | BLOD |
| | | NADPH not added | 100.00 | — | — | — | 104.85 |
| | monkey | NADPH added | 100.00 | 5.34 | BLOD | BLOD | BLOD |
| | | NADPH not added | 100.00 | — | — | — | 104.77 |
| | dog | NADPH added | 100.00 | 31.34 | 5.52 | 0.66 | BLOD |
| | | NADPH not added | 100.00 | — | — | — | 93.24 |
| | rat | NADPH added | 100.00 | 27.74 | 2.94 | BLOD | BLOD |
| | | NADPH not added | 100.00 | — | — | — | 103.79 |
| | mouse | NADPH added | 100.00 | 10.79 | BLOD | BLOD | BLOD |
| | | NADPH not added | 100.00 | — | — | — | 98.85 |
| H11 | human | NADPH added | 100.00 | 86.00 | 63.82 | 43.87 | 26.14 |
| | | NADPH not added | 100.00 | — | — | — | 100.86 |
| | monkey | NADPH added | 100.00 | 46.01 | 13.27 | 0.59 | 0.09 |
| | | NADPH not added | 100.00 | — | — | — | 105.87 |
| | dog | NADPH added | 100.00 | 90.20 | 71.99 | 52.53 | 32.12 |
| | | NADPH not added | 100.00 | — | — | — | 92.14 |
| | rat | NADPH added | 100.00 | 85.67 | 56.17 | 29.55 | 13.20 |
| | | NADPH not added | 100.00 | — | — | — | 103.20 |
| | mouse | NADPH added | 100.00 | 85.55 | 55.03 | 36.05 | 16.85 |
| | | NADPH not added | 100.00 | — | — | — | 93.32 |

Note:
BLOD indicates that the result is below the detection limit.

TABLE 12
Metabolic stability data of compound H11 and verapamil
in human, monkey, dog, rat, and mouse liver microsomes

| | | In vitro | In vitro | | | Hepatic |
|---|---|---|---|---|---|---|
| | | half-life | intrinsic | Scale-up | Predicted | Extraction |
| | | t_{1/2} | clearance CLᵢₙₜ | CLᵢₙₜ | Hepatic CL_{H} | Ratio |
| Compound | Species | (min) | (μL/min/mg) | (mL/min/kg) | (mL/min/kg) | (ER) |
| verapamil | human | 3.46 | 400.78 | 412.00 | 19.98 | 0.95 |
| | monkey | 1.06 | 1304.60 | 1565.52 | 42.80 | 0.97 |
| | dog | 2.69 | 515.70 | 907.63 | 29.98 | 0.97 |
| | rat | 2.43 | 569.96 | 1390.69 | 64.83 | 0.95 |
| | mouse | 1.40 | 989.53 | 4092.70 | 88.06 | 0.98 |
| H11 | human | 31.01 | 44.71 | 45.97 | 14.41 | 0.69 |
| | monkey | 5.06 | 274.95 | 329.94 | 38.81 | 0.88 |
| | dog | 36.33 | 38.16 | 67.17 | 21.21 | 0.68 |
| | rat | 20.11 | 68.99 | 168.33 | 48.43 | 0.71 |
| | mouse | 23.30 | 59.52 | 246.18 | 65.90 | 0.73 |

### Conclusion of the Experiment:

During the incubation process, the remaining percentage of the positive control verapamil significantly decreased with the prolongation of incubation time (Table 12), which is consistent with the characteristics of this positive drug. Therefore, the activity of liver microsomes in this experiment was normal, and the experimental results of test drug H11 are reliable.

The results show that the intrinsic clearances of H11 calculated after incubation with human, monkey, dog, rat, and mouse liver microsomes were 44.71 μL/min/10⁶, 274.95 μL/min/10⁶, 38.16 μL/min/10⁶, 68.99 μL/min/10⁶, and 59.52 μL/min/10⁶ cells, respectively. H11 has a high clearance and poor stability in monkey liver microsomes; and medium clearances and certain stability in human, dog, rat, and mouse liver microsomes.

### 4.2 Study on Plasma Stability of Compound H11

### Experimental Scheme:

### (I) Solution Preparation

1 mM working solution of the test compound was prepared in DMSO. 1 mM working solution of propantheline was prepared in acetonitrile. 1 mM working solution of lovastatin (mevinolin) was prepared in DMSO. Propantheline was used as a positive control for human, monkey, dog, and mouse plasma stability tests. Lovastatin was a positive control for rat plasma stability test.

### (II) Experimental Process

A. 398 μL of plasma per well was added to the culture plate, and the plate was preheated at 37° C. for 15 minutes.
B. After preincubation, 2 μL of working solution (test solution or control solution) was added to the 398 μL plasma mentioned above, resulting in a final concentration of 5 μM. The final concentration of organic solvents was 0.5%. The test was conducted in duplicate.
C. The reaction sample was incubated at 37° C.
D. 50 μL was extracted from the reaction sample at 0, 15, 30, 60, and 120 minutes, respectively. 450 μL of cold acetonitrile with internal standard was added to stop the reaction.
E. All samples were vortexed for 10 minutes, followed by centrifugation at 3220 g for 30 minutes to precipitate the protein. 100 μL of supernatant was taken and transferred to a new plate. The supernatant was diluted with ultrapure water according to the LC-MS signal response and peak shape.

### (III) Data Analysis

Samples were analyzed with LC-MS/MS. All calculations were conducted using Microsoft Excel. The peak area ratio was determined from the extracted ion chromatogram.
(1) The percentage of the remaining compound at each time point was calculated with the following formula:

$$\\ remaining percentage t min ( \% ) = peak area ratio t min / peak area ratio 0 min \times 100 ,$$

wherein the peak area ratio t min is the peak area ratio of the control compound to the test compound at t min, and the peak area ratio 0 min is the peak area ratio of the control compound to the test compound at zero time point.
(2) The in vitro half-life (in vitro t_{1/2}) was determined by the slope value:

$$\\ {t}_{1 / 2} = - 0.693 / k ,$$

wherein the slope value k was determined by linear regression of the natural logarithm of the remaining percentage of the parent drug vs. incubation time curve.

### Results of the Experiment:

The above experimental scheme was used to detect and calculate the metabolic stability data of compound H11 and the positive controls propantheline and lovastatin (mevinolin) in human, monkey, dog, rat, and mouse plasma, respectively. Table 13 shows the change of the remaining percentage of compound H11 and the positive controls propantheline and lovastatin in human, monkey, dog, rat, and mouse plasma over time.

### Table 13: Change of the Remaining Percentage of Compound H11 and Propantheline and Lovastatin in Plasma Over Time

TABLE 13
Change of the remaining percentage of compound H11 and propantheline and lovastatin
in plasma over time

| | | Remaining percentage (%) | | | | | t_{1/2} |
|---|---|---|---|---|---|---|---|
| Compound | Species | 0 min | 15 min | 30 min | 60 min | 120 min | (min) |
| propantheline | human | 100.00 | 81.24 | 59.99 | 32.76 | 7.75 | 36.64 |
| | monkey | 100.00 | 90.53 | 77.57 | 70.39 | 49.22 | 121.02 |
| | dog | 100.00 | 101.04 | 87.98 | 85.64 | 66.64 | 202.71 |
| lovastatin | rat | 100.00 | 5.27 | 0.52 | 0.45 | 0.34 | 3.53 |
| (mevinolin) | | | | | | | |
| propantheline | mouse | 100.00 | 69.89 | 43.11 | 15.28 | 2.03 | 21.83 |
| H11 | human | 100.00 | 95.74 | 94.17 | 102.77 | 99.46 | ∞ |
| | monkey | 100.00 | 100.61 | 92.99 | 103.66 | 98.48 | ∞ |
| | dog | 100.00 | 104.14 | 103.37 | 102.42 | 103.04 | ∞ |
| | rat | 100.00 | 108.59 | 105.39 | 100.74 | 109.53 | ∞ |
| | mouse | 100.00 | 102.19 | 96.83 | 104.70 | 97.53 | ∞ |

### Conclusion of the Experiment:

The experimental results of the positive controls propantheline and lovastatin are consistent with the characteristics of these positive drugs; therefore the activity of different plasma used in this experiment was normal, and the experimental results of test drug H11 are reliable.

Research data shows that H11 is stable and has no significant metabolism when incubated at 37° C. in mouse, rat, dog, monkey, and human plasma for at least 2 hours.

### V. Maximal Tolerance Dose (MTD) Test

### Experimental Process:

According to the research scheme in Table 14, mice were grouped and administered at the concentrations and dosage levels shown in Table 14. On the 1st to 5th day and the 14th to 19th day, intraperitoneal injection of a solvent control or compound H11 at different dosage levels was performed, with a dose volume of 10 mL/kg. Animals in Groups 1, 3, and 4 were put to death and autopsied on the 22nd day, and animals in Group 2 were put to death and autopsied on the 16th day.

TABLE 14
Research scheme

| | | Dosage level of | Concen- | Number |
|---|---|---|---|---|
| | Treatment | administration | tration | of mice |
| Group | drug | (mg/kg/day) | (mg/mL) | (male/female) |
| 1 | blank vehicle^{a} | / | / | 2/2 |
| 2 | H11 | 80 | 8 | 3/3 |
| 3 | H11 | 160 | 16 | 3/3 |
| 4 | H11 | 320 | 32 | 3/3 |

Note:
^{a}conventional 10% polyoxyl (35) castor oil and 10% ethanol dissolved in 5% glucose injection.

The evaluated parameters included cage-side observation, detailed clinical observation, and post-administration observation (approximately 1-2 hours after dosing), body weight and weight change, food intake, and gross pathology.

### Experimental Results:

### (1) Mortality of Animals

During the experiment, all experimental animals survived until the planned autopsy.

### (2) Clinical Symptoms

No abnormal compound-related clinical manifestation was observed.

### (3) The Impact on Animal Weight and Food Intake

No abnormal compound-related change in body weight or food intake was observed.

### (4) Pathological Examination Results

Results of the compound-related pathological examination mainly include enlargement of the spleen visible at ≥160 mg/kg, and a change to white in the renal capsule visible in the 320 mg/kg dose group, and no other abnormalities.

### Experimental Conclusion:

Under the conditions of this experiment, mice were administered with compound H11 once a day for 5 consecutive days through two rounds of intraperitoneal injection. The safety to animals was good, and no abnormalities were observed except for spleen enlargement and changes in the renal capsule of the 320 mg/kg dose group as observed through autopsy. Therefore, the maximal tolerance dose under the conditions of this experiment can reach 320 mg/kg (intraperitoneal injection once a day for 5 consecutive days). However, the MTD of gemcitabine reported by literature is 120 mg/kg (intraperitoneal injection, once every 3 days, for four consecutive doses) (Cancer Chemotherapy and Pharmacology 38 (4): 335-42). It is evident that as a prodrug of gemcitabine, H11 has a greater tolerance dose and is safer than gemcitabine.

### VI. Synthesis of Compounds

Description of reagents and instruments used is as follows:

THF stands for tetrahydrofuran; DCM stands for dichloromethane; EA or EtOAC stands for ethyl acetate; TEA stands for triethylamine; MTBE stands for methyl tert-butyl ether; DMAP stands for 4-dimethylaminopyridine; DBAD stands for di-tert-butyl azodicarboxylate; TFA stands for trifluoroacetic acid; EtOH stands for ethanol; t-BuOH stands for tert-butanol; DMF stands for dimethylformamide; PE stands for petroleum ether; TBAF stands for tetrabutylammonium fluoride; DIPEA stands for N,N-diisopropylethylamine; HPLC stands for high performance liquid chromatography; and LCMS stands for liquid chromatography mass spectrometry.

In the synthesis process, all the chemical reagents and drugs whose sources were not indicated were analytically pure or chemically pure, and purchased from commercial reagent companies. Other English abbreviations mentioned herein are subject to the interpretation in the field of organic chemistry.

### Synthesis of Compound H11

Under N₂ protection, H11-A1 (6.0 g, 12.9 mmol) and POCl₃ (1.2 mL, 12.9 mmol) were added to dry DCM (60 mL), and a DCM (10 mL) solution of TEA (2.2 mL, 15.5 mmol) was dropwise added at −10° C. and stirred. H11-A1 was purchased or synthesized with reference to other research literature or patents (Patent Application No. PCT/US2016/025665 (Publication No. WO2016/161342A1), which corresponds to Chinese Patent Application No. 201680020013.2 (Publication No. CN108136214A)).

After a period of time, the reaction was complete. A DCM solution of H11-A2 (4.6 g, 12.9 mmol, synthesized according to literature or purchased) and TEA (2.2 mL, 15.5 mmol) was added dropwise, and the mixture stirred.

A tetrahydrofuran solution of isopropylamine (2 M, 13.2 mL, 25.9 mmol) was added dropwise to the system, then TEA (5.4 mL, 10.1 mmol) was added, and the mixture was stirred at room temperature. Saturated NH₄Cl solution was added dropwise, and the mixture was filtered, the filtrate separated, and the aqueous phase extracted with DCM. After washing with brine, the organic phase was dried with Na₂SO₄ and concentrated, followed by reversed-phase flash column chromatography to yield H11-D (2.0 g (above 85% content) and 2.0 g (50% content), with an overall yield of 25.2%), which was a light yellow solid. MS: Calculated 923.8, found 924.2 ([M+H]⁺).

Under N₂ protection, H11-D (1.5 g, 1.62 mmol) was added to DCM (15 mL) and TFA (18.7 g, 165.2 mmol) was added. After the mixture was stirred at room temperature, the reaction was monitored till completion. The mixture was adjusted to pH=8 with saturated NaHCO₃ aqueous solution and extracted with DCM. After washing with brine, the organic phase was dried with Na₂SO₄ and concentrated, followed by neutral preparative high performance liquid chromatography (using a mobile phase adjusted to neutral with acid or base) to isolate H11 (550 mg, yield 47.0%), which was a white solid.

¹H-NMR (400 MHz, CD₃OD) δ 8.04 (dd, J=8.4, 3.6 Hz, 1H), 7.60-7.48 (m, 4H), 7.39 (d, J=8.8 Hz, 1H), 7.31 (s, 1H), 7.18 (t, J=8.8 Hz, 2H), 6.94-6.91 (m, 2H), 6.26-6.13 (m, 1H), 5.84 (dd, J=7.6, 4.4 Hz, 1H), 5.12-5.09 (m, 2H), 4.34-4.17 (m, 3H), 4.04-4.01 (m, 1H), 3.37-3.31 (m, 1H), 1.15-1.12 (m, 6H). MS: Calculated MS, 723.2, found, 724.0 ([M+H]⁺).

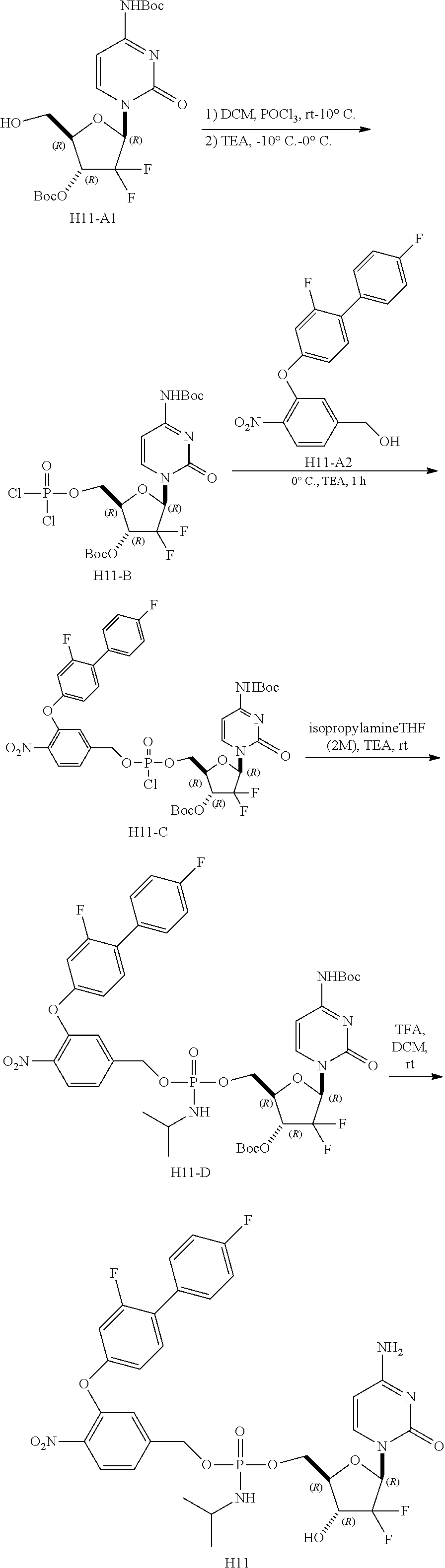

### Synthesis of Other Compounds

The method was similar to the synthesis of compound H11, except that isopropylamine was changed to an organic amine or alcohol with other structures, such as methylamine, ethylamine, n-propylamine, 2-bromoethylamine, isobutylamine, 2-methyl-2-propylamine, 2,2-dimethyl-1-propylamine, benzylamine, cyclohexylamine, cyclopentamine, cyclobutylamine, cyclopropylamine, and 2,2,2-trifluoroethylamine; and Hit-A2 was changed to other corresponding materials with structural formula as follows:

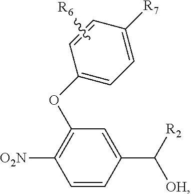

wherein, substituents R₂, R₆, and R₇ are defined as above;

With reference to the synthesis steps of compound H11 mentioned above, other similar compounds could be obtained. The spectral data of other compounds and H11 is listed below:

### Compound H1

The product (9.3 mg, 6.0%) was isolated with neutral preparative high performance liquid chromatography as a white solid.

¹H-NMR (400 MHz, CD₃OD) δ 8.06-8.03 (m, 1H), 7.60-7.40 (m, 5H), 7.32 (s, 1H), 7.18 (t, J=8.8 Hz, 2H), 6.95-6.91 (m, 2H), 6.23-6.18 (m, 2H), 5.86-5.84 (m, 1H), 5.11 (d, J=8.4 Hz, 3H), 4.35-4.16 (m, 3H), 4.04-4.02 (m, 1H), 2.57-2.54 (m, 3H). MS: Calculated 695.1, found 696.2 ([M+H]⁺).

### Compound H2

The product (27.6 mg, 11.7%) was isolated with neutral preparative high performance liquid chromatography as a white solid.

¹H-NMR (400 MHz, CD₃OD) δ 8.06-8.03 (m, 1H), 7.55-7.49 (m, 4H), 7.42-7.39 (m, 1H), 7.31 (s, 1H), 7.21-7.15 (m, 2H), 6.95-6.91 (m, 2H), 6.23-6.17 (m, 1H), 5.85-5.83 (m, 1H), 5.12-5.10 (m, 2H), 4.34-4.16 (m, 3H), 4.04-4.02 (m, 1H), 2.97-2.89 (m, 2H), 1.13-1.08 (m, 3H). MS: Calculated 709.5, found 710.2 ([M+H]⁺).

### Compound H3

The product (71 mg, 29.2%) was isolated with neutral preparative high performance liquid chromatography as a white solid.

¹H-NMR (400 MHz, CD₃OD) δ 8.06-8.03 (m, 1H), 7.56-7.41 (m, 5H), 7.31 (s, 1H), 7.18 (t, J=8.8 Hz, 2H), 6.94-6.91 (m, 2H), 6.23-6.17 (m, 2H), 5.85-5.82 (m, 2H), 5.12-5.10 (m, 2H), 4.34-4.21 (m, 4H), 4.03-4.02 (m, 1H), 2.87-2.81 (m, 2H), 1.51-1.45 (m, 2H), 0.90-0.86 (m, 3H). MS: Calculated 723.2, found 724.0 ([M+H]⁺).

### Compound H4

The product (53.2 mg, 16.7%) was isolated with neutral preparative high performance liquid chromatography as a white solid.

¹H-NMR (400 MHz, CD₃OD) δ 8.04 (dd, J=8.4, 3.2 Hz, 1H), 7.61-7.46 (m, 4H), 7.42-7.40 (m, 1H), 7.32 (s, 1H), 7.20-7.16 (m, 2H), 6.94-6.91 (m, 2H), 6.24-6.20 (m, 1H), 5.87 (d, J=7.6 Hz, 1H), 5.14 (d, J=8.4 Hz, 2H), 4.38-4.18 (m, 3H), 4.06-4.03 (m, 1H), 3.42-3.38 (m, 2H), 3.28-3.25 (m, 2H). MS: Calculated 787.1, found 788.0 and 790.0 ([M+H]⁺).

### Compound H5

The product (26.3 mg, 11.1%) was isolated with neutral preparative high performance liquid chromatography as a white solid.

¹H-NMR (400 MHz, CD₃OD) δ 8.07 (dd, J=8.4, 4.0 Hz, 1H), 7.69 (d, J=8.8 Hz, 2H), 7.65-7.62 (m, 1H), 7.45 (d, J=8.4 Hz, 1H), 7.31 (s, 1H), 7.15 (d, J=7.6 Hz, 2H), 6.24-6.19 (m, 1H), 5.88-5.85 (m, 1H), 5.11 (d, J=8.4 Hz, 2H), 4.33-4.22 (m, 3H), 4.06-4.05 (m, 1H), 2.55 (dd, J=12.8, 2.0 Hz, 3H). MS: Calculated 651.1, found 652.0 ([M+H]⁺).

### Compound H6

The product (17.1 mg, 13.2%) was isolated with preparative high performance liquid chromatography as a white solid.

¹H-NMR (300 MHz, CD₃OD) δ 8.07 (d, J=8.7 Hz, 1H), 7.69 (d, J=8.7 Hz, 2H), 7.61-7.58 (m, 1H), 7.45 (d, J=8.2 Hz, 1H), 7.30 (s, 1H), 7.16 (d, J=8.1 Hz, 2H), 6.23-6.22 (m, 1H), 5.85 (d, J=7.5 Hz, 1H), 5.11 (d, J=8.4 Hz, 2H), 4.32-4.20 (m, Hz, 3H), 4.06-4.05 (m, 1H), 2.96-2.90 (m, 2H), 1.11 (t, J=6.7 Hz, 3H). MS: Calculated 665.1, found 666.0 ([M+H]⁺).

### Compound H7

The product (50.0 mg, 15.7%) was isolated with neutral preparative high performance liquid chromatography as a white solid.

¹H-NMR (400 MHz, CD₃OD) δ 8.03 (dd, J=8.4, 3.2 Hz, 1H), 7.59-7.41 (m, 5H), 7.32 (s, 1H), 7.18 (t, J=8.8 Hz, 2H), 6.93-6.91 (m, 2H), 6.21-6.16 (m, 1H), 5.86-5.84 (m, 1H), 5.11 (d, J=8.4 Hz, 2H), 4.30-4.24 (m, 3H), 4.05-4.0 (s, 1H), 2.72-2.68 (m, 2H), 1.74-1.61 (m, 1H), 0.88-0.86 (m, 6H). MS: Calculated 737.2, found 738.2 ([M+H]⁺).

### Compound H8

The product (49.5 mg, 15.3%) was isolated with neutral preparative high performance liquid chromatography as a white solid.

¹H-NMR (400 MHz, CD₃OD) δ 8.00 (dd, J=8.4, 3.2 Hz, 1H), 7.56-7.43 (m, 4H), 7.33-7.14 (m, 9H), 6.92-6.88 (m, 2H), 6.21-6.16 (m, 1H), 5.80-5.78 (m, 1H), 5.05-5.02 (m, 2H), 4.34-4.00 (m, 6H). MS: Calculated 771.2, found 772.1 ([M+H]⁺).

### Compound H9

The product (80.0 mg, yield 25.5%) was isolated with neutral preparative high performance liquid chromatography as a white solid.

¹H-NMR (400 MHz, CD₃OD) δ 8.03 (dd, J=8.4, 3.2 Hz, 1H), 7.67 (d, J=8.4 Hz, 2H), 7.54 (dd, J=16.0, 7.6 Hz, 1H), 7.37-7.35 (m, 1H), 7.31-7.20 (m, 6H), 7.12 (d, J=8.0 Hz, 2H), 6.22-6.17 (m, 1H), 5.80 (t, J=8.0 Hz, 1H), 5.08-5.02 (m, 2H), 4.35-4.10 (m, 3H), 4.10-4.01 (m, 3H). MS: Calculated MS, 727.1, found, 728.1 ([M+H]⁺).

### Compound H10

The product (65.1 mg, 21.3%) was isolated with neutral preparative high performance liquid chromatography as a white solid.

¹H-NMR (400 MHz, CD₃OD) δ 8.06 (dd, J=8.4, 3.2 Hz, 1H), 7.69 (d, J=8.7 Hz, 2H), 7.61-7.60 (m, 1H), 7.44 (d, J=8.5 Hz, 1H), 7.31 (s, 1H), 7.15 (d, J=8.4 Hz, 2H), 6.23-6.19 (m, 1H), 5.86 (dd, J=7.6, 2.4 Hz, 1H), 5.11 (d, J=8.0 Hz, 2H), 4.35-4.16 (m, 3H), 4.05-4.04 (m, 1H), 2.72-267 (m, 2H), 1.73-1.62 (m, 1H), 0.89-0.86 (m, 6H). MS: Calculated 693.2, found 694.1 ([M+H]⁺).

### Compound H11

The product (550 mg, 47.0%) was isolated with neutral preparative high performance liquid chromatography as a white solid.

¹H-NMR (400 MHz, CD₃OD) δ 8.04 (dd, J=8.4, 3.6 Hz, 1H), 7.60-7.48 (m, 4H), 7.39 (d, J=8.8 Hz, 1H), 7.31 (s, 1H), 7.18 (t, J=8.8 Hz, 2H), 6.94-6.91 (m, 2H), 6.26-6.13 (m, 1H), 5.84 (dd, J=7.6, 4.4 Hz, 1H), 5.12-5.09 (m, 2H), 4.34-4.17 (m, 3H), 4.04-4.01 (m, 1H), 3.37-3.31 (m, 1H), 1.15-1.12 (m, 6H). MS: Calculated MS, 723.2, found, 724.0 ([M+H]⁺).

### Compound H12

The product (24.6 mg, 8.3%) was isolated with neutral preparative high performance liquid chromatography as a white solid.

¹H-NMR (400 MHz, CD₃OD) δ 8.00 (dd, J=8.4, 2.8 Hz, 1H), 7.64-7.55 (m, 3H), 7.50-7.46 (m, 2H), 7.31-7.29 (m, 2H), 7.18-7.14 (m, 3H), 7.04-6.98 (m, 1H), 6.21-6.16 (m, 1H), 5.84-5.81 (m, 1H), 5.06 (d, J=8.0 Hz, 2H), 4.30-4.13 (m, 3H), 4.01-3.96 (m, 1H), 2.90-2.82 (m, 2H), 1.05 (t, J=6.8 Hz, 3H). MS: Calculated MS, 691.2, found, 692.2 ([M+H]⁺).

### Compound H13

The product (107.6 mg, yield 34.5%) was isolated with neutral preparative high performance liquid chromatography as a white solid.

¹H-NMR (400 MHz, CD₃OD) δ 8.03 (dd, J=8.4, 3.2 Hz, 1H), 7.60 (dd, J=7.4, 3.0 Hz, 1H), 7.44 (d, J=8.4 Hz, 2H), 7.42-7.38 (m, 1H), 7.26 (s, 1H), 7.09 (dd, J=8.6, 1.0 Hz, 2H), 6.24-6.19 (m, 1H), 5.84 (dd, J=7.6, 2.8 Hz, 1H), 5.09 (d, J=8.0 Hz, 2H), 4.36-4.15 (m, 3H), 4.05-4.02 (m, 1H), 3.77-3.64 (m, 2H), 3.55-3.37 (m, 2H), 2.97-2.89 (m, 2H), 1.75-1.51 (m, 6H), 1.13-1.09 (m, 3H). MS: Calculated MS, 708.2, found, 709.2 ([M+H]⁺).

### Compound H14

The product (71.5 mg, yield 18.1%) was isolated with preparative high performance liquid chromatography as a white solid.

¹H-NMR (400 MHz, CD₃OD) δ 8.04 (dd, J=8.4, 3.2 Hz, 1H), 7.61-7.58 (m, 1H), 7.48 (dd, J=6.8, 1.6 Hz, 2H), 7.40 (dd, J=8.4, 0.8 Hz, 1H), 7.27 (s, 1H), 7.10 (dd, J=8.4, 1.6 Hz, 2H), 6.24-6.18 (m, 1H), 5.84 (dd, J=7.6, 2.8 Hz, 1H), 5.13-5.08 (m, 2H), 4.80-4.60 (m, 1H), 4.33-4.14 (m, 3H), 4.05-4.03 (m, 1H), 4.03-3.70 (m, 1H), 3.23-3.13 (m, 1H), 2.97-2.89 (m, 3H), 2.60-2.45 (m, 1H), 2.08-1.84 (m, 2H), 1.62-1.48 (m, 2H), 1.14-1.09 (m, 3H). MS: Calculated MS, 776.2, found, 777.2 ([M+H]⁺).

### Compound H15

The product (25.2 mg, yield 13.2%) was isolated with preparative high performance liquid chromatography as a white solid.

¹H-NMR (400 MHz, CD₃OD) δ 8.02 (dd, J=8.4, 4.0 Hz, 1H), 7.60 (dd, J=7.6, 3.2 Hz, 1H), 7.49 (t, J=8.0 Hz, 1H), 7.38 (d, J=8.8 Hz, 1H), 7.26-7.18 (m, 2H), 7.15-7.13 (m, 1H), 7.02-7.01 (m, 1H), 6.25-6.15 (m, 1H), 5.84 (dd, J=7.6, 2.4 Hz, 1H), 5.08 (d, J=8.0 Hz, 2H), 4.33-4.15 (m, 3H), 4.05-4.01 (m, 1H), 3.72-3.65 (m, 2H), 3.40-3.30 (m, 2H), 2.95-2.85 (m, 2H), 1.75-1.45 (m, 6H), 1.13-1.08 (m, 3H). MS: Calculated MS, 708.2, found, 709.2 ([M+H]⁺).

### Compound H16

The product (110 mg, yield 33.7%) was isolated with preparative high performance liquid chromatography as a white solid.

¹H-NMR (400 MHz, CD₃OD) δ 8.02 (dd, J=8.4, 4.0 Hz, 1H), 7.60 (dd, J=7.6, 3.6 Hz, 1H), 7.50 (t, J=8.0 Hz, 1H), 7.38 (d, J=8.4 Hz, 1H), 7.24-7.20 (m, 2H), 7.17-7.12 (m, 1H), 7.05 (d, J=1.2 Hz, 1H), 6.26-6.18 (m, 1H), 5.83 (dd, J=7.6, 2.8 Hz, 1H), 5.08 (d, J=8.4 Hz, 2H), 4.77-4.63 (m, 1H), 4.33-4.15 (m, 3H), 4.05-4.02 (m, 1H), 3.82-3.73 (m, 1H), 3.18-3.10 (m, 1H), 2.97-2.80 (m, 3H), 2.60-2.42 (m, 1H), 2.05-1.83 (m, 2H), 1.60-1.40 (m, 2H), 1.14-1.08 (m, 3H). MS: Calculated MS, 776.2, found, 777.2 ([M+H]⁺).

### Compound H17

The product (39.6 mg, yield 11.1%) was isolated with preparative high performance liquid chromatography as a white solid.

¹H-NMR (400 MHz, CD₃OD) δ 8.04 (dd, J=8.4, 2.4 Hz, 1H), 7.62-7.45 (m, 4H), 7.40 (dd, J=8.4, 1.6 Hz, 1H), 7.32 (s, 1H), 7.18 (t, J=8.8 Hz, 2H), 6.95-6.91 (m, 2H), 6.26-6.16 (m, 1H), 5.84 (dd, J=7.6, 1.6 Hz, 1H), 5.11 (d, J=8.4 Hz, 2H), 4.35-4.16 (m, 3H), 4.05-4.00 (m, 1H), 2.96-2.85 (m, 1H), 1.91-1.85 (m, 2H), 1.76-1.64 (m, 2H), 1.58-1.50 (m, 1H), 1.30-1.07 (m, 5H). MS: Calculated MS, 763.2, found, 764.2 ([M+H]⁺).

### Compound H18

The product (36.1 mg, yield 15.9%) was isolated with preparative high performance liquid chromatography as a white solid.

¹H-NMR (400 MHz, CD₃OD) δ 8.07 (dd, J=8.4, 2.4 Hz, 1H), 7.69 (d, J=8.8 Hz, 2H), 7.61 (d, J=7.2 Hz, 1H), 7.44 (d, J=8.0 Hz, 1H), 7.31 (s, 1H), 7.15 (d, J=8.4 Hz, 2H), 6.26-6.17 (m, 1H), 5.86 (d, J=7.6 Hz, 1H), 5.11 (d, J=8.0 Hz, 2H), 4.33-4.15 (m, 3H), 4.06-4.00 (m, 1H), 2.95-2.87 (m, 1H), 1.90-1.82 (m, 2H), 1.75-1.62 (m, 2H), 1.60-1.51 (m, 1H), 1.30-1.09 (m, 5H). MS: Calculated MS, 719.2, found, 720.2 ([M+H]⁺).

### Compound H19

The product (5.4 mg, yield 2.8%) was isolated with preparative high performance liquid chromatography as a white solid.

¹H-NMR (400 MHz, CD₃OD) δ 8.81 (dd, J=4.4, 1.6 Hz, 1H), 8.28 (d, J=8.0 Hz, 1H), 8.12-8.03 (m, 2H), 7.62-7.51 (m, 3H), 7.47-7.45 (m, 1H), 7.41 (d, J=8.4 Hz, 1H), 7.29 (s, 1H), 6.20-6.11 (m, 1H), 5.83 (t, J=7.6 Hz, 1H), 5.09 (d, J=8.4 Hz, 2H), 4.29-4.12 (m, 3H), 3.97-3.90 (m, 1H), 2.90-2.80 (m, 2H), 1.06-1.00 (m, 3H). MS: Calculated MS, 648.2, found, 649.2 ([M+H]⁺).

### Compound H20

The product (23 mg, yield 11.5%) was isolated with preparative high performance liquid chromatography as a light yellow solid.

¹H-NMR (400 MHz, CD₃OD) δ 8.01 (dd, J=8.4, 2.4 Hz, 1H), 7.91 (d, J=8.8 Hz, 1H), 7.63 (t, J=2.4 Hz, 1H), 7.55 (dd, J=10.8, 7.6 Hz, 1H), 7.31 (d, J=8.4 Hz, 1H), 7.24 (dd, J=8.8, 1.2 Hz, 1H), 7.15 (s, 1H), 6.24-6.13 (m, 1H), 5.81 (t, J=7.8 Hz, 1H), 5.09-5.01 (m, 2H), 4.28-4.10 (m, 3H), 3.98-3.90 (m, 1H), 2.88-2.78 (m, 5H), 1.04 (t, J=6.4 Hz, 3H). MS: Calculated MS, 668.1, found, 669.1 ([M+H]⁺).

### Compound H21

The product (16.8 mg, yield 16.5%) was isolated with preparative high performance liquid chromatography as a white solid.

¹H-NMR (400 MHz, CD₃OD) δ 8.08-8.01 (m, 1H), 7.69-7.37 (m, 5H), 7.31-7.30 (m, 1H), 7.18 (t, J=9.6 Hz, 2H), 6.95-6.87 (m, 2H), 6.27-6.11 (m, 1H), 5.90-5.82 (m, 1H), 5.53-5.42 (m, 1H), 4.38-3.92 (m, 4H), 2.97-2.73 (m, 2H), 1.64-1.59 (m, 3H), 1.15-1.00 (m, 3H). MS: Calculated MS, 723.2, found, 724.2 ([M+H]⁺).

### Compound H22

The product (54.0 mg, yield 22.5%) was isolated with preparative high performance liquid chromatography as a light yellow solid.

¹H-NMR (400 MHz, CD₃OD) δ 8.08-8.01 (m, 1H), 7.63-7.40 (m, 5H), 7.33-7.30 (m, 1H), 7.18 (t, J=8.8 Hz, 2H), 6.95-6.90 (m, 2H), 6.25-6.10 (m, 1H), 5.90-5.82 (m, 1H), 5.59-5.50 (m, 1H), 4.40-3.92 (m, 4H), 3.49-3.12 (m, 4H), 1.67-1.59 (m, 3H). MS: Calculated MS, 801.1, 803.1, found, 802.0, 804.0 ([M+H]⁺).

### Compound H23

The product (5.7 mg, yield 5.0%) was isolated with preparative high performance liquid chromatography as a white solid.

¹H-NMR (400 MHz, CD₃OD) δ 8.10-8.03 (m, 1H), 7.75-7.45 (m, 4H), 7.34-7.30 (m, 1H), 7.16-7.11 (m, 2H), 6.27-6.13 (m, 1H), 5.89-5.82 (m, 1H), 5.55-5.46 (m, 1H), 4.38-3.95 (m, 4H), 2.60-2.40 (m, 3H), 1.65-1.60 (m, 3H). MS: Calculated MS, 665.1, found, 666.2 ([M+H]⁺).

### Compound H24

The product (6.3 mg, yield 5.0%) was isolated with preparative high performance liquid chromatography as a white solid.

¹H-NMR (400 MHz, CD₃OD) δ 8.08-8.01 (m, 1H), 7.69-7.52 (m, 1H), 7.47-7.40 (m, 3H), 7.26 (s, 1H), 7.13-7.05 (m, 2H), 6.28-6.15 (m, 1H), 5.91-5.82 (m, 1H), 5.52-5.42 (m, 1H), 4.35-3.95 (m, 4H), 3.76-3.68 (m, 2H), 3.45-3,32 (m, 2H), 2.98-2.75 (m, 2H), 1.78-1.50 (m, 9H), 1.17-1.02 (m, 3H). MS: Calculated MS, 722.2, found, 723.2 ([M+H]⁺).

### Compound H25

The product (4.7 mg, yield 3.8%) was isolated with preparative high performance liquid chromatography as a white solid.

¹H-NMR (400 MHz, CD₃OD) δ 8.06-7.98 (m, 1H), 7.67-7.35 (m, 3H), 7.27-7.10 (m, 3H), 7.01 (s, 1H), 6.28-6.13 (m, 1H), 5.90-5.80 (m, 1H), 5.52-5.42 (m, 1H), 4.38-3.95 (m, 4H), 3.73-3.63 (m, 2H), 3.42-3.32 (m, 2H), 2.97-2.73 (m, 2H), 1.76-1.48 (m, 9H), 1.17-1.00 (m, 3H). MS: Calculated MS, 722.2, found, 723.3 ([M+H]⁺).

### Compound H27

The product (64.3 mg, yield 29.6%) was isolated with preparative high performance liquid chromatography as a white solid.

¹H-NMR (400 MHz, CD₃OD) δ 8.41 (t, J=2.0 Hz, 1H), 8.07 (dd, J=8.4, 3.2 Hz, 1H), 8.03-7.98 (m, 2H), 7.88 (d, J=8.8 Hz, 1H), 7.60 (t, J=8.0 Hz, 1H), 7.57-7.52 (m, 1H), 7.42 (dd, J=8.4, 1.6 Hz, 1H), 7.30 (d, J=1.6 Hz, 1H), 7.21 (t, J=8.8 Hz, 2H), 6.25-6.15 (m, 1H), 5.88-5.83 (m, 1H), 5.16-5.07 (m, 2H), 4.35-4.15 (m, 3H), 4.07-3.98 (m, 1H), 2.96-2.85 (m, 2H), 1.13-1.05 (m, 3H). MS: Calculated MS, 692.2, found, 692.7 ([M+H]⁺).

### Compound H28

The product (23.8 mg, yield 19.2%) was isolated with preparative high performance liquid chromatography as a white solid.

¹H-NMR (400 MHz, CD₃OD) δ 8.51 (d, J=2.8 Hz, 1H), 8.05-8.01 (m, 3H), 7.90 (dd, J=8.8, 4.0 Hz, 1H), 7.70-7.62 (m, 1H), 7.59 (dd, J=9.8, 7.6 Hz, 1H), 7.34 (d, J=8.4 Hz, 1H), 7.23 (s, 1H), 7.165 (dd, J=8.8, 2.0 Hz, 2H), 6.25-6.15 (m, 1H), 5.83 (t, J=6.8 Hz, 1H), 5.14-5.03 (m, 2H), 4.31-4.13 (m, 3H), 4.05-3.97 (m, 1H), 2.94-2.85 (m, 2H), 1.12-1.05 (m, 3H). MS: Calculated MS, 692.2, found, 692.7 ([M+H]⁺).

### Compound H29

The product (3 mg, yield 1.5%) was isolated with preparative high performance liquid chromatography as a white solid.

¹H-NMR (400 MHz, CD₃OD) δ 8.04 (dd, J=8.4, 3.2 Hz, 1H), 7.60-7.47 (m, 4H), 7.39 (d, J=8.4 Hz, 1H), 7.32 (s, 1H), 7.18 (t, J=8.8 Hz, 2H), 6.95-6.91 (m, 2H), 6.27-6.17 (m, 1H), 5.83 (dd, J=7.6, 4.4 Hz, 1H), 5.10 (d, J=8.0 Hz, 2H), 4.38-4.18 (m, 3H), 4.07-4.01 (m, 1H), 3.53-3.43 (m, 1H), 1.90-1.78 (m, 2H), 1.75-1.63 (m, 2H), 1.58-1.38 (m, 4H). MS: Calculated MS, 749.2, found, 750.2 ([M+H]⁺).

### Compound H30

The product (45 mg, yield 26.0%) was isolated with preparative high performance liquid chromatography as a white solid.

¹H-NMR (400 MHz, CD₃OD) δ 8.04 (dd, J=8.4, 3.2 Hz, 1H), 7.65-7.46 (m, 4H), 7.39 (d, J=8.4 Hz, 1H), 7.30 (s, 1H), 7.18 (t, J=8.8 Hz, 2H), 6.93 (dd, J=10.8, 2.0 Hz, 2H), 6.27-6.17 (m, 1H), 5.90-5.83 (m, 1H), 5.15-5.04 (m, 2H), 4.30-4.14 (m, 3H), 4.07-4.02 (m, 1H), 3.68-3.52 (m, 1H), 2.25-2.10 (m, 2H), 1.97-1.83 (m, 2H), 1.67-1.43 (m, 2H). MS: Calculated MS, 735.2, found, 736.1 ([M+H]⁺).

### Compound H31

The product (79 mg, yield 37.1%) was isolated with preparative high performance liquid chromatography as a white solid.

¹H-NMR (400 MHz, CD₃OD) δ 8.04 (dd, J=8.4, 4.8 Hz, 1H), 7.65-7.46 (m, 4H), 7.41 (d, J=8.4 Hz, 1H), 7.32 (s, 1H), 7.18 (t, J=8.8 Hz, 2H), 6.93-6.88 (m, 2H), 6.27-6.17 (m, 1H), 5.90-5.81 (m, 1H), 5.19-5.10 (m, 2H), 4.40-4.19 (m, 3H), 4.08-4.02 (m, 1H), 3.68-3.52 (m, 2H). MS: Calculated MS, 763.1, found, 764.0 ([M+H]⁺).

### Compound H32

The product (15.4 mg, yield 13.9%) was isolated with preparative high performance liquid chromatography as a white solid.

¹H-NMR (400 MHz, CD₃OD) δ 8.03 (dd, J=8.4, 5.2 Hz, 1H), 7.60-7.48 (m, 4H), 7.43-7.38 (m, 1H), 7.31 (s, 1H), 7.18 (t, J=8.8 Hz, 2H), 6.95-6.88 (m, 2H), 6.24-6.16 (m, 1H), 5.86-5.82 (m, 1H), 5.17-5.10 (m, 2H), 4.37-4.18 (m, 3H), 4.08-4.00 (m, 1H), 1.27 (s, 9H). MS: Calculated MS, 737.2, found, 738.1 ([M+H]⁺).

### Compound H33

The product (5.5 mg, yield 2.7%) was isolated with preparative high performance liquid chromatography as a white solid.

¹H-NMR (400 MHz, CD₃OD) S 8.04 (dd, J=8.4, 3.6 Hz, 1H), 7.67-7.45 (m, 4H), 7.40 (d, J=8.4 Hz, 1H), 7.32 (s, 1H), 7.18 (t, J=8.8 Hz, 2H), 6.92 (dd, J=10.4, 1.6 Hz, 2H), 6.25-6.15 (m, 1H), 5.85 (dd, J=7.6, 4.4 Hz, 1H), 5.18-5.13 (m, 2H), 4.38-4.18 (m, 3H), 4.08-4.00 (m, 1H), 2.68 (d, J=10.4 Hz, 2H), 0.89-0.84 (m, 9H). MS: Calculated MS, 751.2, found, 752.2 ([M+H]⁺).

### Compound H34

The product (35.8 mg, yield 24.9%) was isolated with preparative high performance liquid chromatography as a white solid.

¹H-NMR (400 MHz, CD₃OD) δ 8.04 (dd, J=8.4, 3.2 Hz, 1H), 7.66-7.45 (m, 4H), 7.40 (d, J=8.4 Hz, 1H), 7.32 (s, 1H), 7.18 (t, J=8.8 Hz, 2H), 6.99-6.91 (m, 2H), 6.28-6.15 (m, 1H), 5.84 (t, J=8.0 Hz, 1H), 5.20-5.10 (m, 2H), 4.40-4.18 (m, 3H), 4.08-4.00 (m, 1H), 2.38-2.27 (m, 1H), 0.58-0.45 (m, 4H). MS: Calculated MS, 721.2, found, 722.1 ([M+H]⁺).

### Compound H35

The product (36.7 mg, yield 12.3%) was isolated with preparative high performance liquid chromatography as a white solid.

¹H-NMR (400 MHz, CD₃OD) δ 8.02 (dd, J=8.4, 4.4 Hz, 1H), 7.60 (d, J=7.6 Hz, 1H), 7.49 (t, J=8.0 Hz, 1H), 7.37 (d, J=8.4 Hz, 1H), 7.26-7.18 (m, 2H), 7.14 (dd, J=8.0, 2.4 Hz, 1H), 7.02 (d, J=1.6 Hz, 1H), 6.28-6.21 (m, 1H), 5.90-5.83 (m, 1H), 5.08 (d, J=8.0 Hz, 2H), 4.38-4.25 (m, 3H), 4.08-4.02 (m, 1H), 3.74-3.65 (m, 2H), 3.40-3.34 (m, 3H), 1.78-1.45 (m, 6H), 1.19-1.12 (m, 6H). MS: Calculated MS, 722.3, found, 723.2 ([M+H]⁺).

### Compound H36

The product (26.6 mg, yield 8.9%) was isolated with preparative high performance liquid chromatography as a white solid.

¹H-NMR (400 MHz, CD₃OD) δ 8.03 (dd, J=8.4, 3.6 Hz, 1H), 7.60 (dd, J=7.6, 2.4 Hz, 1H), 7.44 (d, J=8.8 Hz, 2H), 7.39 (d, J=8.4 Hz, 1H), 7.26 (s, 1H), 7.09 (dd, J=8.8, 1.2 Hz, 2H), 6.28-6.16 (m, 1H), 5.84 (dd, J=7.6, 2.8 Hz, 1H), 5.08 (d, J=8.0 Hz, 2H), 4.35-4.15 (m, 3H), 4.08-4.02 (m, 1H), 3.76-3.62 (m, 2H), 3.50-3.40 (m, 2H), 3.38-3.32 (m, 1H), 1.78-1.49 (m, 6H), 1.18-1.12 (m, 6H). MS: Calculated MS, 722.2, found, 723.2 ([M+H]⁺).

### Compound H38

The product (17.6 mg, yield 20.7%) was isolated with preparative high performance liquid chromatography as a white solid.

¹H-NMR (400 MHz, DMSO-d₆) δ 8.11 (dd, J=8.4, 1.2 Hz, 1H), 7.73-7.67 (m, 4H), 7.51 (d, J=7.2 Hz, 1H), 7.45-7.37 (m, 3H), 7.29 (t, J=8.8 Hz, 2H), 7.24-7.21 (m, 1H), 7.15 (d, J=8.0 Hz, 2H), 6.42-6.36 (m, 1H), 6.20-6.11 (m, 1H), 5.72 (dd, J=7.6, 2.0 Hz, 1H), 5.23-5.14 (m, 1H), 5.00 (d, J=8.0 Hz, 2H), 4.20-4.08 (m, 3H), 3.97-3.92 (m, 1H), 3.20-3.10 (m, 1H), 1.00-0.93 (m, 6H). MS: Calculated MS, 705.2, found, 706.2 ([M+H]⁺).

### Compound H39

The product (17.6 mg, yield 20.7%) was isolated with preparative high performance liquid chromatography as a white solid.

¹H-NMR (400 MHz, DMSO-d₆) δ 8.13 (dd, J=8.4, 0.8 Hz, 1H), 7.91-7.86 (m, 2H), 7.84-7.78 (m, 4H), 7.51 (d, J=7.6 Hz, 1H), 7.45-7.37 (m, 3H), 7.27 (d, J=2.0 Hz, 1H), 7.19 (d, J=8.4 Hz, 2H), 6.42-6.36 (m, 1H), 6.20-6.10 (m, 1H), 5.73 (dd, J=7.6, 2.0 Hz, 1H), 5.23-5.12 (m, 1H), 5.02 (d, J=8.0 Hz, 2H), 4.20-4.08 (m, 3H), 3.98-3.92 (m, 1H), 3.20-3.10 (m, 1H), 1.02-0.95 (m, 6H). MS: Calculated MS, 755.2, found, 756.2 ([M+H]⁺).

### Compound H40

The product (48 mg, yield 30.2%) was isolated with preparative high performance liquid chromatography as a white solid.

¹H-NMR (400 MHz, DMSO-d₆) δ 8.12 (dd, J=8.4, 1.2 Hz, 1H), 7.77-7.66 (m, 4H), 7.60-7.35 (m, 6H), 7.25-7.22 (m, 1H), 7.16 (d, J=8.4 Hz, 2H), 6.40 (t, J=6.8 Hz, 1H), 6.20-6.10 (m, 1H), 5.74 (dd, J=7.6, 2.0 Hz, 1H), 5.23-5.12 (m, 1H), 5.01 (d, J=8.0 Hz, 2H), 4.20-4.08 (m, 3H), 3.98-3.92 (m, 1H), 3.20-3.10 (m, 1H), 1.02-0.95 (m, 6H). MS: Calculated MS, 721.2, 723.2, found, 721.9, 723.9 ([M+H]⁺).

### Compound H41

The product (6.5 mg, yield 4.2%) was isolated with preparative high performance liquid chromatography as a white solid.

¹H-NMR (400 MHz, DMSO-d₆) δ 8.11 (dd, J=8.4, 0.8 Hz, 1H), 7.71 (dd, J=8.8, 0.8 Hz, 2H), 7.68-7.63 (m, 2H), 7.58-7.34 (m, 7H), 7.23 (s, 1H), 7.16 (d, J=8.4 Hz, 2H), 6.43-6.38 (m, 1H), 6.20-6.10 (m, 1H), 5.73 (dd, J=7.6, 2.4 Hz, 1H), 5.23-5.15 (m, 1H), 5.01 (d, J=7.6 Hz, 2H), 4.25-4.10 (m, 3H), 3.98-3.92 (m, 1H), 3.20-3.10 (m, 1H), 1.12-0.96 (m, 6H). MS: Calculated MS, 687.2, found, 687.8 ([M+H]⁺).

### Compound H42

The product (61 mg, yield 20.4%) was isolated with preparative high performance liquid chromatography as a white solid.

¹H-NMR (400 MHz, DMSO-d₆) b 8.15 (dd, J=8.4, 1.6 Hz, 1H), 7.60-7.50 (m, 6H), 7.49-7.38 (m, 3H), 7.34 (s, 1H), 7.14 (dd, J=11.6, 1.2 Hz, 1H), 6.98 (dd, J=8.8, 2.4 Hz, 1H), 6.40 (t, J=6.8 Hz, 1H), 6.20-6.10 (m, 1H), 5.74 (dd, J=7.6, 2.4 Hz, 1H), 5.24-5.13 (m, 1H), 5.04 (d, J=8.0 Hz, 2H), 4.25-4.10 (m, 3H), 3.98-3.92 (m, 1H), 3.20-3.10 (m, 1H), 1.04-0.95 (m, 6H). MS: Calculated MS, 739.1, 741.1, found, 739.9, 741.9 ([M+H]⁺).

### Compound H43

The product (54 mg, yield 23.1%) was isolated with preparative high performance liquid chromatography as a white solid.

¹H-NMR (400 MHz, DMSO-d₆) δ 8.12 (dd, J=8.4, 1.2 Hz, 1H), 7.60 (d, J=7.6 Hz, 2H), 7.61-7.37 (m, 6H), 7.36-7.28 (m, 3H), 7.17 (d, J=8.8 Hz, 2H), 6.40 (t, J=6.8 Hz, 1H), 6.20-6.10 (m, 1H), 5.74 (dd, J=7.6, 2.8 Hz, 1H), 5.25-5.15 (m, 1H), 5.03 (d, J=7.6 Hz, 2H), 4.25-4.09 (m, 3H), 3.99-3.95 (m, 1H), 3.20-3.09 (m, 1H), 1.02-0.95 (m, 6H). MS: Calculated MS, 705.2, found, 706.0 ([M+H]⁺).

### Compound H44

The product (55 mg, yield 18.6%) was isolated with preparative high performance liquid chromatography as a white solid.

¹H-NMR (400 MHz, DMSO-d₆) δ 8.12 (dd, J=8.4, 1.2 Hz, 1H), 7.76 (d, J=8.4 Hz, 2H), 7.55-7.34 (m, 7H), 7.24 (s, 1H), 7.23-7.10 (m, 3H), 6.41 (t, J=6.4 Hz, 1H), 6.20-6.10 (m, 1H), 5.73 (dd, J=7.6, 2.8 Hz, 1H), 5.20-5.10 (m, 1H), 5.02 (d, J=7.6 Hz, 2H), 4.23-4.08 (m, 3H), 3.99-3.95 (m, 1H), 3.20-3.08 (m, 1H), 1.03-0.94 (m, 6H). MS: Calculated MS, 705.2, found, 705.9 ([M+H]⁺).

### Compound D1

The product (15 mg, yield 9.6%) was isolated with preparative high performance liquid chromatography as a white solid.

¹H-NMR (400 MHz, CD₃OD) δ 8.05 (dd, J=8.4, 6.4 Hz, 1H), 7.61-7.50 (m, 4H), 7.45-7.40 (m, 1H), 7.30 (d, J=2.0 Hz, 1H), 7.18 (t, J=8.8 Hz, 2H), 6.96-6.90 (m, 2H), 6.27-6.16 (m, 1H), 5.88-5.83 (m, 1H), 5.19 (dd, J=8.8, 2.4 Hz, 2H), 4.43-4.30 (m, 2H), 4.28-4.13 (m, 3H), 4.07-4.02 (m, 1H), 1.38-1.27 (m, 3H). MS: Calculated MS, 710.1, found, 710.7 ([M+H]⁺).

### Compound D2

The product (4.5 mg, 4.1%) was isolated with neutral preparative high performance liquid chromatography as a white solid.

¹H-NMR (400 MHz, CD₃OD) δ 8.06-8.03 (m, 1H), 7.56-7.39 (m, 5H), 7.28 (s, 1H), 7.17 (t, J=8.8 Hz, 2H), 6.95-6.92 (m, 2H), 6.23-6.15 (m, 1H), 5.85 (t, J=7.2 Hz, 1H), 5.12-5.05 (m, 2H), 4.31-4.14 (m, 3H), 4.05-4.03 (m, 1H), 3.12-3.06 (m, 4H), 1.11-107 (m, 6H). MS: Calculated 737.2, found 738.0 ([M+H]⁺).

### Compound D3

The product (6.6 mg, yield 21.10%) was isolated with neutral preparative high performance liquid chromatography as a white solid.

¹H-NMR (300 MHz, CD₃OD) δ 8.05 (d, J=7.8 Hz, 1H), 7.61-7.39 (m, 5H), 7.30 (s, 1H), 7.18 (t, J=8.7 Hz, 2H), 6.95-6.92 (m, 2H), 6.25-6.16 (m, 1H), 5.86-5.82 (m, 1H), 5.10 (d, J=8.7 Hz, 2H), 4.28-4.14 (m, 3H), 4.04 (m, 1H), 2.68 (d, J=10.2 Hz, 6H). MS: Calculated 709.2, found 710.0 ([M+H]⁺).

### Compound D4

The product (14.2 mg, 6.9%) was isolated with neutral preparative high performance liquid chromatography as a white solid.

¹H-NMR (400 MHz, CD₃OD) δ 8.05 (t, J=8.0 Hz, 1H), 7.60-7.47 (m, 4H), 7.40 (t, J=7.2 Hz, 1H), 7.30 (d, J=4.4 Hz, 1H), 7.18 (t, J=8.8 Hz, 2H), 6.96-6.92 (m, 2H), 6.22-6.20 (m, 1H), 5.87-5.84 (m, 1H), 5.24-5.20 (m, 2H), 4.55-4.33 (m, 2H), 4.24-4.15 (m, 1H), 4.05-4.03 (m, 1H), 2.22 (d, J=16.0 Hz, 4H). MS: Calculated 707.2, found 707.9 ([M+H]⁺).

### Compound D5

The product (53.5 mg, 26.7%) was isolated with neutral preparative high performance liquid chromatography as a white solid.

¹H-NMR (400 MHz, CD₃CD₃OD) δ 8.07 (dd, J=8.4, 3.2 Hz, 1H), 7.70 (d, J=8.8 Hz, 2H), 7.60 (t, J=7.2 Hz, 1H), 7.45 (d, J=8.4 Hz, 1H), 7.29 (s, 1H), 7.17 (d, J=7.6 Hz, 2H), 6.25-6.18 (m, 1H), 5.87-5.84 (m, 1H), 5.12-5.08 (m, 2H), 4.30-4.23 (m, 3H), 4.06-4.05 (m, 1H), 2.70-2.66 (m, 6H). MS: Calculated 665.1, found 666.0 ([M+H]⁺).

### Compound D6

The product (45.0 mg, 19.1%) was isolated with neutral preparative high performance liquid chromatography as a white solid.

¹H-NMR (400 MHz, CD₃OD) δ 8.04-8.00 (m, 1H), 7.55-7.16 (m, 13H), 6.92-6.97 (m, 2H), 6.24-6.17 (m, 1H), 5.80-5.76 (m, 1H), 5.32-5.28 (m, 2H), 4.56-4.42 (m, 2H), 4.23-4.15 (m, 1H), 4.07-4.05 (m, 1H). MS: Calculated 758.1, found 759.1 ([M+H]⁺).

### Compound D7

The product (92.0 mg, 29.3%) was isolated with neutral preparative high performance liquid chromatography as a white solid.

¹H-NMR (400 MHz, CD₃OD) δ 8.04 (dd, J=8.4, 2.8 Hz, 1H), 7.67 (d, J=8.8 Hz, 2H), 7.43-7.33 (m, 4H), 7.25-7.17 (m, 4H), 7.12 (d, J=8.0 Hz, 2H), 6.23-6.16 (m, 1H), 5.78 (t, J=8.0 Hz, 1H), 5.30 (dd, J=9.2, 6.4 Hz, 2H), 4.56-4.43 (m, 2H), 4.23-4.16 (m, 1H), 4.07-4.06 (m, 1H). MS: Calculated 714.1, found 715.0 ([M+H]⁺).

### Compound D9

The product (4.0 mg, yield 3.6%) was isolated with neutral preparative high performance liquid chromatography as a white solid.

¹H NMR (400 MHz, CD₃OD) δ 8.10-8.02 (m, 1H), 7.69-7.32 (m, 5H), 7.30 (s, 1H), 7.18 (t, J=8.8 Hz, 2H), 6.97-6.90 (m, 2H), 6.28-6.10 (m, 1H), 5.92-5.80 (m, 1H), 5.50-5.43 (m, 1H), 4.30-3.92 (m, 4H), 2.70 (t, J=10.2 Hz, 3H), 2.56 (d, J=10.4 Hz, 3H), 1.63-1.59 (m, 3H). MS: Calculated MS, 723.2, found, 724.2 ([M+H]⁺).

1. A gemcitabine derivative of formula (III), or a pharmaceutically acceptable salt, a solvate, an isotopic variant, or an isomer thereof: 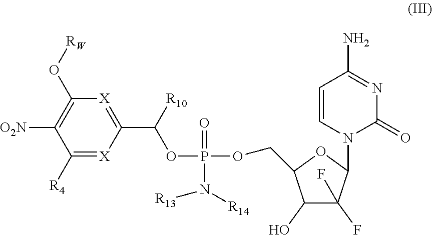
wherein,
two X's are each independently CR¹⁵ or N;
R₁₃ and R₁₄ are each independently hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl, and R₁₃ and R₁₄ are not hydrogen at the same time;
R₁₀ is hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
or R₁₀ and R₁₃ or R₁₄ may be connected to form a 5-9-membered ring under the condition that they meet the above definitions for R₁₀, R₁₃ and R₁₄;
R₄ and R¹⁵ are each independently hydrogen; halogen; C₁-C₆ alkyl; cycloalkyl; alkenyl; alkynyl; alkoxy; cyano; 5-20-membered heterocyclyl; C₆-C₂₀ aryl; or halogen-substituted C₁-C₆ alkyl, cycloalkyl, alkenyl, alkynyl, alkoxyl, 5-20-membered heterocyclyl or C₆-C₂₀ aryl;
or R₁₀ and R¹⁵ may form a 4-12-membered cyclic hydrocarbon or heterocycle under the condition that they meet the above definitions for R₁₀ and R¹⁵;
R_{W} is 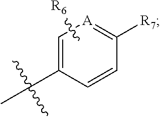
A is CR¹⁶ or N, and the position of A may be changed on the ring;
R¹⁶ is hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
R₆ and R₇ meet the following conditions:
R₆ and R₇ are each independently hydrogen; halogen; cyano; hydroxyl; C₁-C₆ alkyl, cycloalkyl, alkenyl, alkynyl, alkoxyl, 5-20-membered heterocyclyl or C₆-C₂₀ aryl; or halogen-substituted C₁-C₆ alkyl, cycloalkyl, alkenyl, alkynyl, alkoxyl, 5-20-membered heterocyclyl or C₆-C₂₀ aryl; or cyano-substituted C₁-C₆ alkyl, cycloalkyl, alkenyl, alkynyl, alkoxyl, 5-20-membered heterocyclyl or C₆-C₂₀ aryl; or hydroxyl-substituted C₁-C₆ alkyl, cycloalkyl, alkoxyl, 5-20-membered heterocyclyl or C₆-C₂₀ aryl; or —CONR¹¹R¹²; or —CH₂NR¹¹R¹²;
or R₆ and R₇ are connected to form
a 5-8-membered single or fused heterocycle containing at least one N or S or O, or two or three of N, S and O at the same time;
or a 5-8-membered single or fused heterocycle containing at least one N or S or O, or two or three of N, S and O at the same time, which is C₁-C₆ alkyl-substituted;
R₆ and CR¹⁶ may be connected to form a 5-9-membered ring, heterocycle, or aromatic heterocycle;
R¹¹ and R¹² meet the following conditions:
R¹¹ and R¹² are each independently C₁-C₆ alkyl or halogen-substituted C₁-C₆ alkyl, or R¹¹ and R¹² form a 5-7-membered ring with N from —CONR¹¹R¹², or form a 5-7-membered ring with N from —CH₂NR¹¹R¹² under the condition that they meet the above definitions for R¹¹ and R¹².

2. The compound according to claim 1, wherein the compound with the structural formula of formula (III) includes compounds of formula (I) and formula (II) below: 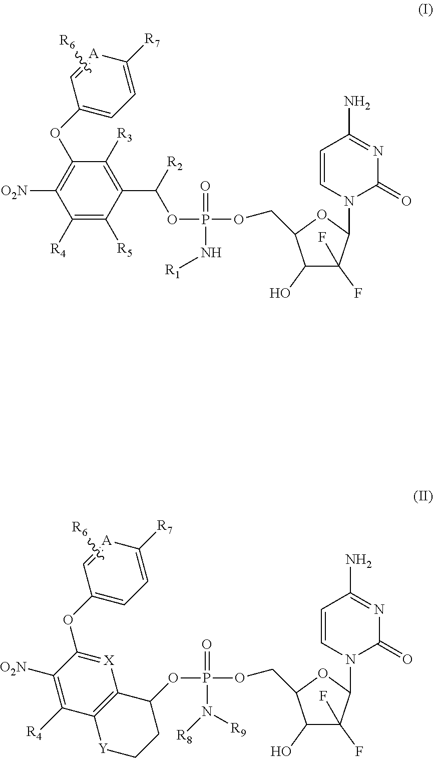
wherein,
Y is CR^{a}R^{b}, NR^{a}, or O, and the position of Y may be changed between the 4 positions excluding 2 carbon atoms shared with the benzene ring;
R^{a} and R^{b} meet the following conditions:
R^{a} and R^{b} are each independently hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
R₁, R₂, R₈ and R₉ meet the following conditions:
R₁ is C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
R₂ is hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl;
or R₁ and R₂ may be connected to form a 5-9-membered ring under the condition that they meet the above definitions for R₁ and R₂;
R₈ and R₉ are each independently hydrogen; C₁-C₆ alkyl; cycloalkyl; alkenyl; alkynyl; C₆-C₂₀ aryl; 5-20-membered heterocyclyl; halogen-substituted C₁-C₆ alkyl, cycloalkyl, alkenyl or alkynyl; halogen-substituted C₆-C₂₀ aryl; or halogen-substituted 5-20-membered heterocyclyl, and R₈ and R₉ are not hydrogen at the same time;
or R₈ and R₉ may be connected to form a 5-9-membered ring under the condition that they meet the above definitions for R₈ and R₉;
R₃, R₄ and R₅ meet the following conditions:
R₃, R₄ and R₅ are each independently hydrogen; halogen; C₁-C₆ alkyl; cycloalkyl; alkenyl; alkynyl; alkoxy; cyano; 5-20-membered heterocyclyl; C₆-C₂₀ aryl; or halogen-substituted C₁-C₆ alkyl, cycloalkyl, alkenyl, alkynyl, alkoxyl, 5-20-membered heterocyclyl or C₆-C₂₀ aryl.

3. The compound according to claim 2, wherein,
R₁, R₈ and R₉ are each independently C₁-C₅ alkyl, halogen-substituted C₁-C₅ alkyl, C₃-C₆ cycloalkyl or C₆-C₂₀ aryl.

4. The compound according to claim 2, wherein,
R₁, R₈ and R₉ are each independently methyl, ethyl, n-propyl, isopropyl, isobutyl, t-butyl, neopentyl, bromoethyl, 1,1,1-trifluoroethyl, C₃-C₆ cycloalkyl or benzyl;
R₂ is hydrogen, methyl, or trifluoromethyl.

5. The compound according to claim 2, wherein, R₁ and R₂ in formula (I) are connected to form a 6-membered ring.

6. The compound according to claim 2, wherein, R₃, R₄ and R₅ are each independently hydrogen.

7. The compound according to claim 2, wherein,
R₆ is hydrogen, fluorine, fluorophenyl, or

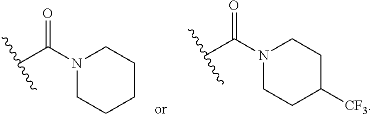 [SMILES: *C(=O)N1CCCCC1 ; "or" ; *C(=O)N1CCC(C(F)(F)F)CC1]

8. The compound according to claim 2, wherein,
R₇ is hydrogen, —CF₃, phenyl, chlorophenyl, fluorophenyl, —CF₃-substituted phenyl, fluoropyridyl, or

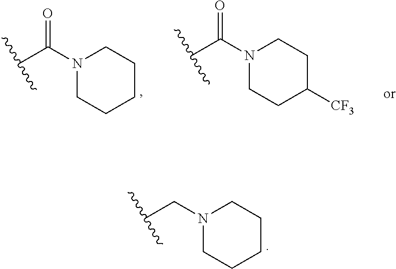 [SMILES: *C(=O)N1CCCCC1 ; "," ; *C(=O)N1CCC(C(F)(F)F)CC1 ; "or" ; *CN1CCCCC1 ; "."]

9. The compound according to claim 2, wherein,
R₆ and R₇ are connected to form a 5-8-membered heterocycle containing 2 N atoms, or C₁-C₆ alkyl-substituted 5-8-membered heterocycle, and preferably, R₆ and R₇ are connected to form a pyrimidine; or
R₆ and R₇ are connected to form a 5-8-membered heterocycle containing 1 N atom, or C₁-C₆ alkyl-substituted 5-8-membered heterocycle, and preferably, R₆ and R₇ are connected to form a pyridine.

10. The compound according to claim 2, wherein the compound with the structural formula of formula (I) includes compounds with the structures of formulas I-1 to I-5 below, and the compound with the structural formula of formula (II) includes compounds with the structures of formulas II-1 to II-6 below:

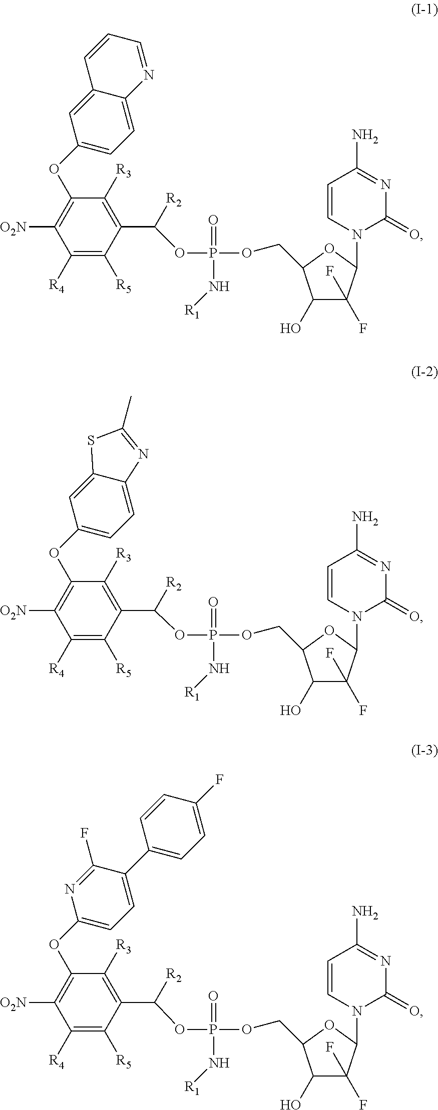

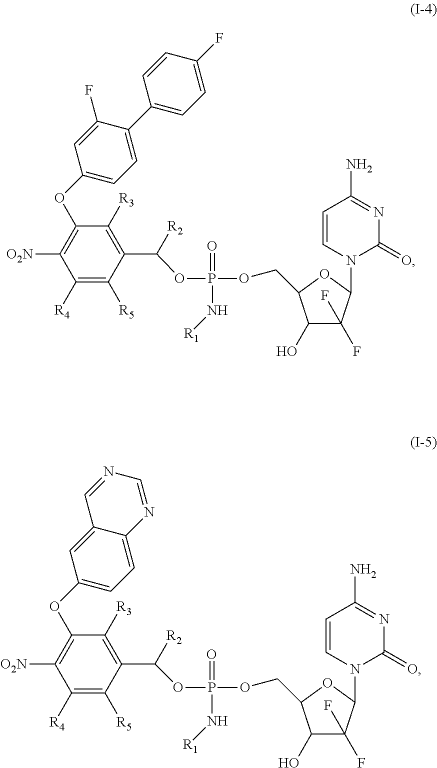

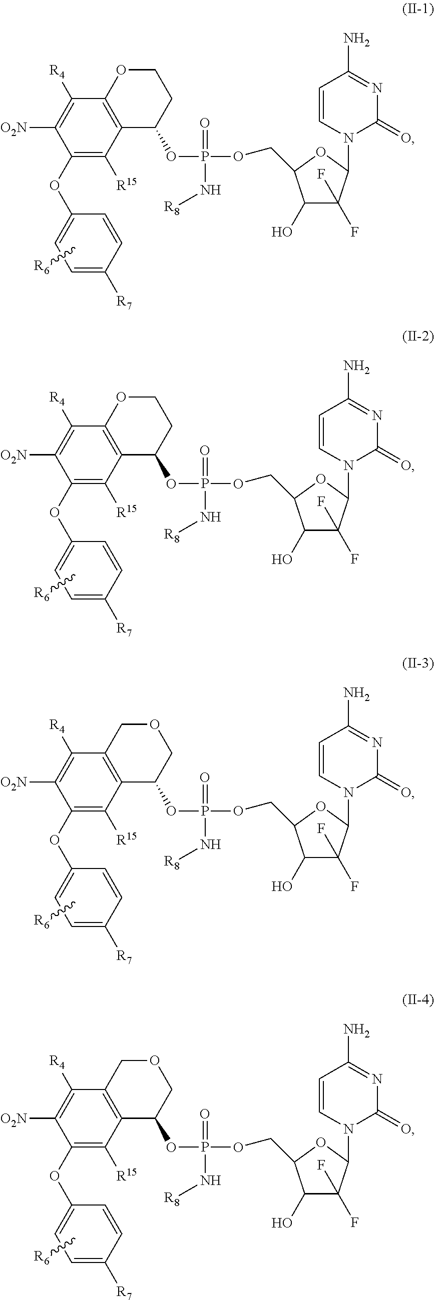

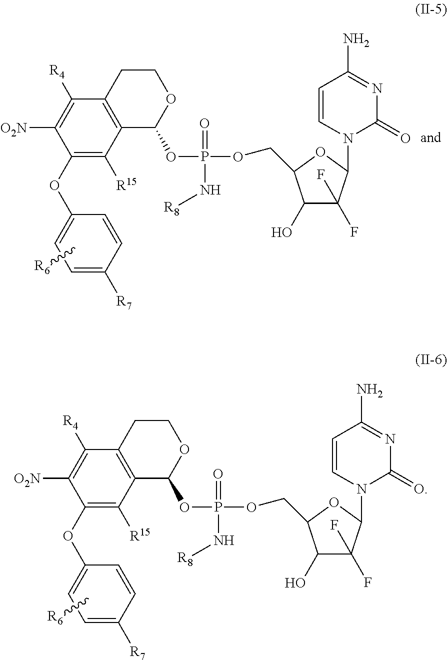

11. The compound according to claim 2, wherein the structure of formula (I) substituted by isotope deuterium includes compounds with the structures of formulas I-6 to I-13 below, and the structure of formula (II) substituted by isotope deuterium includes compounds with the structures of formulas II-7 to II-12 below:

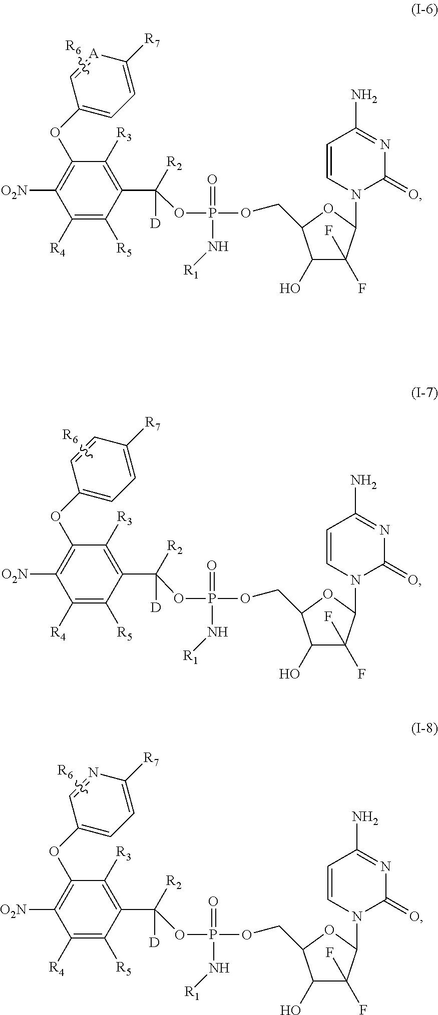

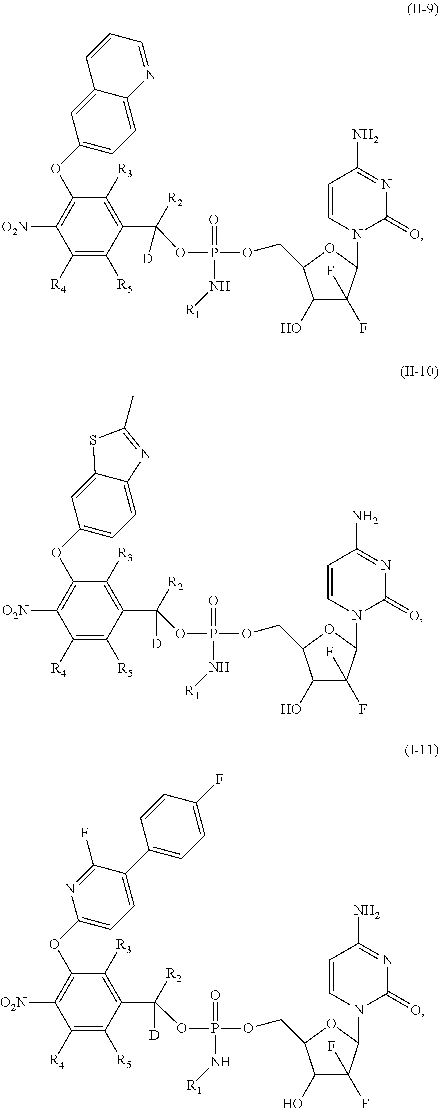

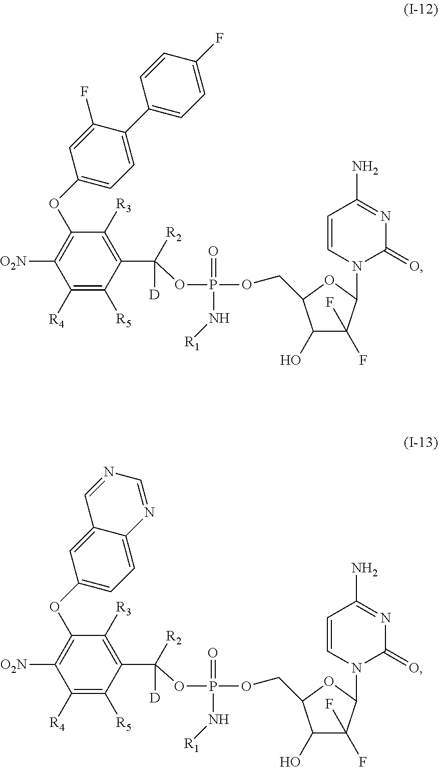

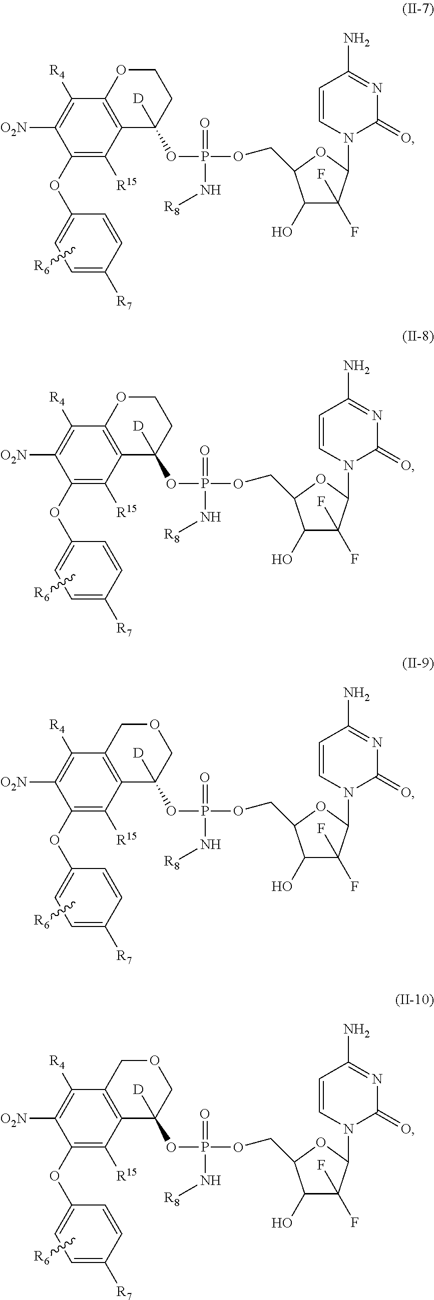

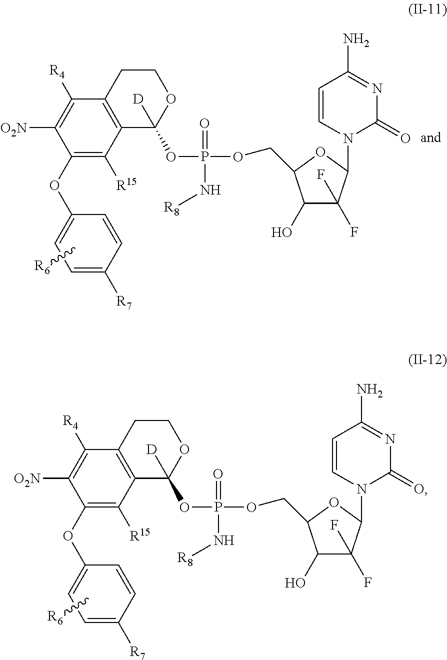

12. The compound according to claim 2 or 10 or 11, wherein, hydrogen atom(s) in R₁ are substituted by at least one deuterium.

13. The compound according to claim 2, wherein, formula (I) is selected from compounds of the following structures: 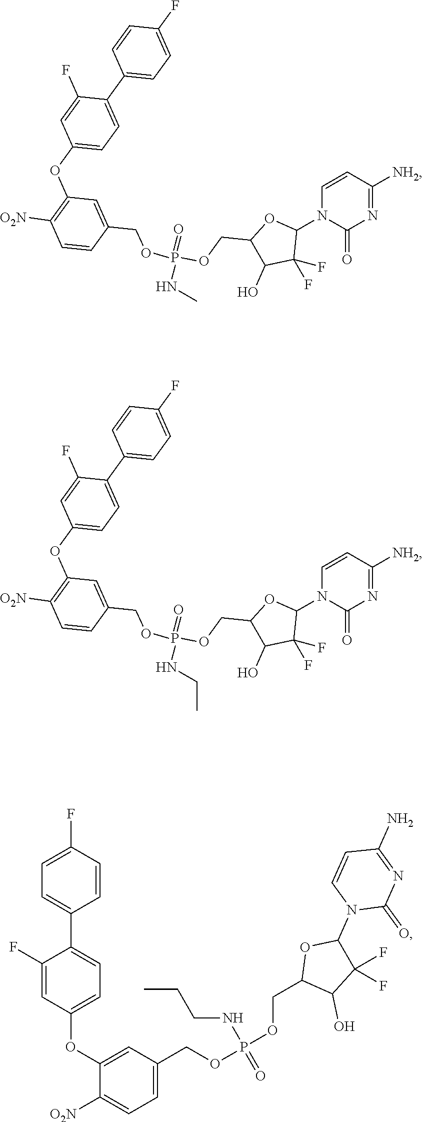 [SMILES: CNP(=O)(OCc1ccc([N+](=O)[O-])c(Oc2ccc(-c3ccc(F)cc3)c(F)c2)c1)OCC1OC(n2ccc(N)nc2=O)C(F)(F)C1O ; CCNP(=O)(OCc1ccc([N+](=O)[O-])c(Oc2ccc(-c3ccc(F)cc3)c(F)c2)c1)OCC1OC(n2ccc(N)nc2=O)C(F)(F)C1O ; CCCNP(=O)(OCc1ccc([N+](=O)[O-])c(Oc2ccc(-c3ccc(F)cc3)c(F)c2)c1)OCC1OC(n2ccc(N)nc2=O)C(F)(F)C1O] 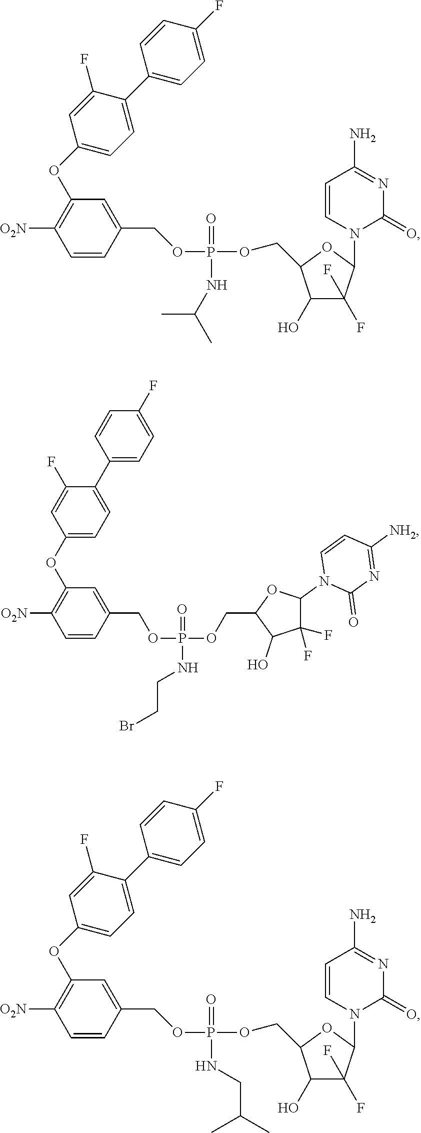 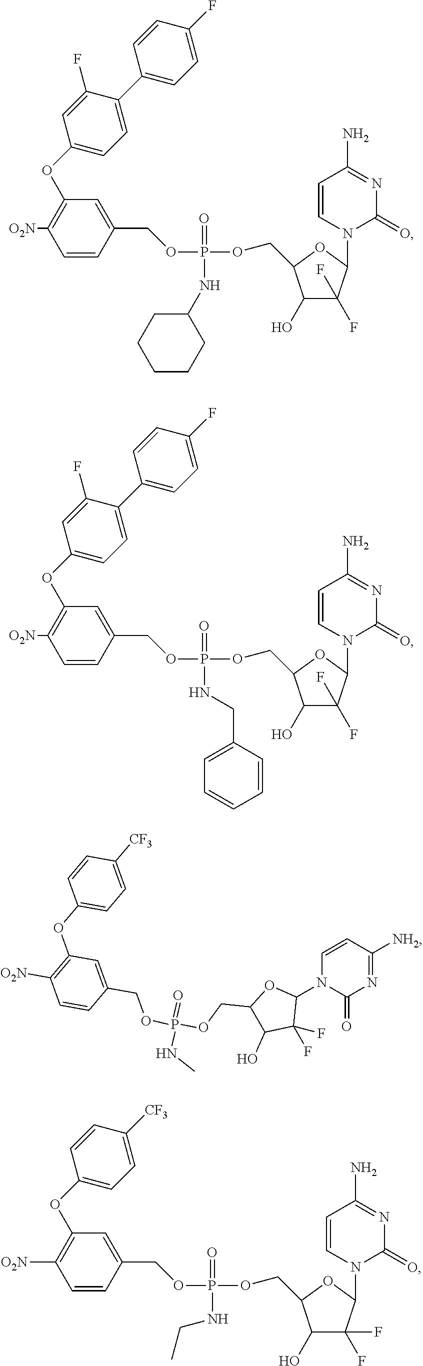 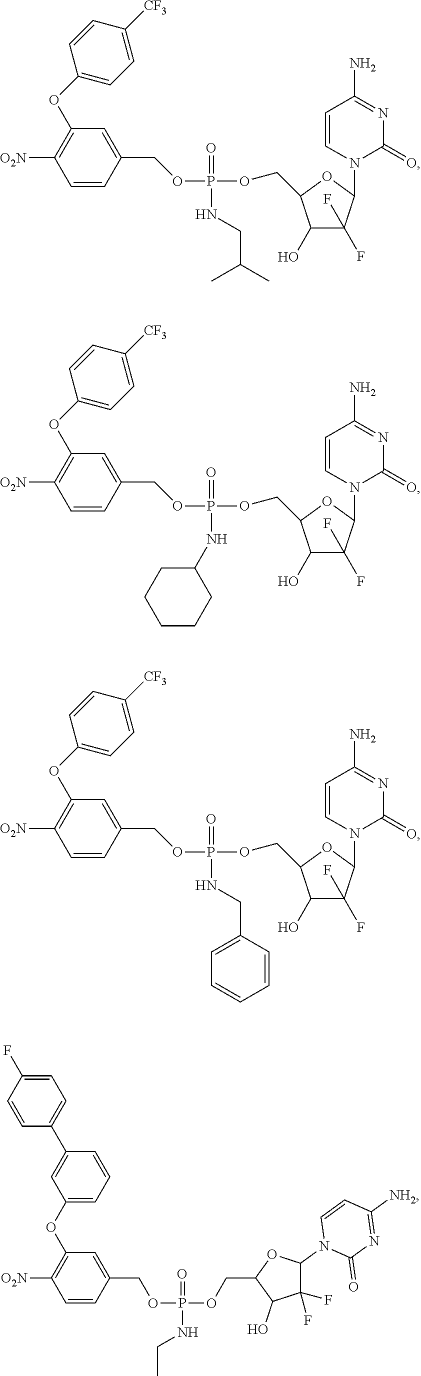 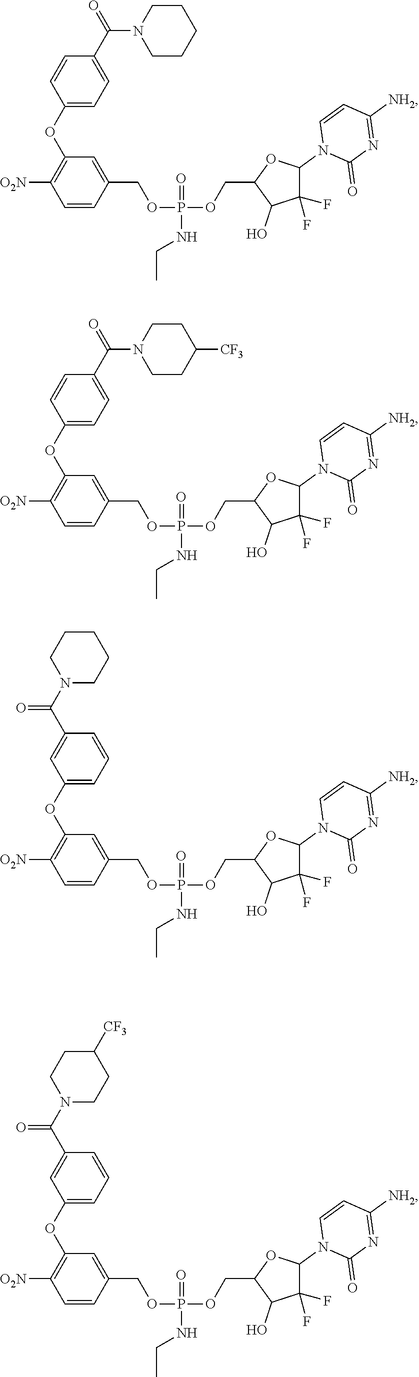 [SMILES: CCNP(=O)(OCc1ccc([N+](=O)[O-])c(Oc2ccc(C(=O)N3CCCCC3)cc2)c1)OCC1OC(n2ccc(N)nc2=O)C(F)(F)C1O ; CCNP(=O)(OCc1ccc([N+](=O)[O-])c(Oc2ccc(C(=O)N3CCC(C(F)(F)F)CC3)cc2)c1)OCC1OC(n2ccc(N)nc2=O)C(F)(F)C1O ; CCNP(=O)(OCc1ccc([N+](=O)[O-])c(Oc2cccc(C(=O)N3CCCCC3)c2)c1)OCC1OC(n2ccc(N)nc2=O)C(F)(F)C1O ; CCNP(=O)(OCc1ccc([N+](=O)[O-])c(Oc2cccc(C(=O)N3CCC(C(F)(F)F)CC3)c2)c1)OCC1OC(n2ccc(N)nc2=O)C(F)(F)C1O] 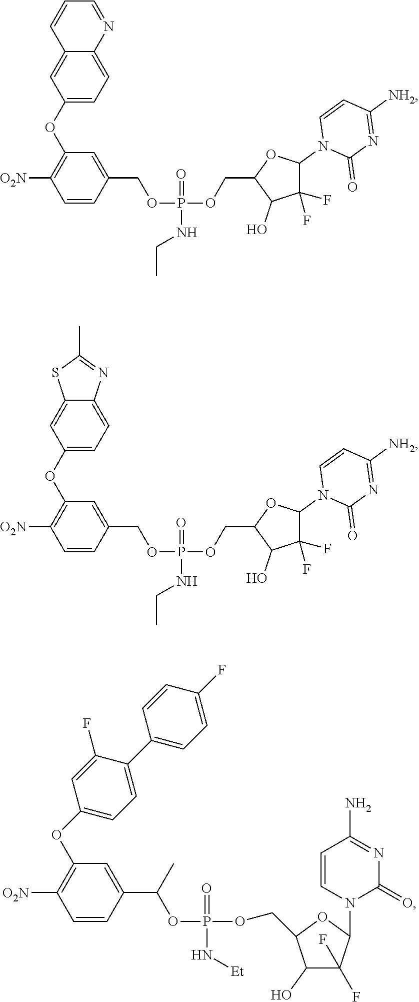 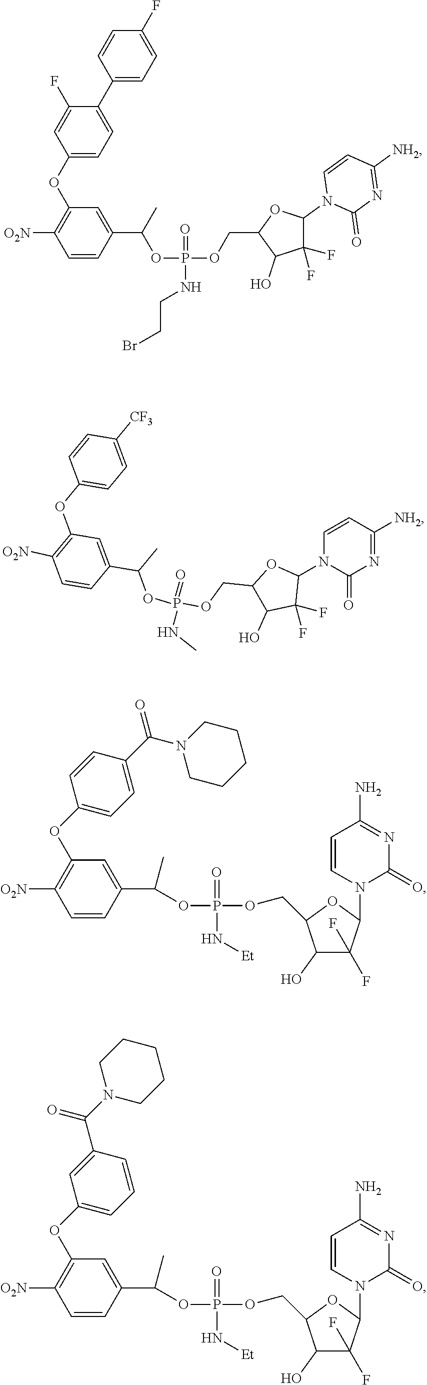 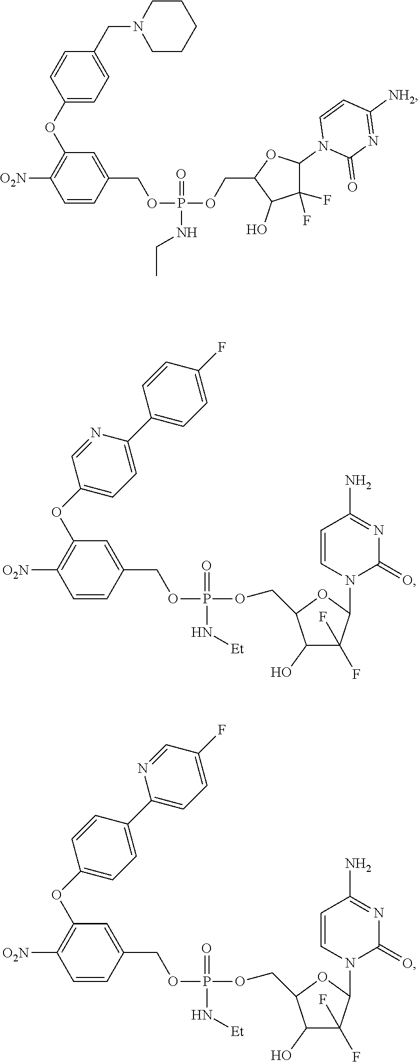 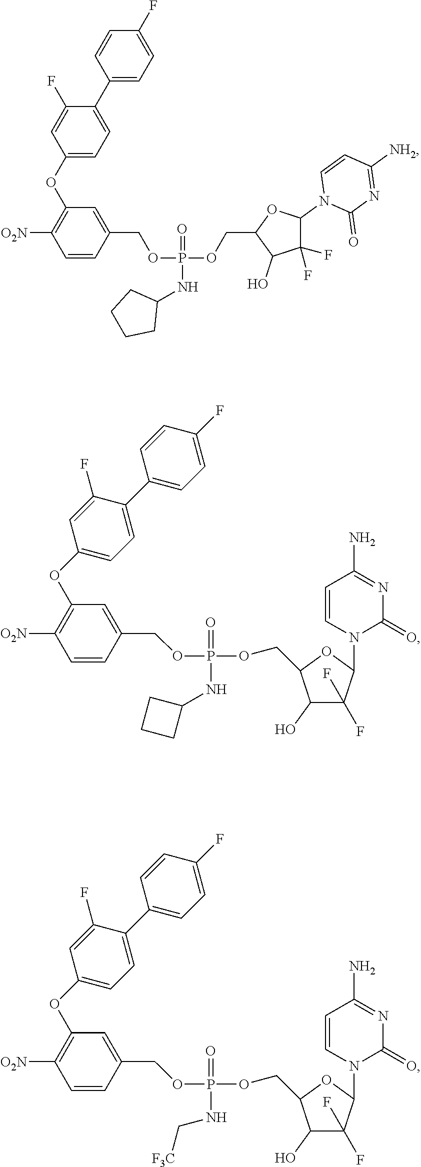 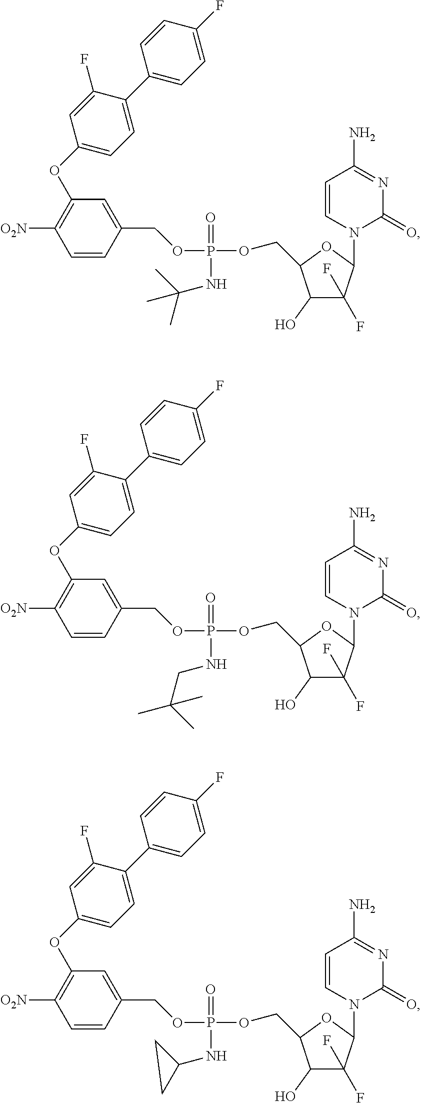 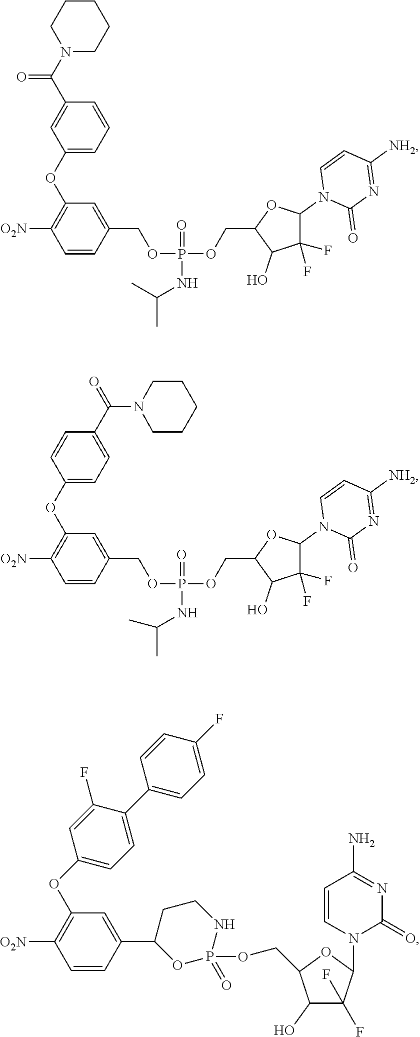 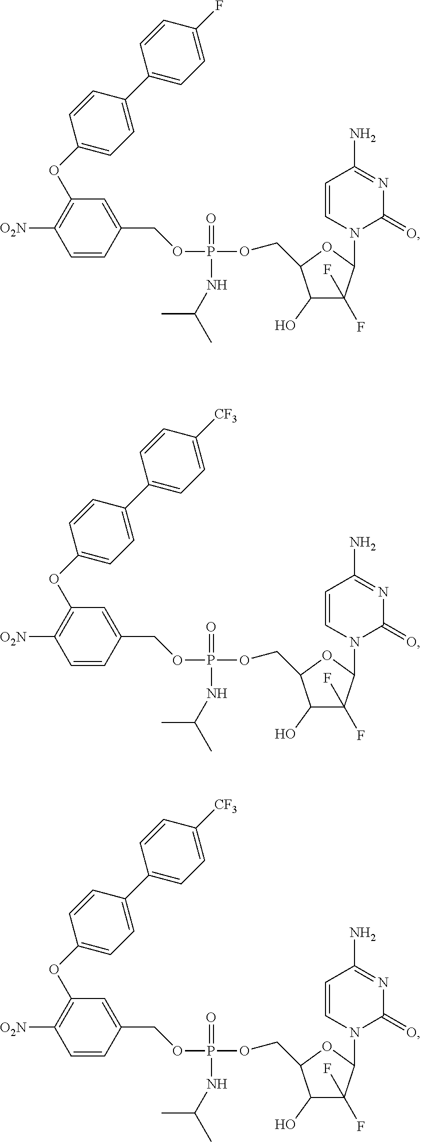 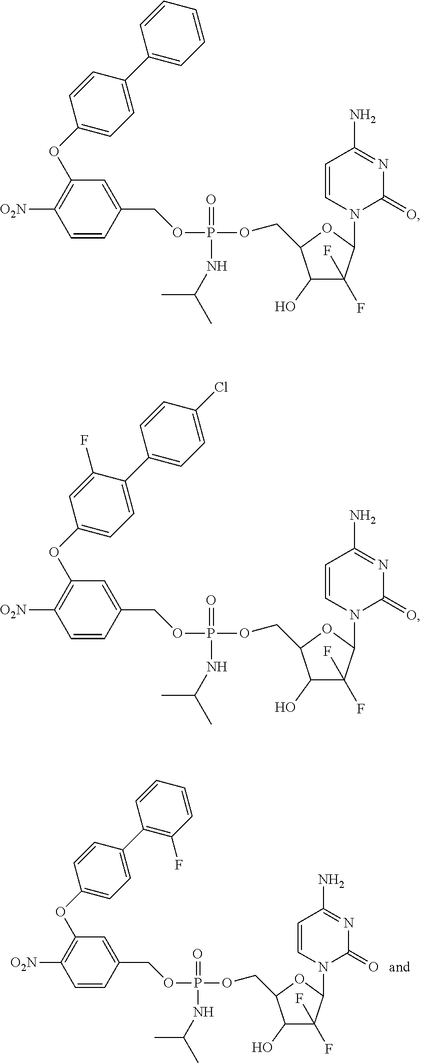 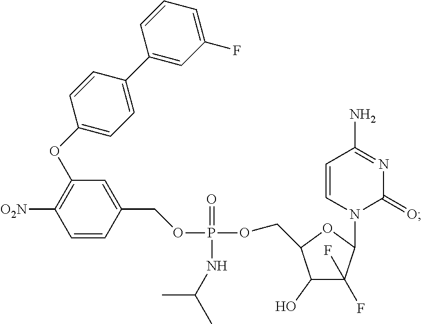
formula (II) is selected from compounds of the following structures:

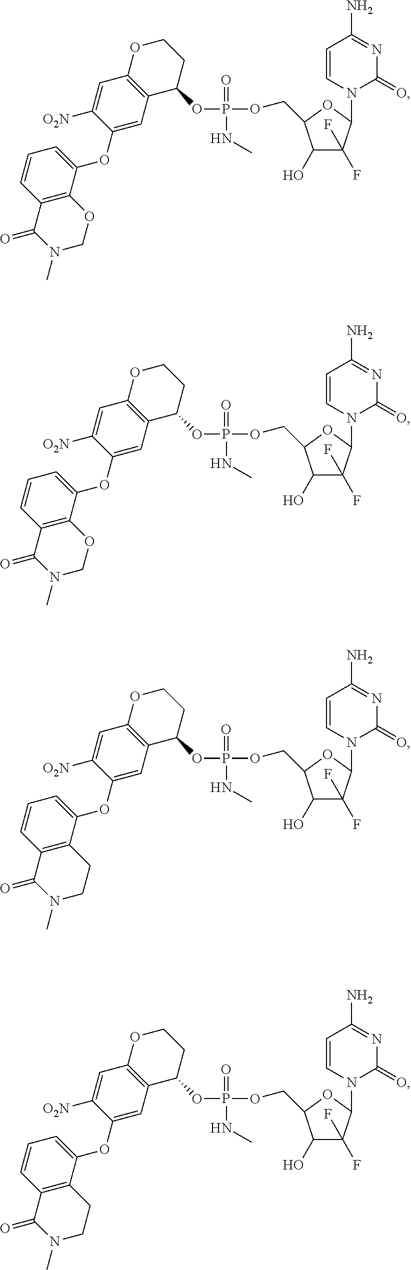

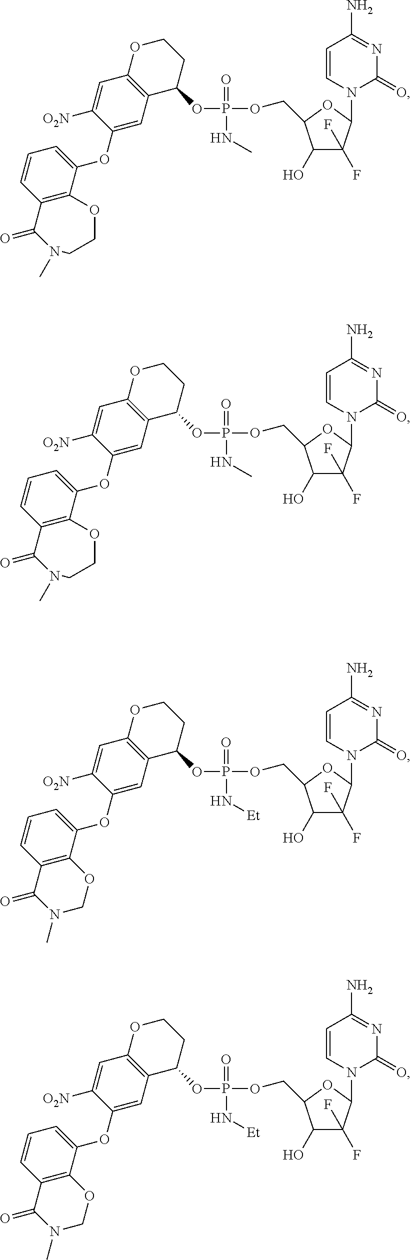

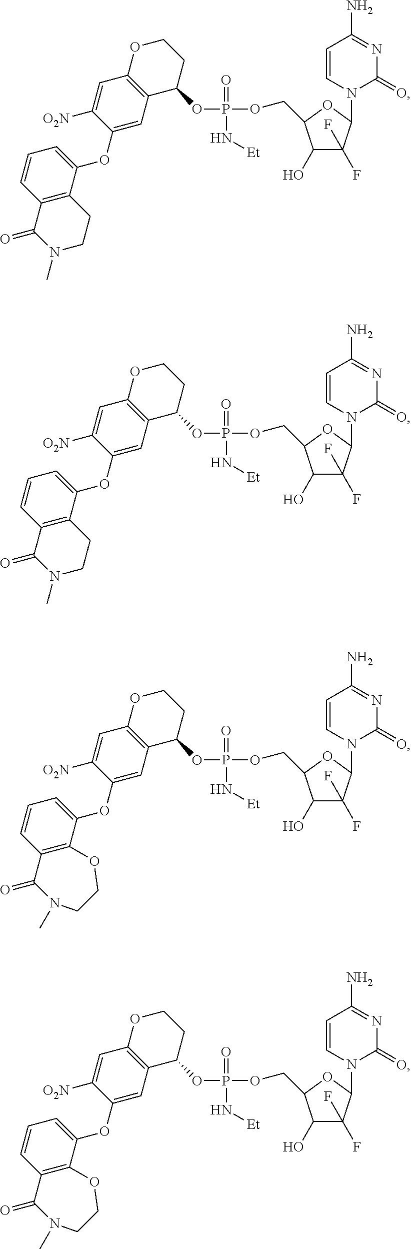

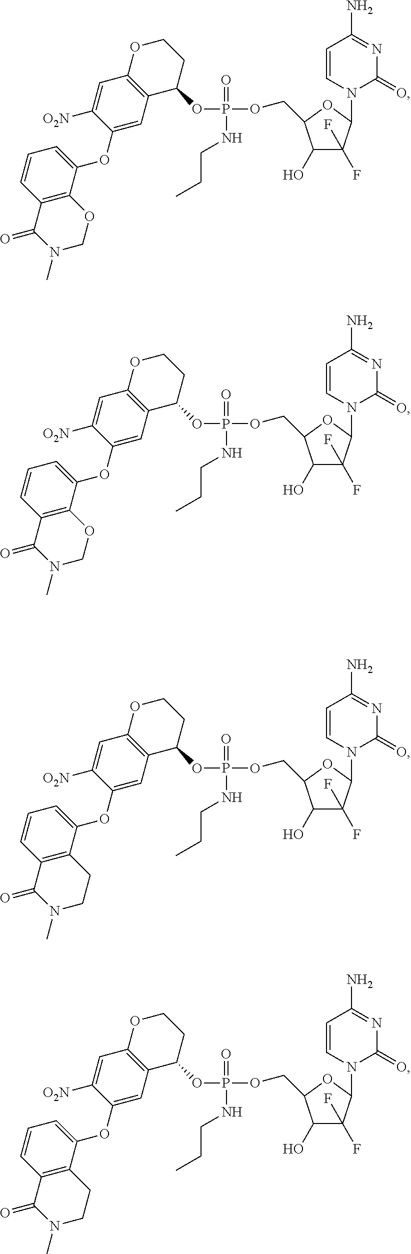

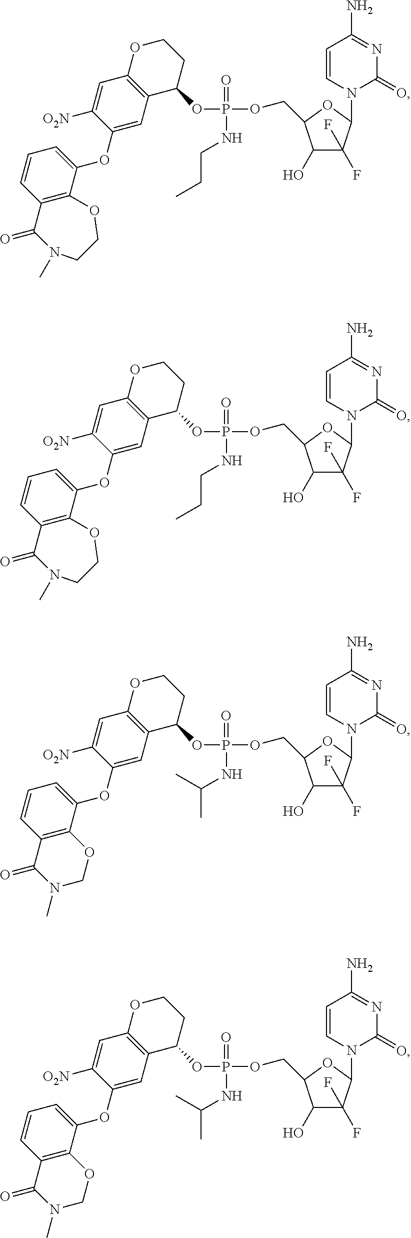

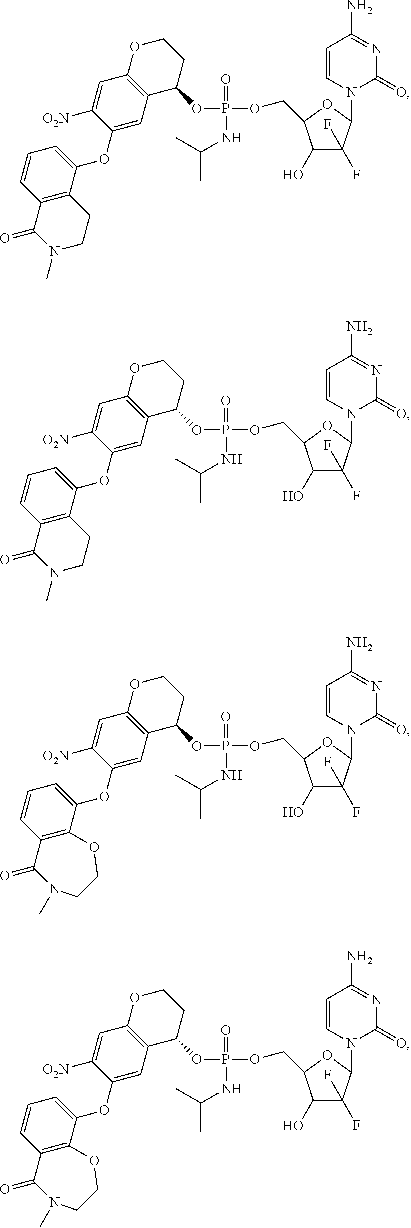

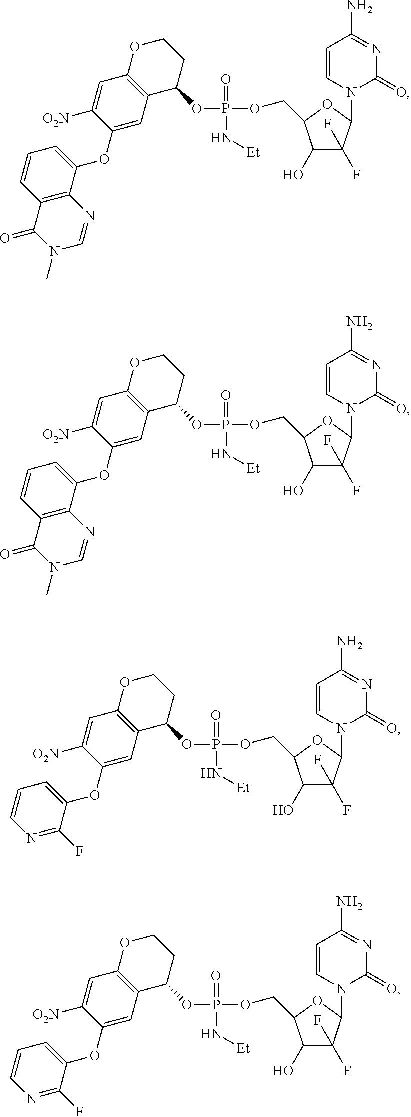

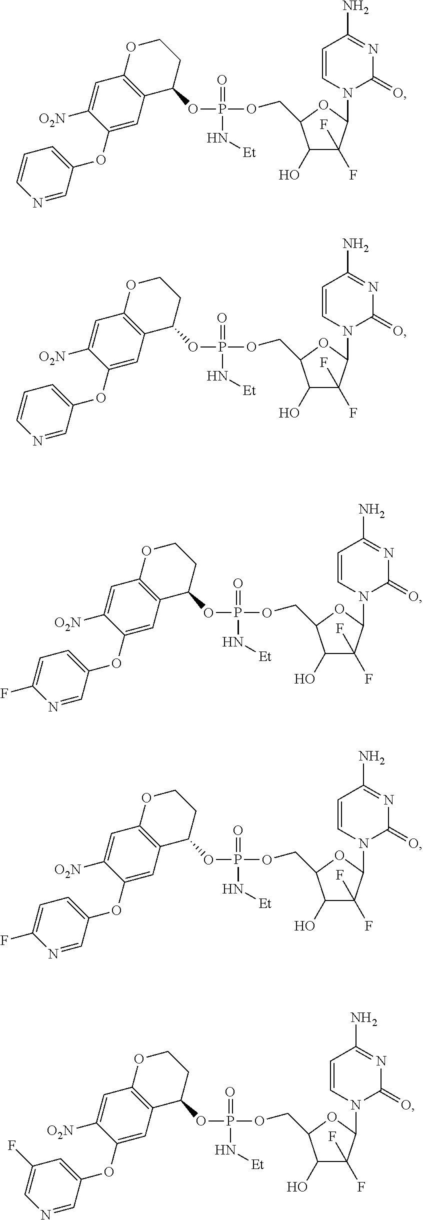

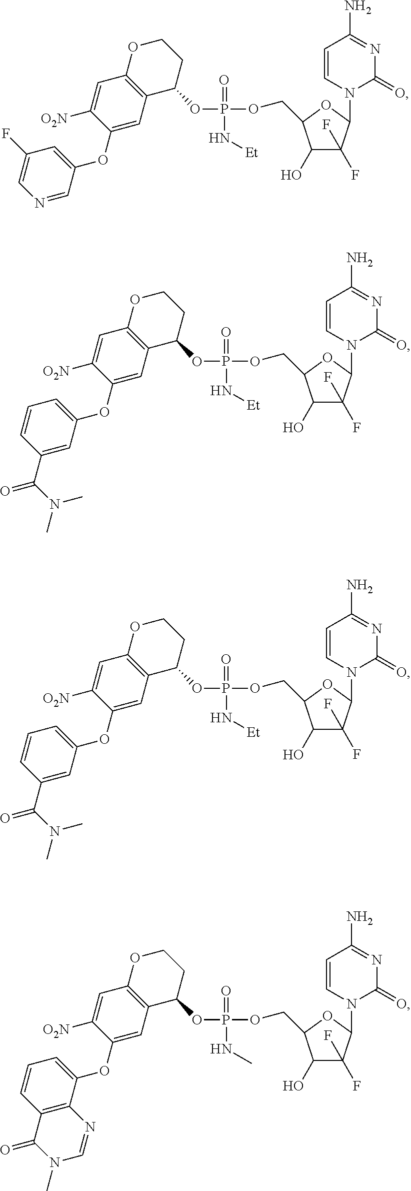

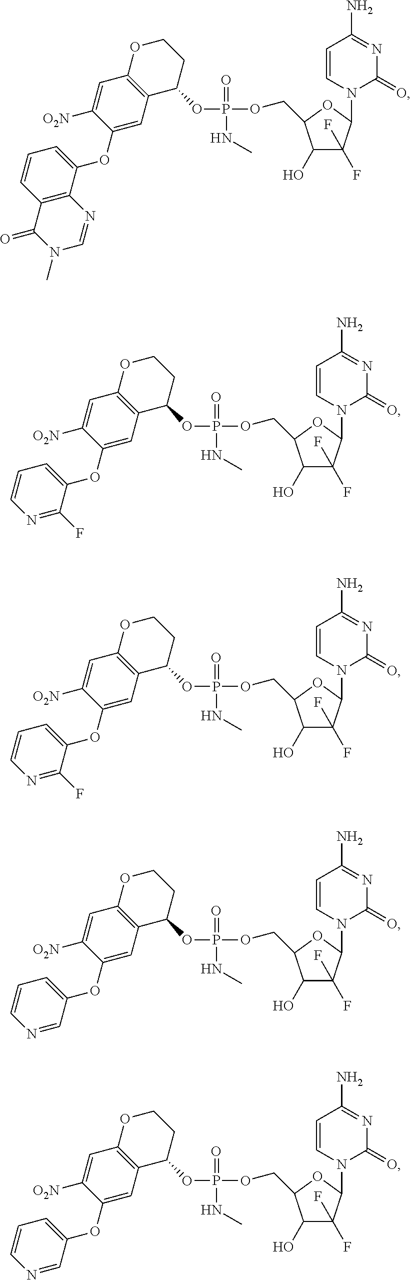

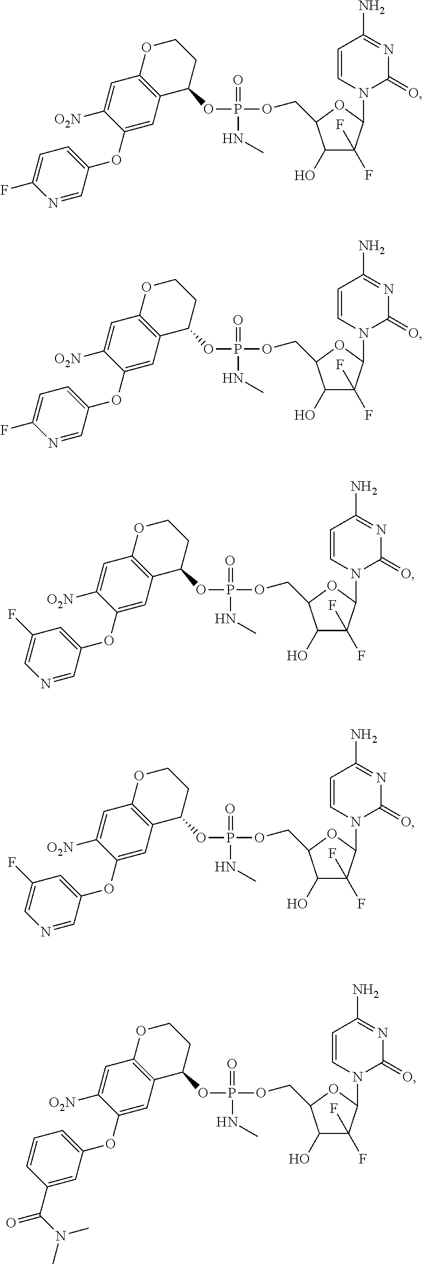

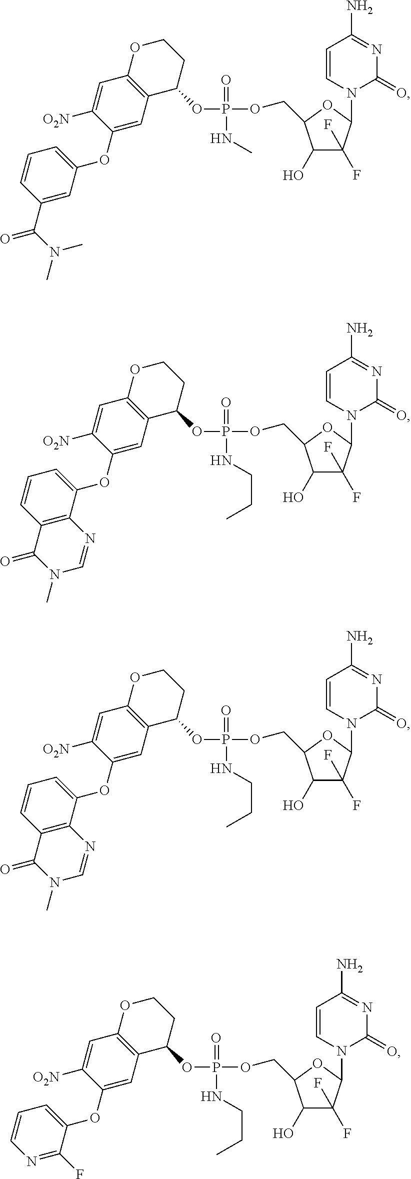

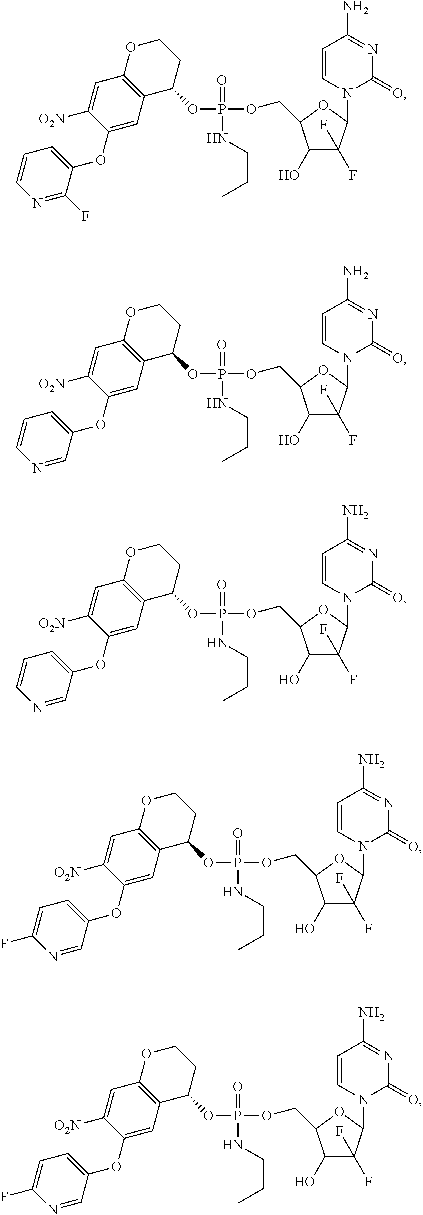

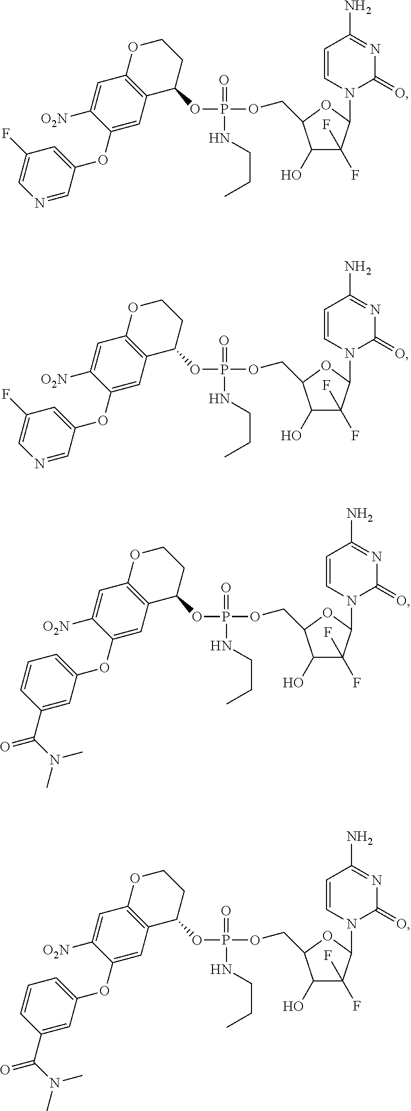

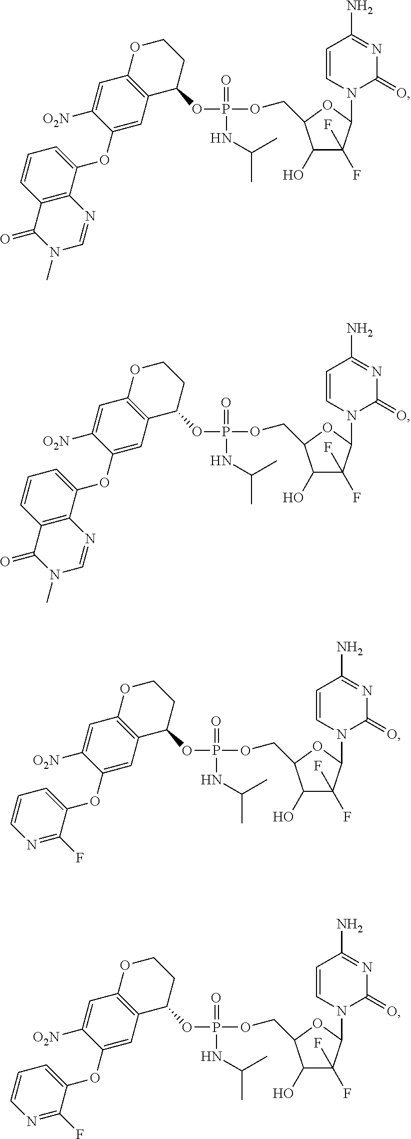

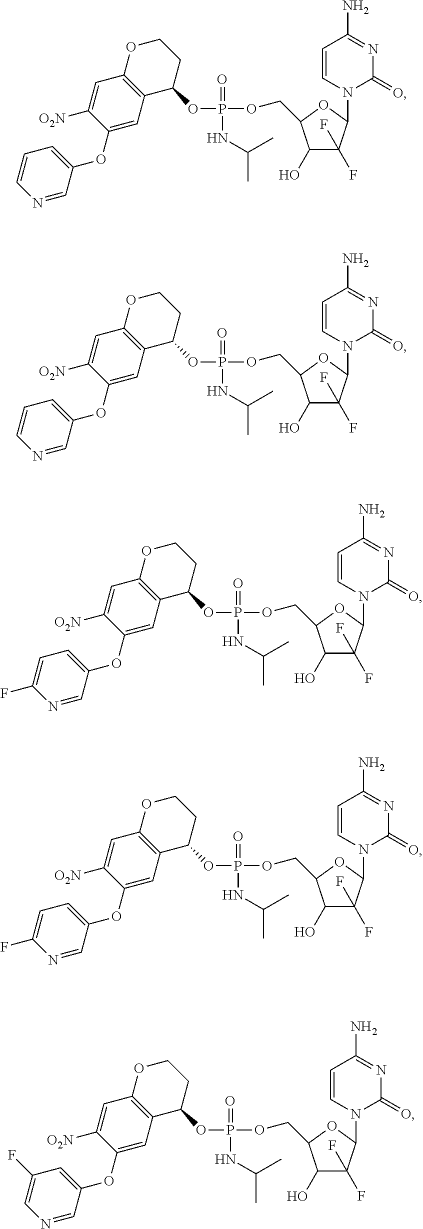

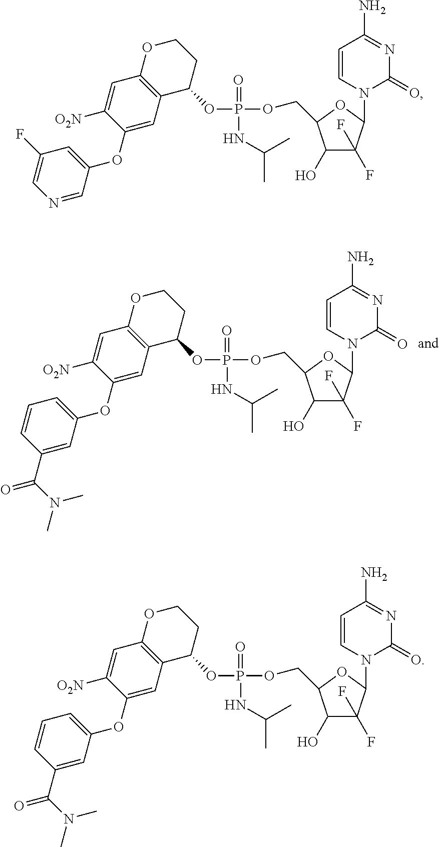

14. The compound according to any one of claims 1 to 13, wherein, the salt is a basic salt or an acid salt, and the solvate is a hydrate or alcoholate.

15. Use of the compound according to any one of claims 1 to 14 in the manufacture of a medicament for the treatment of tumors and cancers.

16. A medicament or formulation containing the compound according to any one of claims 1 to 14.

17. The medicament or formulation according to claim 16, wherein the medicament or formulation is used for the treatment of tumor and cancer diseases in patients, wherein the tumors and cancers include:
lung cancer, non-small cell lung cancer, liver cancer, pancreatic cancer, stomach cancer, bone cancer, esophagus cancer, breast cancer, prostate cancer, testicular cancer, colon cancer, ovarian cancer, bladder cancer, cervical cancer, melanoma, squamous cell carcinoma, basal cell carcinoma, adenocarcinoma, sweat gland carcinoma, sebaceous gland carcinoma, papillary carcinoma, papillary adenocarcinoma, cystic adenocarcinoma, cystic carcinoma, medullary carcinoma, bronchial carcinoma, osteocyte carcinoma, epithelial carcinoma, cholangiocarcinoma, choriocarcinoma, embryonal carcinoma, seminoma, Wilm's tumor, glioblastoma, astrocytoma, medulloblastoma, craniopharyngioma, ependymoma, pineal tumor, hemocytoblastoma, vocal cord neuroma, meningioma, neuroblastoma, optic neuroblastoma, retinoblastoma, neurofibroma, fibrosarcoma, fibroblastoma, fibroma, fibroadenoma, fibrochondroma, fibrocystoma, fibromyxoma, fibroosteoma, fibromyxosarcoma, fibropapilloma, myxosarcoma, myxocystoma, myxochondroma, myxochondrosarcoma, myxochondrofibrosarcoma, myxadenoma, myxoblastoma, liposarcoma, lipoma, lipoadenoma, lipoblastoma, lipochondroma, lipofibroma, lipoangioma, myxolipoma, chondrosarcoma, chondroma, chondromyoma, chordoma, chorioadenoma, chorioepithelioma, chorioblastoma, osteosarcoma, osteoblastoma, osteochondrofibroma, osteochondrosarcoma, osteochondroma, osteocystoma, osteodentinoma, osteofibroma, fibrosarcoma of bone, angiosarcoma, hemangioma, angiolipoma, angiochondroma, hemangioblastoma, angiokeratoma, angioglioma, angioendothelioma, angiofibroma, angiomyoma, angiolipoma, angiolymphangioma, angiolipoleiomyoma, angiomyolipoma, angiomyoneuroma, angiomyxoma, angioreticuloma, lymphangiosarcoma, lymphogranuloma, lymphangioma, lymphoma, lymphomyxoma, lymphosarcoma, lymphangiofibroma, lymphocytoma, lymphoepithelioma, lymphoblastoma, endothelioma, endothelioblastoma, synovioma, synovial sarcoma, mesothelioma, connective tissue tumor, Ewing's tumor, leiomyoma, leiomyosarcoma, leiomyoblastoma, leiomyofibroma, rhabdomyoma, rhabdomyosarcoma, rhabdomyomyxoma, acute lymphatic leukemia, acute myelogenous leukemia, anemia of chronic disease, polycythemia, lymphoma, endometrial cancer, glioma, colorectal cancer, thyroid cancer, urothelial cancer or multiple myeloma.

18. A method for treating cancer or tumor, comprising a step of applying the medicament or formulation according to claim 16; and a step of determining an AKR1C₃ reductase content or expression level of cancer cells or tissues in a patient, and
administering the medicament or formulation according to claim 16 to the patient if the measured AKR1C₃ reductase content or expression level is equal to or greater than a predetermined value.

19. A method for treating cancer or tumor, comprising a step of applying the medicament or formulation according to claim 16; and a step of adjusting an AKR1C₃ reductase content or expression level, and
administering the medicament or formulation according to claim 16 to the patient when the AKR1C₃ reductase content or expression level is adjusted to be equal to or greater than a predetermined value.
